(19) United States
(12) Patent Application Publication
(10) Pub. No.: US 2005/0245529 A1
(43) Pub. Date: Nov. 3, 2005

(54) Alkyne compounds with MCH antagonistic activity and medicaments comprising these compounds

(75) Inventors: Dirk Stenkamp, Biberach (DE); Stephan Georg Mueller, Warthausen (DE); Philipp Lustenberger, Warthausen (DE); Thorsten Lehmann-Lintz, Ochsenhausen (DE); Gerald Juergen Roth, Biberach (DE); Klaus Rudolf, Warthausen (DE); Marcus Schindler, Biberach (DE); Leo Thomas, Biberach (DE); Ralf R.H. Lotz, Schemmerhofen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/105,010

(22) Filed: Apr. 13, 2005

Related U.S. Application Data
(60) Provisional application No. 60/563,631, filed on Apr. 20, 2004.

(30) Foreign Application Priority Data
Apr. 14, 2004 (DE) 10 2004 017 930.1

(51) Int. Cl. A61K 31/501; A61K 31/444; A61K 31/4436; A61K 31/4433; C07D 49/02; C07D 41/02

(52) U.S. Cl. 514/252.01; 514/340; 514/456; 514/252.03; 544/238; 546/255; 546/283.4

(57) ABSTRACT

Alkyne compounds of formula I 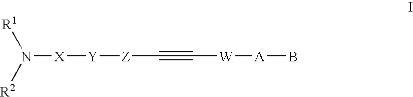
wherein A, B, W, X, Y, Z, R¹, and R² have the meanings given herein, which have MCH-receptor antagonistic activity and are useful for preparing pharmaceutical compositions for the treatment of metabolic disorders and/or eating disorders, particularly obesity and diabetes.

### RELATED APPLICATIONS

This application claims benefit of U.S. Ser. No. 60/563,63 1, filed Apr. 20, 2004, and claims priority to German Application No. 10 2004 017 930.1, filed Apr. 14, 2004, each of which is hereby incorporated by reference in its entirety.

### FIELD OF THE INVENTION

The present invention relates to new alkyne compounds, the physiologically acceptable salts thereof as well as their use as MCH antagonists and their use in preparing a pharmaceutical preparation which is suitable for the prevention and/or treatment of symptoms and/or diseases caused by MCH or causally connected with MCH in some other way. The invention also relates to the use of a compound according to the invention for influencing eating behavior and for reducing body weight and/or for preventing any increase in body weight in a mammal. It further relates to compositions and medicaments containing a compound according to the invention and processes for preparing them. Other aspects of this invention relate to processes for preparing the compounds according to the invention.

### BACKGROUND OF THE INVENTION

The intake of food and its conversion in the body is an essential part of life for all living creatures. Therefore, deviations in the intake and conversion of food generally lead to problems and also illness. The changes in the lifestyle and nutrition of humans, particularly in industrialized countries, have promoted morbid overweight (also known as corpulence or obesity) in recent decades. In affected people, obesity leads directly to restricted mobility and a reduction in the quality of life. There is the additional factor that obesity often leads to other diseases such as, for example, diabetes, dyslipidemia, high blood pressure, arteriosclerosis, and coronary heart disease. Moreover, high bodyweight alone puts an increased strain on the support and mobility apparatus, which can lead to chronic pain and diseases such as arthritis or osteoarthritis. Thus, obesity is a serious health problem for society.

The term obesity means an excess of adipose tissue in the body. In this connection, obesity is fundamentally to be seen as the increased level of fatness which leads to a health risk. There is no sharp distinction between normal individuals and those suffering from obesity, but the health risk accompanying obesity is presumed to rise continuously as the level of fatness increases. For simplicity's sake, in the present invention, individuals with a Body Mass Index (BMI), which is defined as the bodyweight measured in kilograms divided by the height (in meters) squared, above a value of 25 and more particularly above 30, are preferably regarded as suffering from obesity.

Apart from physical activity and a change in nutrition, there is currently no convincing treatment option for effectively reducing bodyweight. As obesity is a major risk factor in the development of serious and even life-threatening diseases, however, it is all the more important to have access to pharmaceutical active substances for the prevention and/or treatment of obesity. One approach which has been proposed very recently is the therapeutic use of MCH antagonists (cf. inter alia WO 01/21577 and WO 01/82925).

Melanin-concentrating hormone (MCH) is a cyclic neuropeptide consisting of 19 amino acids. It is synthesized predominantly in the hypothalamus in mammals and from there travels to other parts of the brain by the projections of hypothalamic neurons. Its biological activity is mediated in humans through two different glycoprotein-coupled receptors (GPCRs) from the family of rhodopsin-related GPCRs, namely the MCH receptors 1 and 2 (MCH-1R, MCH-2R).

Investigations into the function of MCH in animal models have provided good indications for a role of the peptide in regulating the energy balance, i.e., changing metabolic activity and food intake. D. Qu, et al., A role for melanin-concentrating hormone in the central regulation of feeding behavior, Nature, 1996, 380(6571): pp. 243-7; M. Shimada, et al., Mice lacking melanin-concentrating hormone are hypophagic and lean, Nature, 1998, 396(6712): pp. 670-4. For example, after intraventricular administration of MCH in rats, food intake was increased compared with control animals. Additionally, transgenic rats which produce more MCH than control animals, when given a high-fat diet, responded by gaining significantly more weight than animals without an experimentally altered MCH level. It was also found that there is a positive correlation between phases of increased desire for food and the quantity of MCH MRNA in the hypothalamus of rats. However, experiments with MCH knock-out mice are particularly important in showing the function of MCH. Loss of the neuropeptide results in lean animals with a reduced fat mass, which take in significantly less food than control animals.

The anorectic effects of MCH are presumably mediated in rodents through the G-Galpha i-coupled MCH-1R [B. Borowsky, et al., Antidepressant, anxiolytic and anorectic effects of a melanin-concentrating hormone-1 receptor antagonist, Nat Med, 2002, 8(8): pp. 825-30; Y. Chen, et al., Targeted disruption of the melanin-concentrating hormone receptor-1 results in hyperphagia and resistance to diet-induced obesity, Endocrinology, 2002, 143(7): pp. 2469-77; D. J. Marsh, et al., Melanin-concentrating hormone 1 receptor-deficient mice are lean, hyperactive, and hyperphagic and have altered metabolism. Proc Natl Acad Sci USA, 2002, 99(5): pp. 3240-5; S. Takekawa, et al., T-226296: A novel, orally active and selective melanin-concentrating hormone receptor antagonist, Eur J Pharmacol, 2002, 438(3): pp. 129-35.], as, unlike primates, ferrets, and dogs, no second MCH receptor subtype has hitherto been found in rodents. After losing the MCH-1R, knock-out mice have a lower fat mass, an increased energy conversion and, when fed on a high fat diet, do not put on weight, compared with control animals. Another indication of the importance of the MCH system in regulating the energy balance results from experiments with a receptor antagonist (SNAP-7941). B. Borowsky, et al., Nat Med, 2002, 8(8): pp. 825-30. In long term trials, the animals treated with the antagonist lose significant amounts of weight.

In addition to its anorectic effect, the MCH-1R antagonist SNAP-7941 also achieves additional anxiolytic and antidepressant effects in behavioral experiments on rats. B. Borowsky, et al., Nat Med, 2002, 8(8): pp. 825-30. Thus, there are clear indications that the MCH-MCH-1R system is involved not only in regulating the energy balance but also in affectivity.

In the patent literature certain amine compounds are proposed as MCH antagonists. Thus, WO 01/21577 (Takeda) describes compounds of formula 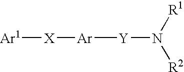
wherein Ar¹ denotes a cyclic group, X denotes a spacer, Y denotes a bond or a spacer, Ar denotes an aromatic ring which may be fused with a non-aromatic ring, R¹ and R² independently of one another denote H or a hydrocarbon group, while R¹ and R² together with the adjacent N atom may form an N-containing hetero ring and R² with Ar may also form a spirocyclic ring, and R together with the adjacent N atom and Y may form an N-containing hetero ring, as MCH antagonists for the treatment of obesity.

Moreover WO 01/82925 (Takeda) also describes compounds of formula 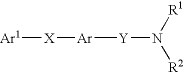
wherein Ar¹ denotes a cyclic group, X and Y represent spacer groups, Ar denotes an optionally substituted fused polycyclic aromatic ring, R¹ and R² independently of one another represent H or a hydrocarbon group, while R¹ and R² together with the adjacent N atom may form an N-containing heterocyclic ring and R² together with the adjacent N atom and Y may form an N-containing hetero ring, as MCH antagonists for the treatment of obesity, inter alia.

WO 2004/024702 proposes carboxylic acid amide compounds of formula I 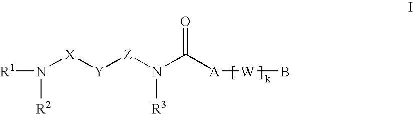
wherein Y, A, and B may represent cyclic groups and X, Z, and W may denote bridges or bonds, as MCH-antagonists.

WO 04/039780 A1 describes alkyne compounds of formula I 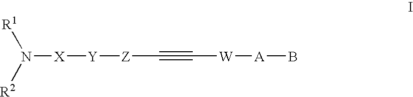
wherein Y, A, and B may denote cyclic groups and X, Z, and W may denote bridges or bonds, as MCH-antagonists. The following substances are mentioned, inter alia: (1-{5-[5-(4-chlorophenyl)pyridin-2-ylethynyl]pyridin-2-yl}pyrrolidin-3-yl)dimethylamine, 5′-[5-(4-chlorophenyl)pyridin-2-ylethynyl]-3-pyrrolidin-1-yl-3,4,5,6-tetrahydro-2H-[1,2′]-bipyridinyl, 1′-{5-[5-(4-chlorophenyl)pyridin-2-ylethynyl]pyridin-2-yl}-[1,3′]-bipyrrolidinyl, {5-[5-(4-chlorophenyl)pyridin-2-ylethynyl]pyridin-2-yl}-(2-pyrrolidin-1-ylpropyl)amine, 5-(4-chlorophenyl)-2-[4-(1-methyl-2-piperidin-1-ylethoxy)phenylethynyl]pyridine, 5-(4-chlorophenyl)-2-[4-(3-piperidin-1-ylpyrrolidin-1-yl)phenylethynyl]pyridine, 5-(4-chlorophenyl)-2-{4-[2-(4-methylpiperidin-1-yl)propoxy]phenylethynyl}pyridine, (1-{5-[5-(4-chlorophenyl)pyridin-2-ylethynyl]pyridin-2-yl}pyrrolidin-3-yl)-4-methylpiperidine, 5-(4-chlorophenyl)-2-[4-(2-methyl-2-piperidin-1-ylpropoxy)phenylethynyl]pyridine, 5-(4-chlorophenyl)-2-{4-[3-(4-methylpiperidin-1-yl)cyclohexyl]phenylethynyl}pyridine, 5-(4-chlorophenyl)-2-{4-[3-(4-methylpiperidin-1-yl)cyclohex-1-enyl]phenylethynyl}pyridine, 5-(4-chlorophenyl)-2-{4-[3-(4-methylpiperidin-1-yl)cyclopent-1-enyl]phenylethynyl}pyridine, 5-(4-chlorophenyl)-2-{4-[3-(4-methylpiperidin-1-yl)cyclopentyl]phenylethynyl}pyridine, 5-(4-chlorophenyl)-2-[4-(3-pyrrolidin-1-ylpropenyl)phenylethynyl]pyridine, and 5-(4-chlorophenyl)-2-[4-(3-pyrrolidin-1-ylprop-1-ynyl)phenylethynyl]pyridine.

WO 04/039764 A1 describes amide compounds of formula I 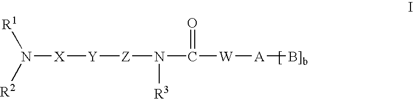
wherein Y, A, and B may denote cyclic groups and X denotes an alkylene bridge, Z denotes a bridge or bond and W is selected from the group comprising —CR^{6a}R^{6b}—O, —CR^{7a}═CR^{7c}, —CR^{6a}R^{6b}—NR⁸, —CR^{7a}R^{7b}—CR^{7c}R^{7d}—, and —NR⁸—CR^{6a}R^{6b}, as MCH-antagonists.

The aim of the present invention is to identify new alkyne compounds, particularly those which are especially effective as MCH antagonists. The invention also sets out to provide new alkyne compounds which can be used to influence the eating habits of mammals and achieve a reduction in body weight, particularly in mammals, and/or prevent an increase in body weight.

The present invention further sets out to provide new pharmaceutical compositions which are suitable for the prevention and/or treatment of symptoms and/or diseases caused by MCH or otherwise causally connected to MCH. In particular, the aim of this invention is to provide pharmaceutical compositions for the treatment of metabolic disorders such as obesity and/or diabetes as well as diseases and/or disorders which are associated with obesity and diabetes. Other objectives of the present invention are concerned with demonstrating advantageous uses of the compounds according to the invention. The invention also sets out to provide a process for preparing the amide compounds according to the invention. Other aims of the present invention will be immediately apparent to the skilled person from the foregoing remarks and those that follow.

In a first aspect, the present invention relates to alkyne compounds of general formula I 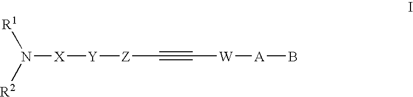
wherein:
R¹, R² independently of one another denote H, C₁₋₈-alkyl, C₃₋₇-cycloalkyl, or a phenyl or pyridinyl group optionally mono- or polysubstituted by identical or different groups R²⁰ and/or monosubstituted by nitro, while the alkyl or cycloalkyl group may be mono- or polysubstituted by identical or different groups R¹¹, and a —CH₂— group in position 3 or 4 of a 5-, 6-, or 7-membered cycloalkyl group may be replaced by —O—, —S—, or —NR³—, or
R¹ and R² form a C₃₋₈alkylene bridge, wherein a —CH₂— group not adjacent to the N atom of the R¹R²N— group may be replaced by —CH═N—, —CH═CH—, —O—, —S—, —SO—, —(SO₂)—, —CO—, —C(═CH₂)—, or —NR¹³—, while in the alkylene bridge defined hereinbefore one or more H atoms may be replaced by identical or different groups R¹⁴, and the alkylene bridge defined hereinbefore may be substituted by one or two identical or different carbo- or heterocyclic groups Cy such that the bond between the alkylene bridge and the group Cy is made via a single or double bond, via a common C atom forming a spirocyclic ring system, via two common adjacent C and/or N atoms forming a fused bicyclic ring system, or via three or more C and/or N atoms forming a bridged ring system;
X denotes a C₁₋₆-alkylene bridge which comprises one or more substituents selected independently of one another from fluorine, chlorine, hydroxy, cyano, CF₃, C₁₋₄-alkyl, hydroxy-C₁₋₄-alkyl, C₃₋₆-cycloalkyl, and C₁₋₄-alkoxy, while two alkyl substituents may be joined together forming a C₃₋₇-cycloalkyl group, or
   a C₂₋₄-alkylenoxy or C₂₋₄-alkyleneimino bridge, while the imino group may be substituted by a C₁₋₄-alkyl group, and wherein the alkylene unit comprises one or more substituents selected independently of one another from fluorine, CF₃, hydroxy-C₁₋₄-alkyl, C¹⁻⁴-alkyl, and C₃₋₆-cycloalkyl, while two alkyl groups may be joined together forming a C₃₋₇-cycloalkyl group or if an alkyl group is linked to the imino group, may also be joined together to form a cyclo-C₄₋₆-alkyleneimino group, or
   a C₃₋₆-alkenylene or C₃₋₆-alkynylene bridge which is unsubstituted or comprises one or more substituents selected independently of one another from fluorine, chlorine, CF₃, hydroxy-C¹⁻⁴-alkyl, C¹⁻⁴-alkyl, and C₃₋₆-cycloalkyl, while two alkyl substituents may be joined together forming a C₃₋₇-cycloalkyl or C₅₋₇-cycloalkenyl group;
W and Z independently of one another denote a single bond or a C₁₋₂-alkylene bridge, while two adjacent C atoms may be joined together with an additional C₁₋₄-alkylene bridge, and one or two C atoms independently of one another may be substituted by one or two identical or different C₁₋₃-alkyl groups, while two alkyl groups may be joined together to form a carbocyclic ring;
Y and A independently of one another are selected from the group of the bivalent cyclic groups phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, chromanyl, chromen-4-onyl, thienyl, furanyl, benzothienyl, or benzofuranyl, while the abovementioned cyclic groups may be mono- or polysubstituted at one or more C atoms by identical or different groups R²⁰, in the case of a phenyl ring may also additionally be monosubstituted by nitro, and/or one or more NH groups may be substituted by R²¹;
B has one of the meanings given for Y or A or denotes C₁₋₆-alkyl, C₁₋₆-alkenyl, C₁₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, or C₃₋₇-cycloalkyl-C₁₋₃-alkynyl, wherein one or more C atoms independently of one another may be mono- or polysubstituted by halogen and/or may be monosubstituted by hydroxy or cyano and/or cyclic groups may be mono- or polysubstituted by identical or different groups R²⁰;
Cy denotes a carbo- or heterocyclic group selected from one of the following meanings a saturated 3- to 7-membered carbocyclic group, an unsaturated 4- to 7-membered carbocyclic group, a phenyl group, a saturated 4- to 7-membered or unsaturated 5- to 7-membered heterocyclic group with an N, O, or S atom as heteroatom, a saturated or unsaturated 5- to 7-membered heterocyclic group with two or more N atoms or with one or two N atoms and an O or S atom as heteroatoms, or an aromatic heterocyclic 5- or 6-membered group with one or more identical or different heteroatoms selected from N, O, and/or S, while the abovementioned saturated 6- or 7-membered groups may also be present as bridged ring systems with an imino, (C₁₋₄-alkyl)-imino, methylene, (C¹⁻⁴-alkyl)-methylene, or di-(C₁₋₄-alkyl)-methylene bridge, and
   the abovementioned cyclic groups may be mono- or polysubstituted at one or more C atoms by identical or different groups R²⁰, in the case of a phenyl group may also additionally be monosubstituted by nitro, and/or one or more NH groups may be substituted by R²¹;
R¹¹ denotes halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N—, R¹⁸R¹⁹N—CO—, or Cy, while in the abovementioned groups one or more C atoms may be substituted independently of one another by substituents selected from halogen, OH, CN, CF₃, C₁₋₃-alkyl, and hydroxy-C₁₋₃-alkyl;
R¹³ has one of the meanings given for R¹⁷;
R¹⁴ denotes halogen, cyano, C₁₋₆-alkyl, C₂₋₆-alkenyl, C₂₋₆-alkynyl, R¹⁵—O—, R¹⁵—O—CO—, R¹⁵—CO—, R¹⁵—CO—O—, R¹⁶R¹⁷N—, R¹⁸R¹⁹N—CO—, R¹⁵—O—C₁₋₃-alkyl, R¹⁵—O—CO—C₁₋₃-alkyl, R¹⁵—SO₂—NH—, R¹⁵—O—CO—NH—C₁₋₃-alkyl, R¹⁵—SO₂—NH—C₁₋₃-alkyl, R¹⁵—CO—C₁₋₃-alkyl, R¹⁵—CO—O—C₁₋₃-alkyl, R¹⁶R¹⁷N—C₁₋₃-alkyl, R¹⁸R¹⁹N—CO—C₁₋₃-alkyl, or Cy-C₁₋₃-alkyl;
R¹⁵ denotes H, C₁₋₄-alkyl, C₃₋₇-cycloalkyl, C₃₋₇-cycloalkyl-C₁₋₃-alkyl, phenyl, phenyl-C¹⁻³-alkyl, pyridinyl, or pyridinyl-C₁₋₃-alkyl;
R¹⁶ denotes H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C¹⁻⁴-alkyl-amino-C₂₋₆-alkyl, di-(C₁₋₄-alkyl)-amino-C₂₋₆-alkyl, or cyclo-C₃₋₆-alkyleneimino-C₂₋₆-alkyl;
R¹⁷ has one of the meanings given for R¹⁶ or denotes phenyl, phenyl-C₁₋₃-alkyl, pyridinyl, C¹⁻⁴-alkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N-(C₁₋₄-alkylcarbonyl)-N-(C₁₋₄-alkyl)-amino-C₂₋₃-alkyl, C₁₋₄-alkylsulfonyl, C₁₋₄-alkylsulfonylamino-C₂₋₃-alkyl, or N-(C₁₋₄-alkylsulfonyl)-N(-C₁₋₄-alkyl)-amino-C₂₋₃-alkyl;
R¹⁸ and R¹⁹ independently of one another denote H or C₁₋₆-alkyl;
R²⁰ denotes halogen, hydroxy, cyano, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, R²²-C₁₋₃-alkyl, or has one of the meanings given for R²²;
R²¹ denotes C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulfonyl, aminosulfonyl, C₁₋₄-alkylaminosulfonyl, di-C₁₋₄-alkylaminosulfonyl, or cyclo-C₃₋₆-alkylene-iminosulfonyl;
R²² denotes pyridinyl, phenyl, phenyl-C₁₋₃-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-aminocarbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonyl-amino, cyclo-C₃₋₆-alkyleneimino, phenyl-C₁₋₃-alkylamino, N-(C₁₋₄-alkyl)phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino, or C₁₋₄-alkylaminocarbonylamino,
   while in the abovementioned groups and radicals, particularly in W, Z, and R¹³ to R²², in each case one or more C atoms may additionally be mono- or polysubstituted by F and/or in each case one or two C atoms independently of one another are additionally monosubstituted by Cl or Br and/or in each case one or more phenyl rings independently of one another may additionally comprise one, two or three substituents selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl, and di-(C₁₋₃-alkyl)-amino-C₁₋₃-alkyl, and/or may be monosubstituted by nitro, and

the H atom of any carboxy group present or an H atom bound to an N atom may in each case be replaced by a group which can be cleaved in vivo,

the tautomers, the diastereomers, the enantiomers, the mixtures thereof, and the salts thereof,

while the following compounds are not included in the invention:
(1-{5-[5-(4-chlorophenyl)pyridin-2-ylethynyl]pyridin-2-yl}pyrrolidin-3-yl)dimethylamine,
5′-[5-(4-chlorophenyl)pyridin-2-ylethynyl]-3-pyrrolidin-1-yl-3,4,5,6-tetrahydro-2H-[1,2′]-bipyridinyl,
1′-{5-[5-(4-chlorophenyl)pyridin-2-ylethynyl]pyridin-2-yl}-[1,3′]-bipyrrolidinyl,
{5-[5-(4-chlorophenyl)pyridin-2-ylethynyl]pyridin-2-yl}-(2-pyrrolidin-1-ylpropyl)amine,
5-(4-chlorophenyl)-2-[4-(1-methyl-2-piperidin-1-ylethoxy)phenylethynyl]pyridine,
5-(4-chlorophenyl)-2-[4-(3-piperidin-1-ylpyrrolidin-1-yl)phenylethynyl]pyridine,
5-(4-chlorophenyl)-2-{4-[2-(4-methylpiperidin-1-yl)propoxy]phenylethynyl}pyridine,
(1-{5-[5-(4-chlorophenyl)pyridin-2-ylethynyl]pyridin-2-yl}pyrrolidin-3-yl)-4-methylpiperidine,
5-(4-chlorophenyl)-2-[4-(2-methyl-2-piperidin-1-ylpropoxy)phenylethynyl]pyridine,
5-(4-chlorophenyl)-2-{4-[3-(4-methylpiperidin-1-yl)cyclohexyl]phenylethynyl}pyridine,
5-(4-chlorophenyl)-2-{4-[3-(4-methylpiperidin-1-yl)cyclohex-1-enyl]phenylethynyl}pyridine,
5-(4-chlorophenyl)-2-{4-[3-(4-methylpiperidin-1-yl)cyclopent-1-enyl]phenylethynyl}pyridine,
5-(4-chlorophenyl)-2-{4-[3-(4-methylpiperidin-1-yl)cyclopentyl]phenylethynyl}pyridine,
5-(4-chlorophenyl)-2-[4-(3-pyrrolidin-1-ylpropenyl)phenylethynyl]pyridine,
5-(4-chlorophenyl)-2-[4-(3-pyrrolidin-1-ylprop-1-ynyl)phenylethynyl]pyridine.

The compounds according to the present invention, including the physiologically acceptable salts, are especially effective, compared with known, structurally similar compounds, as antagonists of the MCH receptor, particularly the MCH-1 receptor, and exhibit very good affinity in MCH receptor binding studies. In addition, the compounds according to the invention have a high to very high selectivity with regard to the MCH receptor. Generally the compounds according to the invention have low toxicity, they are well absorbed by oral route and have good intracerebral transitivity, particularly brain accessibility.

The invention also relates to the compounds in the form of the individual optical isomers, mixtures of the individual enantiomers or racemates, in the form of the tautomers and in the form of the free bases or corresponding acid addition salts with pharmacologically acceptable acids.

The subject of the invention also includes the compounds according to the invention, including their salts, wherein one or more hydrogen atoms are replaced by deuterium.

This invention also includes the physiologically acceptable salts of the alkyne compounds according to the invention as described above and hereinafter.

Also covered by this invention are compositions containing at least one alkyne compound according to the invention and/or a salt according to the invention optionally together with one or more physiologically acceptable excipients.

Also covered by this invention are pharmaceutical compositions containing at least one alkyne compound according to the invention and/or a salt according to the invention optionally together with one or more inert carriers and/or diluents.

This invention also relates to the use of at least one alkyne compound according to the invention and/or a salt according to the invention for influencing the eating behavior of a mammal.

The invention further relates to the use of at least one alkyne compound according to the invention and/or a salt according to the invention for reducing the body weight and/or for preventing an increase in the body weight of a mammal.

The invention also relates to the use of at least one alkyne compound according to the invention and/or a salt according to the invention for preparing a pharmaceutical composition with an MCH receptor-antagonistic activity, particularly with an MCH-1 receptor-antagonistic activity.

This invention also relates to the use of at least one alkyne compound according to the invention and/or a salt according to the invention for preparing a pharmaceutical composition which is suitable for the prevention and/or treatment of symptoms and/or diseases which are caused by MCH or are otherwise causally connected with MCH.

A further object of this invention is the use of at least one alkyne compound according to the invention and/or a salt according to the invention for preparing a pharmaceutical composition which is suitable for the prevention and/or treatment of metabolic disorders and/or eating disorders, particularly obesity, bulimia, bulimia nervosa, cachexia, anorexia, anorexia nervosa, and hyperphagia.

The invention also relates to the use of at least one alkyne compound according to the invention and/or a salt according to the invention for preparing a pharmaceutical composition which is suitable for the prevention and/or treatment of diseases and/or disorders associated with obesity, particularly diabetes, especially type II diabetes, complications of diabetes including diabetic retinopathy, diabetic neuropathy, diabetic nephropathy, insulin resistance, pathological glucose tolerance, encephalorrhagia, cardiac insufficiency, cardiovascular diseases, particularly arteriosclerosis and high blood pressure, arthritis, and gonitis.

In addition the present invention relates to the use of at least one alkyne compound according to the invention and/or a salt according to the invention for preparing a pharmaceutical composition which is suitable for the prevention and/or treatment of hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, and hormonal disorders.

The invention also relates to the use of at least one alkyne compound according to the invention and/or a salt according to the invention for preparing a pharmaceutical composition which is suitable for the prevention and/or treatment of urinary problems, such as for example urinary incontinence, overactive bladder, urgency, nycturia, and enuresis.

The invention further relates to the use of at least one alkyne compound according to the invention and/or a salt according to the invention for preparing a pharmaceutical composition which is suitable for the prevention and/or treatment of dependencies and/or withdrawal symptoms.

The invention further relates to processes for preparing for preparing a pharmaceutical composition according to the invention, characterized in that at least one alkyne compound according to the invention and/or a salt according to the invention is incorporated in one or more inert carriers and/or diluents by a non-chemical method.

The invention also relates to a pharmaceutical composition containing a first active substance which is selected from the alkyne compounds according to the invention and/or the corresponding salts as well as a second active substance which is selected from the group consisting of active substances for the treatment of diabetes, active substances for the treatment of diabetic complications, active substances for the treatment of obesity, preferably other than MCH antagonists, active substances for the treatment of high blood pressure, active substances for the treatment of dyslipidemia or hyperlipidemia, including arteriosclerosis, active substances for the treatment of arthritis, active substances for the treatment of anxiety states, and active substances for the treatment of depression, optionally together with one or more inert carriers and/or diluents.

Moreover, in one aspect, the invention relates to a process for preparing alkyne compounds of formula A.5
R¹R²N—X—Y—C≡C—W-A-B (A.5)
while in formulae A.1, A.2, A.3, A.4, and A.5, R¹, R², X, Y, W, A, and B have one of the meanings given hereinbefore and hereinafter,

wherein a halogen compound of formula A.1
HO—X—Y-Hal (A.1)
wherein Hal denotes chlorine, bromine, or iodine, preferably bromine or iodine, is reacted with an alkyne compound of formula A.2
H—C≡C—W-A-B (A.2)
in the presence of a suitable palladium catalyst, a suitable base, and copper (I) iodide in a suitable solvent, and the compound of formula A.3 obtained
HO—X—Y—C≡C—W-A-B (A.3)
is reacted with methanesulfonic acid chloride (MsCl) to produce the methanesulfonate derivative A.4,
MsO-X—Y—C≡C—W-A-B (A.4)
which is further reacted with an amine of formula H—NR¹R² to form the end product A.5.

This invention further relates to a process for preparing alkyne compounds of formula B.5
R¹R²N—X—Y-Z-C≡C-A-B (B.5)
while in formulae B.1, B.2, B.3, B.4, and B.5, R¹, R², X, Y, Z, A, and B have one of the meanings given hereinbefore and hereinafter, wherein a halogen compound of formula B. 1
Hal-A-B (B. I)
wherein Hal denotes chlorine, bromine, or iodine, preferably bromine or iodine, is reacted with an alkyne compound of formula B.2
HO—X—Y-Z-C≡C—H (B.2)
in the presence of a suitable palladium catalyst, a suitable base, and copper (I) iodide in a suitable solvent, and the resulting compound of formula B.3
HO—X—Y-Z-C≡C-A-B (B.3)
is reacted with methanesulfonic acid chloride (MsCl) to form the methanesulfonate derivative B.4,
MsO-X—Y-Z-C≡C-A-B (B.4)
which is reacted further with an amine of formula H—NR¹R² to form the end product B.5.

In addition, the invention relates to a process for preparing alkyne compounds of formula C.3
R¹R²N—X—Y—C≡C—W-A-B (C.3)
while in formulae C.1, C.2, and C.3, R¹, R², X, Y, W, A, and B have one of the meanings given hereinbefore and hereinafter, wherein a halogen compound of formula C. 1
R¹R²N—X—Y-Hal (C. 1)
wherein Hal denotes chlorine, bromine, or iodine, preferably bromine or iodine, is further reacted with an alkyne compound of formula C.2
H—C≡C—W-A-B (C.2)
in the presence of a suitable palladium catalyst, a suitable base and copper (I) iodide in a suitable solvent to yield the end product C.3.

In another aspect, the invention relates to a process for preparing alkyne compounds of formula D.3
R¹R²N—X—Y-Z-C≡C-A-B (D.3)
while in formulae D.1, D.2, and D.3, R¹, R², X, Y, Z, A, and B have one of the meanings given hereinbefore and hereinafter,

wherein a halogen compound of formula D.2
Hal-A-B (D.2)
wherein Hal denotes chlorine, bromine, or iodine, preferably bromine or iodine, is reacted with an alkyne compound of formula D. 1
R¹R²N—X—Y-Z-C≡C—H (D. 1)
in the presence of a suitable palladium catalyst, a suitable base, and copper (I) iodide in a suitable solvent to form the end product D.3.

The starting materials and intermediate products used in the synthesis according to the invention are also a subject of this invention.

### DETAILED DESCRIPTION OF THE INVENTION

Unless otherwise specified, the groups, residues, and substituents, particularly A, B, W, X, Y, Z, Cy, R¹, R², R¹¹, and R¹³ to R²², have the meanings given hereinbefore.

If groups, residues and/or substituents occur more than once in a compound, they may have the same or different meanings in each case.

If R¹ and R² are not joined together via an alkylene bridge, R¹ and R² independently of one another preferably denote a C₁₋₈-alkyl or C₃₋₇-cycloalkyl group mono- or polysubstituted by identical or different groups R¹¹, while a —CH₂— group in position 3 or 4 of a 5-, 6-, or 7-membered cycloalkyl group may be replaced by —O—, —S—, or —NR¹³—, or a phenyl or pyridinyl group optionally mono- or polysubstituted by identical or different groups R²⁰ and/or monosubstituted by nitro, while one or both of the groups R¹ and R² may also represent H.

Preferred meanings of the group R¹¹ are F, Cl, Br, C₁₋₆-alkyl, C₂₋₆-alkenyl, C₂₋₆-alkynyl, R¹⁵—O—, cyano, R¹⁶R¹⁷N—, C₃₋₇-cycloalkyl, cyclo-C₃₋₆-alkyleneimino, pyrrolidinyl, N-(C₁₋₄-alkyl)pyrrolidinyl, piperidinyl, N-(C₁₋₄-alkyl)piperidinyl, phenyl, and pyridyl, while in the abovementioned groups and radicals one or more C atoms may be mono- or polysubstituted independently of one another by F, C₁₋₃-alkyl or hydroxy-C₁₋₃-alkyl, and/or one or two C atoms may be monosubstituted independently of one another by Cl, Br, OH, CF₃, or CN, and the abovementioned cyclic groups may be mono- or polysubstituted at one or more C atoms by identical or different radicals R²⁰, or in the case of a phenyl group may also additionally be monosubstituted by nitro, and/or one or more NH groups may be substituted by R²¹. If R¹¹ has one of the meanings R¹⁵—O—, cyano, R¹⁶R¹⁷N or cyclo-C₃₋₆-alkyleneimino, the C atom of the alkyl or cycloalkyl group substituted by R¹¹ is preferably not directly connected to a heteroatom, such as for example the group —N—X.

Preferably the groups R¹, R² independently of one another represent H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC—C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N—(C₁₋₄-alkyl)pyrrolidin-3-yl, pyrrolidinyl-C₁₋₃-alkyl, N—(C₁₋₄-alkyl)pyrrolidinyl-C₁₋₃-alkyl, piperidin-3-yl, piperidin-4-yl, N—(C₁₋₄-alkyl)piperidin-3-yl, N—(C₁₋₄-alkyl)piperidin-4-yl, piperidinyl-C₁₋₃-alkyl, N—(C₁₋₄-alkyl)piperidinyl-C₁₋₃-alkyl, tetrahydropyran-3-yl, tetrahydropyran-4-yl, phenyl, phenyl-C₁₋₃-alkyl, pyridyl or pyridyl-C₁₋₃-alkyl, while in the abovementioned groups and radicals one or more C atoms independently of one another may be mono- or polysubstituted by F, C₁₋₃-alkyl or hydroxy-C₁₋₃-alkyl, and/or one or two C atoms independently of one another may be monosubstituted by Cl, Br, OH, CF₃, or CN, and the abovementioned cyclic groups may be mono- or polysubstituted at one or more C atoms by identical or different radicals R²⁰, in the case of a phenyl group may also additionally be monosubstituted by nitro, and/or one or more NH groups may be substituted by R²¹. Preferred substituents of the abovementioned phenyl or pyridyl groups are selected from the group F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylamino, aminocarbonyl, difluoromethoxy, trifluoromethoxy, amino-C₁₋₃-alkyl, C₁₋₃-alkylamino-C₁₋₃-alkyl, and di-(C₁₋₃-alkyl)-amino-C₁₋₃-alkyl, while a phenyl group may also be monosubstituted by nitro.

Particularly preferred definitions of the groups R¹ and/or R² are selected from the group consisting of H, C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, (hydroxy-C₃₋₇-cycloalkyl)-C₁₋₃-alkyl, ω-(C₁₋₄-alkoxy)-C₂₋₃-alkyl, pyridyl and benzyl, while an alkyl, cycloalkyl, or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl, or CN.

Most particularly preferred groups R¹ and/or R² are selected from the group consisting of H, methyl, ethyl, n-propyl, isopropyl, prop-2-enyl, but-2-enyl, prop-2-ynyl, but-2-ynyl, 2-methoxyethyl, cyclopropyl, cyclopentyl, cyclohexyl, cyclopropylmethyl, cyclopentylmethyl, hydroxy-C₃₋₇-cycloalkyl, (hydroxy-C₁₋₃-alkyl)-hydroxy-C₃₋₇-cycloalkyl, dihydroxy-C₃₋₅-alkyl, (1-hydroxy-C₃₋₆-cycloalkyl)-methyl, tetrahydropyran-3-yl, tetrahydropyran-4-yl, 2-hydroxyethyl, 3-hydroxypropyl, benzyl, and pyridyl, while the abovementioned groups may be mono- or polysubstituted by F and/or C₁₋₃-alkyl, and the phenyl and pyridyl rings may be substituted as specified.

Examples of most particularly preferred groups R¹ and/or R² are therefore H, methyl, ethyl, n-propyl, isopropyl, prop-2-enyl, prop-2-ynyl, 2-methoxyethyl, cyclopropyl, cyclopentyl, cyclohexyl, cyclopropylmethyl, cyclopentylmethyl, hydroxycyclopentyl, hydroxycyclohexyl, (hydroxymethyl)hydroxycyclopentyl, (hydroxymethyl)hydroxycyclohexyl, 2,3-dihydroxypropyl, 2-hydroxy-1-(hydroxymethyl)ethyl, 1,1-di(hydroxymethyl)ethyl, (1-hydroxycyclopropyl)methyl, tetrahydropyran-3-yl, tetrahydropyran-4-yl, 2-hydroxyethyl, 3-hydroxypropyl, benzyl, and pyridyl.

Particularly preferably, at least one of the groups R¹ and R², and most particularly preferably both groups, have a meaning other than H.

If R¹ and R² form an alkylene bridge, this is preferably a C₃₋₇-alkylene bridge or a C₃₋₇-alkylene bridge, particularly a C₃₋₆-alkylene bridge, wherein a —CH₂— group not adjacent to the N atom of the R¹R²N— group is replaced by —CH═N—, —CH═CH—, —O—, —S—, —CO—, or —NR¹³—, while in the alkylene bridge defined hereinbefore one or more H atoms may be replaced by identical or different groups R¹⁴, and the alkylene bridge defined hereinbefore may be substituted with a carbo- or heterocyclic group Cy in such a way that the bond between the alkylene bridge and the group Cy is made via a single or double bond, via a common C atom forming a spirocyclic ring system, via two common adjacent C- and/or N atoms forming a fused bicyclic ring system, or via three or more C- and/or N atoms forming a bridged ring system.

Preferably also, R¹ and R² form an alkylene bridge such that R¹R²N— denotes a group which is selected from azetidine, pyrrolidine, piperidine, azepan, 2,5-dihydro-1H-pyrrole, 1,2,3,6-tetrahydropyridine, 2,3,4,7-tetrahydro-1H-azepine, 2,3,6,7-tetrahydro-1H-azepine, piperazine in which the free imine function is substituted by R¹³, piperidin-4-one, morpholine, and thiomorpholine, is particularly preferably selected from pyrrolidine, piperidine, piperazine in which the free imine function is substituted by R¹³, and morpholine, while according to the general definition of R¹ and R², one or more H atoms may be replaced by identical or different groups R¹⁴, and/or the abovementioned groups may be substituted by one or two identical or different carbo- or heterocyclic groups Cy in a manner specified according to the general definition of R¹ and R², while the group Cy may be mono- or polysubstituted by R²⁰.

Particularly preferred groups Cy are C₃₋₇-cycloalkyl, aza-C₄₋₇-cycloalkyl, particularly cyclo-C₃₋₆-alkyleneimino, as well as 1-C₁₋₄-alkyl-aza-C₄₋₇-cycloalkyl, while the group Cy may be mono- or polysubstituted by R²⁰.

The C₃₋₈-alkylene bridge formed by R¹ and R², wherein —CH₂— groups may be replaced as specified, may be substituted, as described, by one or two identical or different carbo- or heterocyclic groups Cy, which may be substituted as specified hereinbefore.

In the event that the alkylene bridge is linked to a group Cy through a single bond, Cy is preferably selected from the group consisting of C₃₋₇-cycloalkyl, cyclo-C₃₋₆-alkyleneimino, 1H-imidazole, thienyl, and phenyl.

In the event that the alkylene bridge is linked to a group Cy via a common C atom forming a spirocyclic ring system, Cy is preferably selected from the group consisting of C₃₋₇-cycloalkyl, aza-C₄₋₈-cycloalkyl, oxa-C₄₋₈-cycloalkyl, and 2,3-dihydro-1H-quinazolin-4-one.

In the event that the alkylene bridge is linked to a group Cy via two common adjacent C and/or N atoms forming a fused bicyclic ring system, Cy is preferably selected from the group consisting of C₄₋₇-cycloalkyl, phenyl, and thienyl.

In the event that the alkylene bridge is linked to a group Cy via three or more C and/or N atoms forming a bridged ring system, Cy preferably denotes C₄₋₈-cycloalkyl or aza-C₄₋₈-cycloalkyl.

In the event that the heterocyclic group R¹R²N— is substituted by a group Cy, the group Cy is preferably linked to the group R¹R²N— through a single bond, while Cy is preferably selected from the group consisting of C₃₋₇-cycloalkyl and cyclo-C₃₋₆-alkyleneimino, while these groups may be substituted as specified, preferably by fluorine, CF₃, C₁₋₃-alkyl, hydroxy-C₁₋₃-alkyl, and hydroxy.

Particularly preferably, the group 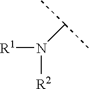
is therefore defined according to one of the following partial formulae 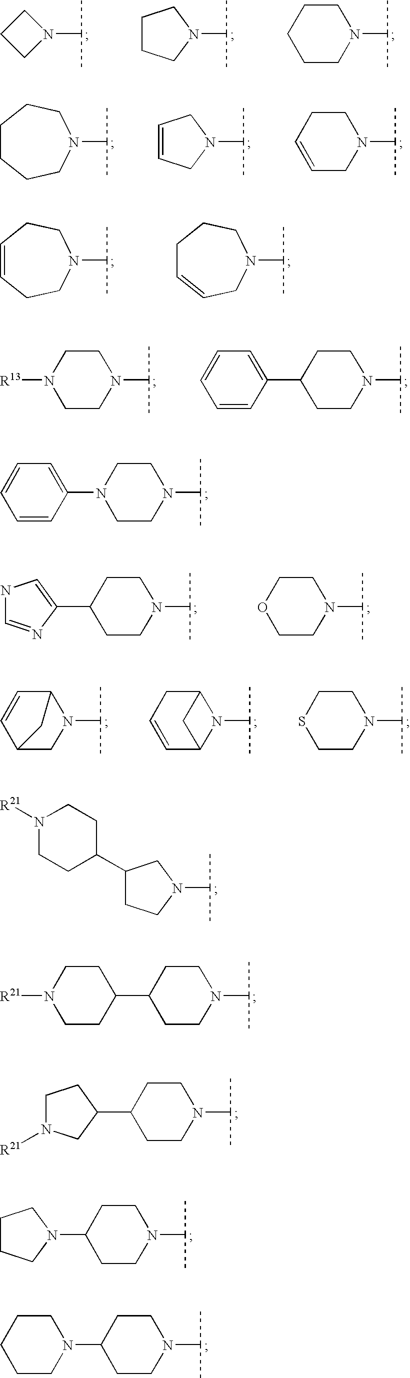 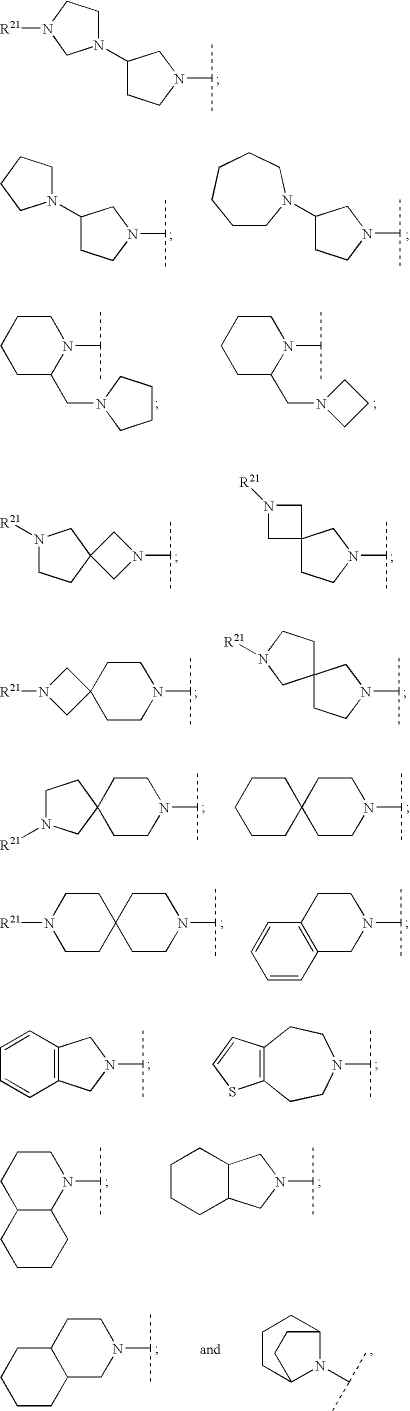
wherein one or more H atoms of the heterocycle formed by the group R¹R²N— may be replaced by identical or different groups R¹⁴, and

the heterocycle formed by the group R¹R²N— may be substituted by one or two, preferably one C₃₋₇-cycloalkyl group, while the cycloalkyl group may be mono- or polysubstituted by R²⁰, and

the ring linked to the heterocycle formed by the group R¹R²N— may be mono- or polysubstituted at one or more C atoms by R²⁰, and in the case of a phenyl ring may also additionally be monosubstituted by nitro and

wherein R¹³, R¹⁴, R²⁰, and R²¹ have the meanings given above and hereinafter.

If the heterocycle formed by the group R¹R²N— is substituted as specified by one or two cycloalkyl groups mono- or polysubstituted by R²⁰, the substituents R²⁰ independently of one another preferably denote C₁₋₄-alkyl, C₁₋₄-alkoxy-C₁₋₃-alkyl, hydroxy-C₁₋₃-alkyl, hydroxy, fluorine, chlorine, bromine, or CF₃, particularly hydroxy.

Most particularly preferably the group 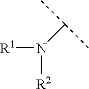
is therefore defined according to one of the following partial formulae 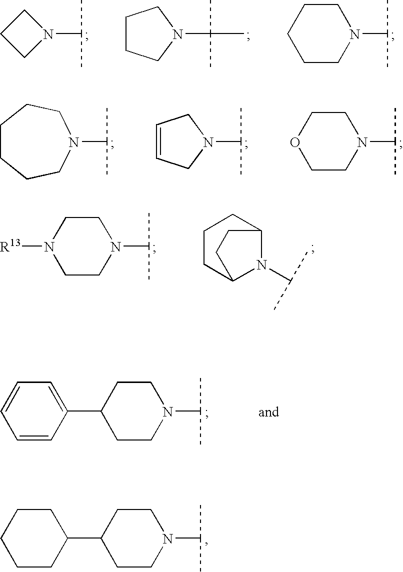
particularly 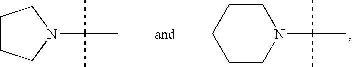
where R¹³ has the meanings given above and hereinafter, and the heterocycle formed by the group R¹R²N— may be substituted by C₃₋₆-cycloalkyl, hydroxy-C₃₋₆-cycloalkyl, or (hydroxy-C₃₋₆-cycloalkyl)-C₁₋₃-alkyl, and the heterocycle formed by the group R¹R²N— may be mono-, di- or trisubstituted by identical or different radicals R¹⁴. The substituents R¹⁴ preferably denote independently of one another F, Cl, Br, OH, C₁₋₄-alkyl, C₁₋₄-alkoxy, C₁₋₄-alkoxy-C₁₋₃-alkyl, hydroxy-C₁₋₄-alkyl or CF₃, particularly hydroxy, C₁₋₃-alkyl, CF₃, or hydroxy-C₁₋₃-alkyl.

If the partial formulae shown above are substituted as specified, the following definitions of the group R¹R²N— are particularly preferred: hydroxypyrrolidinyl, hydroxypiperidinyl, 3,4-dihydroxypyrrolidinyl, 3,4-dihydroxypiperidinyl, 3,5-dihydroxypiperidinyl, (hydroxymethyl)pyrrolidinyl, (hydroxymethyl)piperidinyl, (hydroxymethyl)hydroxypyrrolidinyl, and (hydroxymethyl)hydroxypiperidinyl, while in the groups specified a hydroxymethyl group at the C atom may be mono- or disubstituted by methyl, while two methyl substituents may be joined together, forming a cyclopropyl group, and while in one or two hydroxy groups the H atom may be replaced by a methyl group, and the groups specified do not have any other substituents or have one or two substituents selected independently of one another from fluorine, hydroxy, C₁₋₃-alkyl, hydroxy-C₁₋₃-alkyl, and CF₃.

The following partial formulae are most particularly preferred definitions of the abovementioned heterocyclic group 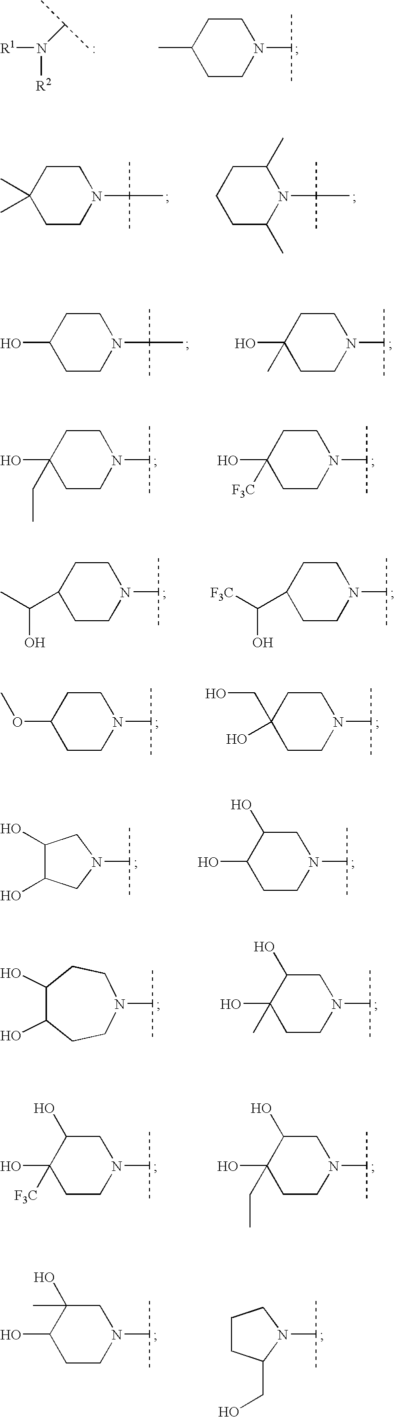 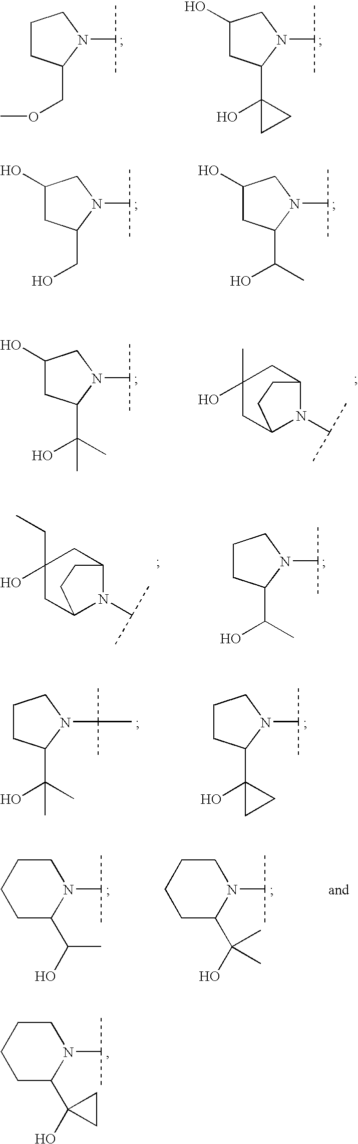
wherein the groups specified are not further substituted, or wherein methyl or ethyl groups may be mono-, di-, or trisubstituted by fluorine, and wherein one or more H atoms of the heterocycle formed by the group R¹R²N— which are bound to carbon may be substituted independently of one another by fluorine, chlorine, CN, CF₃, C₁₋₃-alkyl, or hydroxy-C₁₋₃-alkyl, particularly C₁₋₃-alkyl or CF₃, preferably methyl, ethyl, or CF₃.

Among the preferred and particularly preferred meanings of R¹R²N— listed above, the following definitions of the substituent R¹⁴ are preferred: F, Cl, Br, cyano, C₁₋₄-alkyl, C₂₋₄-alkenyl, C₂₋₄-alkynyl, C₃₋₇-cycloalkyl, C₃₋₇-cycloalkyl-C₁₋₃-alkyl, hydroxy, hydroxy-C₁₋₃-alkyl, C₁₋₄-alkoxy, ω-(C₁₋₄-alkoxy)-C₁₋₃-alkyl, C₁₋₄-alkyl-carbonyl, carboxy, C₁₋₄-alkoxycarbonyl, hydroxycarbonyl-C₁₋₃-alkyl, C₁₋₄-alkoxycarbonyl-C₁₋₃-alkyl, C₁₋₄-alkoxy-carbonylamino, C₁₋₄-alkoxy-carbonylamino-C₁₋₃-alkyl, amino, C₁₋₄-alkyl-amino, C₃₋₇-cycloalkyl-amino, N—(C₃₋₇-cycloalkyl)-N—(C₁₋₄-alkyl)-amino, di-(C₁₋₄-alkyl)-amino, cyclo-C₃₋₆-alkyleneimino, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, C₁₋₄-alkyl-aminocarbonyl, C₃₋₇-cycloalkyl-aminocarbonyl, N—(C₃₋₇-cycloalkyl)-N—(C₁₋₄-alkyl)-aminocarbonyl, di-(C₁₋₄-alkyl)-aminocarbonyl, pyridinyloxy, pyridinylamino, and pyridinyl-C₁₋₃-alkyl-amino.

Particularly preferred meanings of the substituent R¹⁴ are F, Cl, Br, C₁₋₄-alkyl, hydroxy, hydroxy-C₁₋₃-alkyl, C₁₋₄-alkoxy, ω-(C₁₋₄-alkoxy)-C₁₋₃-alkyl, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, and pyridylamino.

In the abovementioned preferred meanings of R¹⁴ in each case one or more C atoms may additionally be mono- or polysubstituted by F and/or in each case one or two C atoms may independently of one another additionally be monosubstituted by Cl or Br. Thus, preferred meanings of R¹⁴ also include for example —CF₃, —OCF₃, CF₃—CO—, and CF₃—CHOH—.

Most particularly preferred meanings of the substituent R¹⁴ are C₁₋₃-alkyl, hydroxy-C₁₋₃-alkyl, methoxymethyl, hydroxy, CF₃, and CF₃—CHOH—, particularly hydroxy, methyl, ethyl, CF₃, and hydroxymethyl.

If the bridge X is alkylenoxy or alkyleneimino as defined hereinbefore or hereinafter, the bridge is aligned such that the heteroatom is linked to the group Y. If the bridge X is an alkenylene group, the double bond is not directly attached to the group R¹R²N—.

The alkylene group in the group X representing alkylene, alkylenoxy, alkylimino, and alkenylene is unbranched without the substituents specified.

According to a first embodiment of the present invention, the bridge X denotes a C₁₋₆-alkylene bridge, particularly a C₂₋₄-alkylene bridge, which has one, two, three, or more, preferably one, two, or three substituents selected independently of one another from fluorine, chlorine, cyano, CF₃, hydroxy, C₁₋₄-alkyl, hydroxy-C₁₋₄-alkyl, C₃₋₆-cycloalkyl, and C₁₋₄-alkoxy, preferably selected from C₁₋₃-alkyl and cyclopropyl, while two alkyl substituents may be joined together forming a C₃₋₇-cycloalkyl group.

The bridge X is preferably a propylene bridge, which may be substituted as specified.

Preferably the alkylene bridge has 1, 2, or 3 substituents. Preferred substituents here are fluorine, chlorine, hydroxy, C₁₋₃-alkyl, and cyclopropyl, particularly C₁₋₃-alkyl and cyclopropyl, while two alkyl substituents may be joined together forming a C₃₋₆-cycloalkyl group.

Preferred definitions of the bridge X according to this first embodiment are selected from the group consisting of

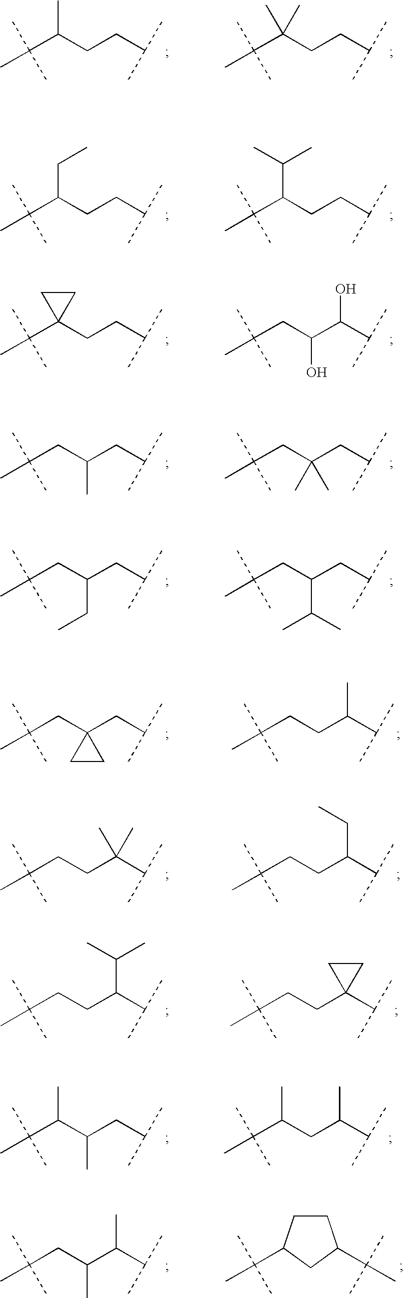

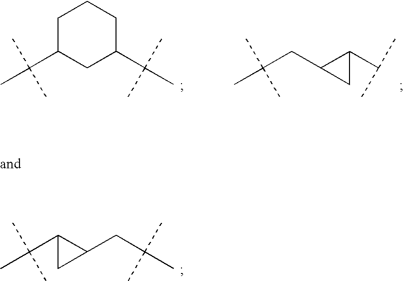

Particularly preferred definitions of the bridge X according to this first embodiment are selected from the group consisting of

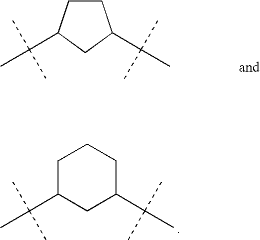

According to a second embodiment of the present invention, the bridge X denotes a C₂₋₄-alkylenoxy bridge, particularly a C₂₋₃-alkylenoxy bridge, which has 1, 2, 3, or more, preferably one, two, or three substituents selected independently of one another from fluorine, CF₃, C₁₋₄-alkyl, hydroxy-C₁₋₄-alkyl, and C₃₋₆-cycloalkyl, preferably selected from C₁₋₃-alkyl and cyclopropyl, while two alkyl substituents may be joined together forming a C₃₋₇-cycloalkyl group.

The bridge X is preferably an ethylenoxy bridge, which may be substituted as specified.

Preferably the alkylene unit has 1, 2, or 3 substituents. Preferred substituents here are fluorine, C₁₋₃-alkyl, and cyclopropyl, particularly methyl, ethyl, and isopropyl, while two alkyl substituents may be joined together forming a C₃₋₆-cycloalkyl group, particularly a cyclopropyl group.

Preferred definitions of the bridge X according to this second embodiment are selected from the group consisting of:

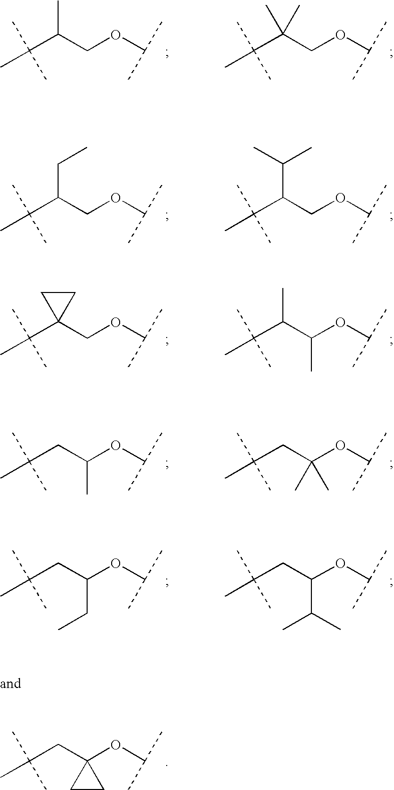

Particularly preferred definitions of the bridge X according to this second embodiment are selected from the group consisting of

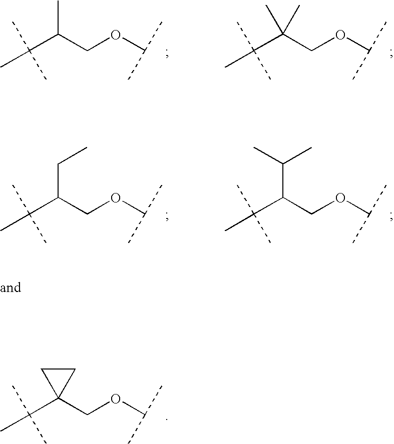

According to a third embodiment of the present invention, the bridge X denotes a C₂₋₄-alkyleneimino bridge, particularly a C₂₋₃-alkyleneimino bridge, wherein the imino group may be substituted by a C₁₋₄-alkyl group, and wherein the alkylene unit comprises 1, 2, 3, or more, preferably one, two, or three substituents selected independently of one another from fluorine, CF₃, C₁₋₄-alkyl, hydroxy-C₁₋₄-alkyl, and C₃₋₆-cycloalkyl, preferably selected from C₁₋₃-alkyl and cyclopropyl, while two alkyl groups may be joined together forming a C₃₋₇-cycloalkyl group or if an alkyl group is linked to the imino group may also be joined together to form a cyclo-C₄₋₆-alkyleneimino group.

The imino group is preferably unsubstituted or has a C₁₋₃-alkyl substituent, preferably a methyl group.

The bridge X is preferably an ethyleneimino bridge, which may be substituted as specified.

Preferably the alkylene unit has 1, 2, or 3 substituents. Preferred substituents here are fluorine, C₁₋₃-alkyl, and cyclopropyl, particularly methyl, ethyl, and isopropyl, while two alkyl substituents may be joined together forming a C₃₋₆-cycloalkyl group, particularly a cyclopropyl group, or if an alkyl group is linked to the imino group they may also be joined together forming a pyrrolidine or piperidine group.

Preferred definitions of the bridge X according to this second embodiment are selected from the group consisting of:

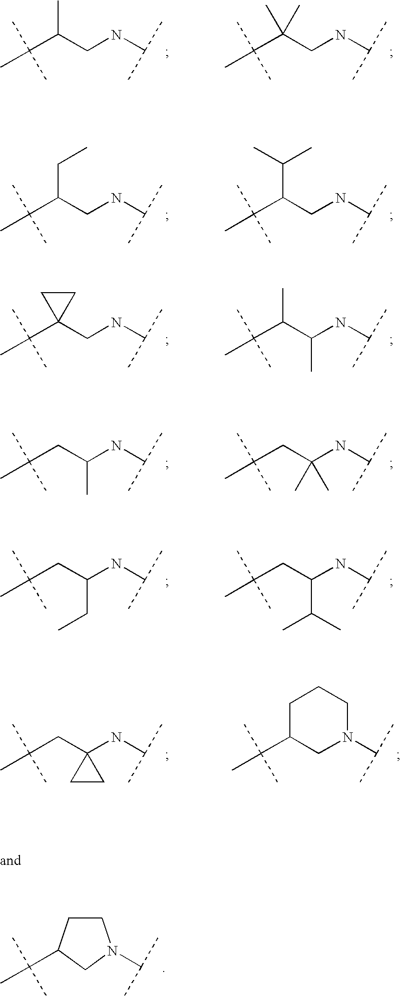

Particularly preferred definitions of the bridge X according to this third embodiment are selected from the group consisting of:

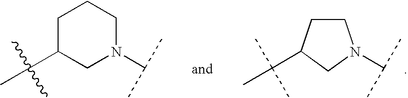

According to a fourth embodiment of the present invention, the bridge X denotes a C₃₋₆-alkenylene bridge, particularly a C₃₋₄-alkenylene bridge which is unsubstituted or comprises one, two, three or more, preferably one, two or three substituents selected independently of one another from fluorine, chlorine, CF₃, C₃₋₆-cycloalkyl, C₁₋₄-alkyl, and hydroxy-C₁₋₄-alkyl, preferably selected from C₁₋₃-alkyl and cyclopropyl, while two alkyl substituents may be joined together forming a C₃₋₇-cycloalkyl or C₅₋₇-cycloalkenyl group.

The bridge X is preferably a —CH₂—CH═CH— bridge, which may be substituted as specified.

Preferably the alkenylene bridge is unsubstituted or has 1, 2, or 3 substituents. Preferred substituents here are C₁₋₃-alkyl and cyclopropyl, while two alkyl substituents may be joined together forming a C₃₋₆-cycloalkyl or C₅₋₆-cycloalkenyl group.

Preferred definitions of the bridge X according to this fourth embodiment are selected from the group consisting of:

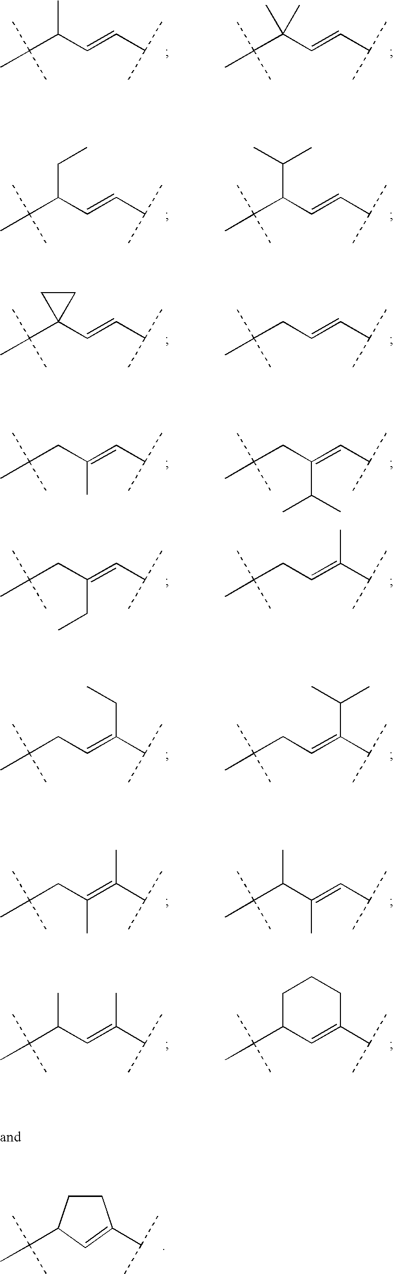

Particularly preferred definitions of the bridge X according to this fourth embodiment are selected from the group consisting of:

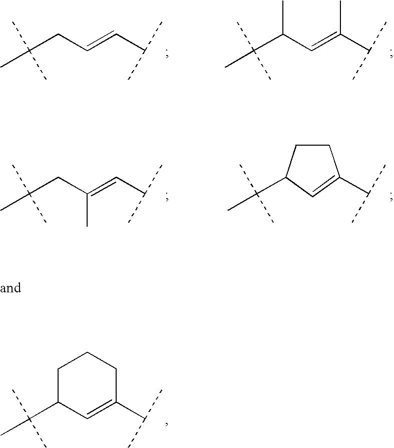

For X representing substituted alkenylene, only one of the two possible E/Z configurations is given above. Obviously, the other of the two E/Z configurations is also included according to the invention.

According to a fifth embodiment of the present invention the bridge X denotes a C₃₋₆-alkynylene bridge, particularly a C₃₋₄-alkynylene bridge which is unsubstituted or comprises one, two, three, or more, preferably one, two, or three substituents selected independently of one another from fluorine, chlorine, CF₃, C₃₋₆-cycloalkyl, C₁₋₄-alkyl, and hydroxy-C₁₋₄-alkyl, preferably selected from C₁₋₃-alkyl and cyclopropyl, while two alkyl substituents may be joined together forming a C₃₋₇-cycloalkyl group.

The bridge X is preferably a —CH₂—C≡C— bridge, which may be substituted as specified.

Preferably the alkynylene bridge is unsubstituted or has 1, 2, or 3 substituents. Preferred substituents are C₁₋₃-alkyl and cyclopropyl, while two alkyl substituents may be joined together, forming a C₃₋₆-cycloalkyl group.

Preferred definitions of the bridge X according to this fifth embodiment are selected from the group consisting of:

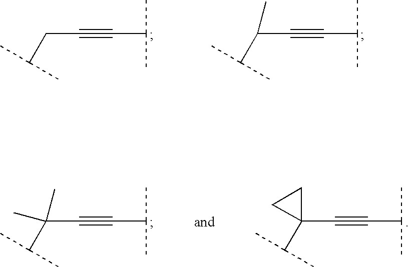

The bridge W preferably denotes a single bond or ethylene, particularly preferably a single bond.

The bridge Z preferably denotes a single bond or ethylene, which may have one or two methyl substituents, which may be joined together forming a cyclopropyl group. Particularly preferably, Z denotes a single bond.

The group Y preferably has a meaning selected from among the bivalent cyclic groups phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, chromanyl, chromen-4-onyl, benzothienyl, or benzofuranyl, particularly preferably phenyl, pyridinyl, pyrimidinyl, pyrazinyl, pyridazinyl, chromanyl, or chromen-4-onyl, while the abovementioned cyclic groups may be mono- or polysubstituted at one or more C atoms with identical or different groups R²⁰, or in the case of a phenyl ring may also additionally be monosubstituted by nitro, and/or may be substituted by R²¹ at one or more N atoms.

If the group Y is a 6-membered cyclic or heterocyclic group, the bridges X and Z are preferably attached to the group Y in the para position.

Particularly preferably the definition of the group Y is selected from among the bivalent cyclic groups: 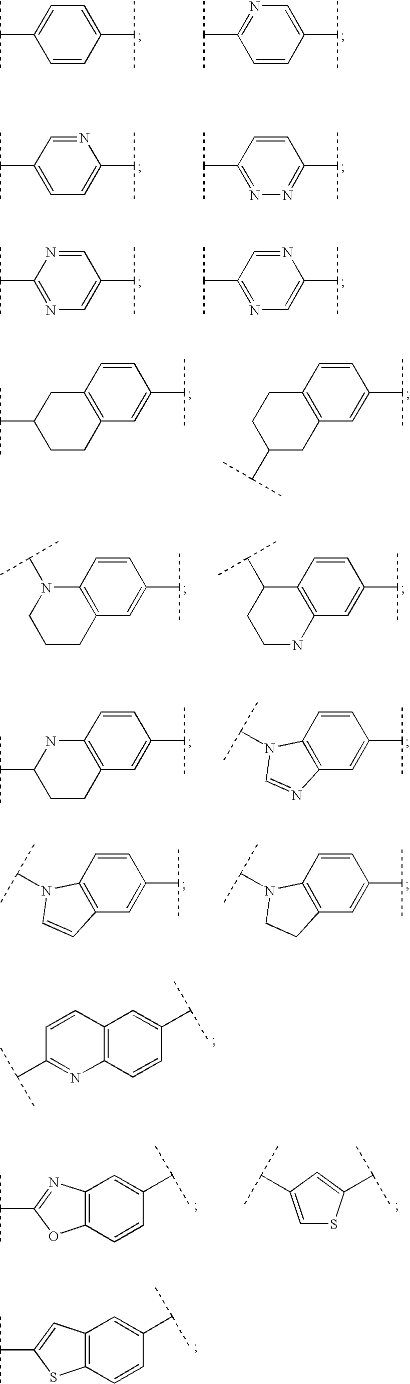 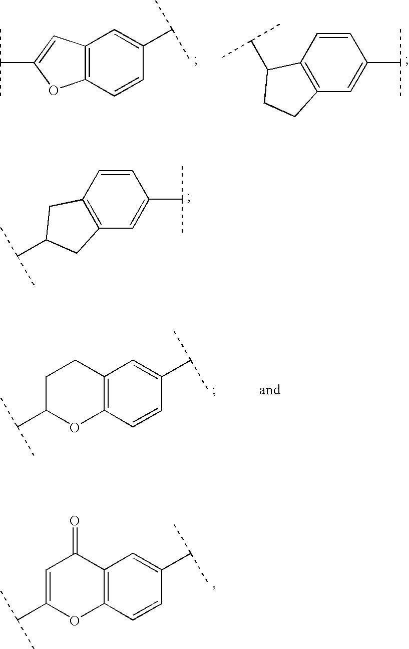
and in particular Y has one of the following meanings: 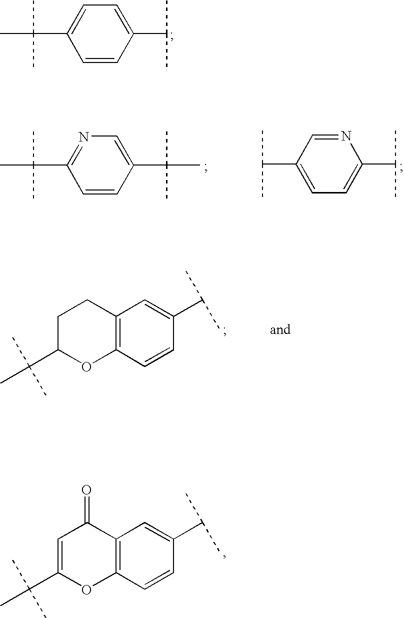
most particularly preferably Y has one of the following meanings: 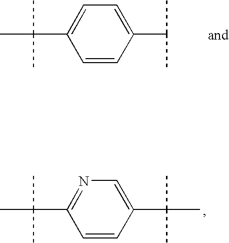
while the abovementioned cyclic groups may be mono- or polysubstituted at one or more C atoms by identical or different groups R²⁰, and in the case of a phenyl ring may also additionally be monosubstituted by nitro, and/or one or more NH groups may be substituted by R²¹.

The group Y is preferably unsubstituted or mono- or disubstituted.

Particularly preferred substituents R²⁰ of the group Y are selected from the group consisting of fluorine, chlorine, bromine, cyano, nitro, C₁₋₄-alkyl, C₂₋₆-alkenyl, hydroxy, ω-hydroxy-C₁₋₃-alkyl, C₁₋₄-alkoxy, trifluoromethyl, trifluoromethoxy, C₂₋₄-alkynyl, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, aminocarbonyl, C₁₋₄-alkyl-aminocarbonyl, and di-(C₁₋₄-alkyl)-aminocarbonyl.

Most particularly preferred substituents R²⁰ of the group Y are selected from among fluorine, chlorine, bromine, cyano, C₁₋₃-alkyl, C₁₋₃-alkoxy, C₁₋₄-alkoxycarbonyl, trifluoromethyl, trifluoromethoxy, or, in the case of a phenyl ring, nitro as well.

Most particularly preferably the group Y denotes substituted phenylene of the partial formula 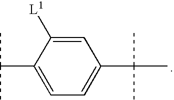
wherein L¹ has one of the meanings given hereinbefore for R²⁰, preferably F, Cl, Br, I, methyl, ethyl, ethenyl, ethynyl, CF₃, OCH₃, OCF₃, —CO—CH₃, —COOCH₃, CN or NO₂, or denotes H. Most particularly preferred meanings of the substituent L¹ are H, F, Cl, Br, methyl, ethyl, ethenyl, acetyl or methoxy, particularly H or methyl.

Preferably the group A is selected from among the bivalent cyclic groups phenyl, pyridinyl, pyrimidinyl, pyrazinyl or pyridazinyl, which may be mono- or polysubstituted at one or more C atoms by identical or different groups R²⁰, and in the case of a phenyl ring may also additionally be monosubstituted by nitro.

Most particularly preferably, A is one of the groups listed below: 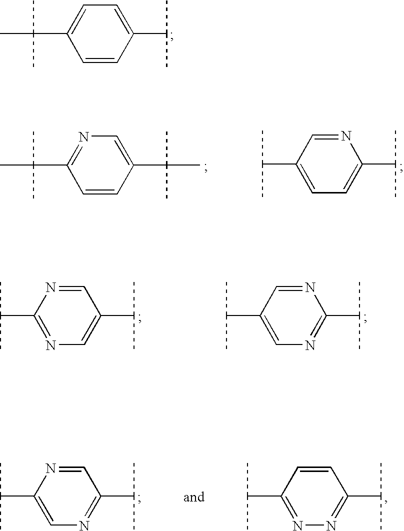
particularly 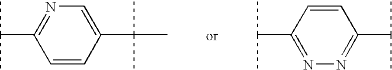
most particularly preferably 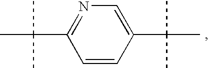
while the groups listed may be substituted as specified hereinbefore.

Particularly preferred substituents R²⁰ of the group A, independently of one another, are fluorine, chlorine, bromine, amino, CF₃, methoxy, and C₁₋₃-alkyl.

Preferably is the group A is unsubstituted or monosubstituted by R²⁰, as specified.

Preferred definitions of the group B according to a first preferred embodiment are selected from the group consisting of phenyl, pyridyl, thienyl, and furanyl. Particularly preferably the group B denotes phenyl. The group B defined as specified may be mono- or polysubstituted by identical or different groups R²⁰, a phenyl group may additionally also be monosubstituted by nitro. Preferably the group B is unsubstituted or mono-, di-, or trisubstituted, particularly unsubstituted or mono- or disubstituted. In the case of a monosubstitution, the substituent is preferably in the para position to the group A.

Preferred substituents R²⁰ of the group B are selected from the group consisting of fluorine, chlorine, bromine, cyano, nitro, C₁₋₄-alkyl, hydroxy, CHF₂, CHF₂—O—, hydroxy-C₁₋₃-alkyl, C₁₋₄-alkoxy, trifluoromethyl, trifluoromethoxy, C₂₋₄-alkynyl, carboxy, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, cyclo-C₃₋₆-alkyleneimino, aminocarbonyl, C₁₋₄-alkyl-aminocarbonyl, and di-(C₁₋₄-alkyl)-aminocarbonyl.

Particularly preferred substituents R²⁰ of the group B are selected from the group consisting of fluorine, chlorine, bromine, cyano, CF₃, C₁₋₃-alkyl, C₁₋₄-alkoxy, and trifluoromethoxy.

Most particularly preferred substituents R²⁰ of the group B are selected from the group consisting of chlorine, bromine, and methoxy.

According to a second embodiment the definition of the group B is preferably selected from C₁₋₆-alkyl, C₂₋₆-alkenyl, C₂₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, and C₃₋₇-cycloalkyl-C₁₋₃-alkynyl, while one or more C atoms in the groups mentioned for B hereinbefore may be mono- or polysubstituted by fluorine. In the cyclic groups according to the abovementioned embodiment, one or more C atoms may be substituted by R²⁰.

Particularly preferred according to this embodiment are the groups C₃₋₆-alkyl, C₃₋₆-alkenyl, C₃₋₆-alkynyl, cyclopentyl, cyclopentenyl, cyclohexyl, cyclohexenyl, cycloheptyl, cycloheptenyl, cyclopentyl-C₁₋₃-alkyl, cyclopentenyl-C₁₋₃-alkyl, cyclohexyl-C₁₋₃-alkyl, cyclohexenyl-C₁₋₃-alkyl, cycloheptyl-C₁₋₃-alkyl, and cycloheptenyl-C₁₋₃-alkyl, while one or more C atoms in the groups mentioned for B hereinbefore may be mono- or polysubstituted by fluorine, and in cyclic groups one or more C atoms may be substituted by identical or different groups R²⁰.

Most particularly preferably, according to this second embodiment B denotes cyclohexenyl, which is unsubstituted or comprises 1, 2, or 3 identical or different substituents R²⁰, particularly methyl.

The following are preferred definitions of other substituents according to the invention:

Preferably the substituent R¹³ has one of the meanings given for R¹⁶. Particularly preferably R¹³ denotes H, C₁₋₄-alkyl, C₃₋₇-cycloalkyl, C₃₋₇-cycloalkyl-C₁₋₃-alkyl, ω-hydroxy-C₂₋₃-alkyl, or ω-(C₁₋₄-alkoxy)-C₂₋₃-alkyl. Most particularly preferably R¹³ denotes H or C₁₋₄-alkyl. The alkyl groups specified above may be monosubstituted by Cl or mono- or polysubstituted by F.

Preferred meanings of the substituent R¹⁵ are H, C₁₋₄-alkyl, C₃₋₇-cycloalkyl, and C₃₋₇-cycloalkyl-C₁₋₃-alkyl, while, as hereinbefore defined, in each case one or more C atoms may additionally be mono- or polysubstituted by F and/or in each case one or two C atoms may independently of one another additionally be monosubstituted by Cl or Br. Particularly preferably, R¹⁵ denotes H, CF₃, methyl, ethyl, propyl, or butyl.

The substituent R¹⁶ preferably denotes H, C₁₋₄-alkyl, C₃₋₇-cycloalkyl, C₃₋₇-cycloalkyl-C₁₋₃-alkyl, ω-hydroxy-C₂₋₃-alkyl, or ω-(C₁₋₄-alkoxy)-C₂₋₃-alkyl, while, as hereinbefore defined, in each case one or more C atoms may additionally be mono- or polysubstituted by F and/or in each case one or two C atoms may independently of one another additionally be monosubstituted by Cl or Br. Particularly preferably R¹⁶ denotes H, CF₃, C₁₋₃-alkyl, C₃₋₆-cycloalkyl, or C₃₋₆-cycloalkyl-C₁₋₃-alkyl.

Preferably the substituent R¹⁷ has one of the meanings given as preferred for R¹⁶ or denotes phenyl, phenyl-C₁₋₃-alkyl, pyridinyl, or C₁₋₄-alkylcarbonyl. Particularly preferably R¹⁷ has one of the meanings given as preferred for R¹⁶.

Preferably one or both of the substituents R¹⁸ and R¹⁹ independently of one another denote hydrogen or C₁₋₄-alkyl, particularly hydrogen.

The substituent R²⁰ preferably denotes halogen, hydroxy, cyano, C₁₋₄-alkyl, C₂₋₄-alkenyl, C₂₋₄-alkynyl, C₃₋₇-cycloalkyl, C₃₋₇-cycloalkyl-C₁₋₃-alkyl, hydroxy-C₁₋₄-alkyl, R²²-C₁₋₃-alkyl, or one of the meanings given as preferred for R²², while, as hereinbefore defined, in each case one or more C atoms may additionally be mono- or polysubstituted by F and/or in each case one or two C atoms independently of one another may additionally be monosubstituted by Cl or Br.

Particularly preferred definitions of the group R²⁰ are halogen, hydroxy, cyano, C₁₋₄-alkyl, C₃₋₇-cycloalkyl, C₁₋₃-alkylcarbonyl, and C₁₋₄-alkoxy, while, as hereinbefore defined, in each case one or more C atoms may additionally be mono- or polysubstituted by F and/or in each case one or two C atoms independently of one another may additionally be monosubstituted by Cl or Br. Most particularly preferably R²⁰ denotes F, Cl, Br, I, OH, cyano, methyl, difluoromethyl, trifluoromethyl, ethyl, n-propyl, isopropyl, acetyl, methoxy, difluoromethoxy, trifluoromethoxy, ethoxy, n-propoxy, or isopropoxy.

The substituent R²² preferably denotes C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonyl-amino, hydroxy-C₁₋₃-alkylaminocarbonyl, aminocarbonylamino, or C₁₋₄-alkylaminocarbonyl-amino, while, as hereinbefore defined, in each case one or more C atoms may additionally be mono- or polysubstituted by F and/or in each case one or two C atoms may independently of one another additionally be monosubstituted by Cl or Br. Most particularly preferred meanings of R²² are C₁₋₄-alkoxy, C₃-alkylcarbonyl, amino, C₁₋₄-alkylamino, or di-(C₁₋₄-alkyl)-amino, wherein one or more H atoms may be replaced by fluorine.

Preferred definitions of the group R²¹ are C₁₋₄-alkyl, C₁₋₄-alkylcarbonyl, C₁₋₄-alkylsulfonyl, —SO₂—NH₂, —SO₂—NH—C₁₋₃-alkyl, —SO₂—N(C₁₋₃-alkyl)₂, and cyclo-C₃₋₆-alkyleneimino-sulfonyl, while, as hereinbefore defined, in each case one or more C atoms may additionally be mono- or polysubstituted by F and/or in each case one or two C atoms independently of one another may additionally be monosubstituted by Cl or Br. Most particularly preferably R²¹ denotes C₁₋₄-alkyl or CF₃. Cy preferably denotes a C₃₋₇-cycloalkyl, particularly a C₃₋₆-cycloalkyl group, a C₅₋₇-cycloalkenyl group, pyrrolidinyl, piperidinyl, piperazinyl, morpholinyl, thiomorpholinyl, aryl, or heteroaryl, and the abovementioned cyclic groups may be mono- or polysubstituted at one or more C atoms by identical or different radicals R²⁰, or in the case of a phenyl group may also additionally be monosubstituted by nitro, and/or one or more NH groups may be substituted by R²¹. Most particularly preferred definitions of the group Cy are C₃₋₆-cycloalkyl, pyrrolidinyl, and piperidinyl, which may be substituted as specified.

The term aryl preferably denotes phenyl or naphthyl, particularly phenyl.

The term heteroaryl preferably includes pyridyl, indolyl, quinolinyl, and benzoxazolyl.

Preferred compounds according to the invention are those wherein one or more of the groups, radicals, substituents and/or indices have one of the meanings specified hereinbefore as being preferred.

Particularly preferred compounds according to the invention may be described by one of the general formulae IIa, IIb, IIc, and IId, most particularly preferably IIa and IIb: 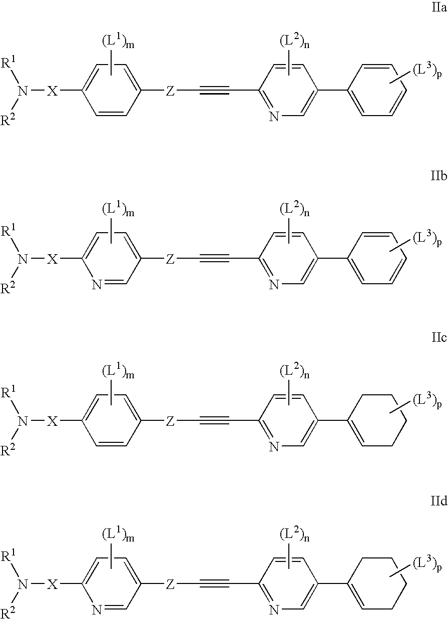
wherein:
R¹, R², X, and Z have one of the meanings given above;
L¹, L², and L³, independently of one another have one of the meanings given for R²⁰; and
m, n, and p independently of one another represent the values 0, 1, or 2, and p may also denote 3.

In particular, in formulae IIa, IIb, IIc, and IId, preferably IIa and IIb
Z denotes a single bond;
L¹ denotes fluorine, chlorine, bromine, cyano, C₁₋₃-alkyl, C₁₋₃-alkoxy, C₁₋₄-alkoxycarbonyl, trifluoromethyl, trifluoromethoxy, or nitro;
m denotes 0 or 1;
L² denotes fluorine, chlorine, bromine, CN, amino, CF₃, methoxy, or C₁₋₃-alkyl;
n denotes 0 or 1;
L³ independently of one another have a meaning selected from among fluorine, chlorine, bromine, cyano, nitro, C₁₋₄-alkyl, hydroxy, ω-hydroxy-C₁₋₃-alkyl, C₁₋₄-alkoxy, trifluoromethyl, trifluoromethoxy, C₂₋₄-alkynyl, carboxy, C₁₋₄-alkoxycarbonyl, ω-(C₁₋₄-alkoxy)-C₁₋₃-alkyl, C₁₋₄-alkoxy-carbonylamino, amino, C₁₋₄-alkyl-amino, di-(C₁₋₄-alkyl)-amino, cyclo-C₃₋₆-alkyleneimino, aminocarbonyl, C₁₋₄-alkyl-aminocarbonyl, or di-(C₁₋₄-alkyl)-aminocarbonyl, particularly preferably fluorine, chlorine, bromine, cyano, CF₃, C₁₋₃-alkyl, C₁₄₋-alkoxy and trifluoromethoxy, with the proviso that a phenyl ring may only be monosubstituted by nitro; and
p denotes 0, 1, 2, or 3, particularly 1 or 2.

Most particularly preferably, in the formulae IIa, IIb, IIc, and IId, particularly IIa and IIb,
R¹ and R² independently of one another denote C₁₋₄-alkyl, hydroxy-C₁₋₄-alkyl, C₃₋₅-alkenyl, C₃₋₅-alkynyl, C₃₋₇-cycloalkyl, hydroxy-C₃₋₇-cycloalkyl, dihydroxy-C₃₋₆-alkyl, C₃₋₇-cycloalkyl-C₁₋₃-alkyl, tetrahydropyran-3-yl, tetrahydropyran-4-yl, (hydroxy-C₃₋₇-cycloalkyl)-C₁₋₃-alkyl, ω-(C₁₋₄-alkoxy)-C₂₋₃-alkyl, pyridyl, or benzyl, while an alkyl, cycloalkyl, or cycloalkyl-alkyl group may additionally be mono- or disubstituted by hydroxy and/or hydroxy-C₁₋₃-alkyl, and/or mono- or polysubstituted by F or C₁₋₃-alkyl and/or monosubstituted by CF₃, Br, Cl, or CN, and one or both, preferably one of the groups R¹ and R² may also denote H, and phenyl and pyridyl rings may be mono- or polysubstituted by identical or different radicals R²⁰, while phenyl may also be monosubstituted by nitro, or
R¹ and R² are joined together and form, together with the N atom to which they are bound, a heterocyclic group which is selected from pyrrolidine, piperidine, 8-azabicyclo[3.2.1]octane, piperazine, wherein the free imine function is substituted by R¹³, and morpholine,
wherein one or more H atoms may be replaced by identical or different groups R¹⁴, and while the heterocyclic group defined hereinbefore may be substituted through a single bond with a carbo- or heterocyclic group Cy, while Cy is selected from the group consisting of C₃₋₇-cycloalkyl and cyclo-C₃₋₆-alkyleneimino, while Cy may be mono- or polysubstituted by identical or different radicals R²⁰, where R²⁰ is as hereinbefore defined and is preferably selected from fluorine, CF₃, C₁₋₃-alkyl, hydroxy-C₁₋₃-alkyl, and hydroxy;
R¹⁴ is selected from F, Cl, Br, C₁₋₄-alkyl, hydroxy, hydroxy-C₁₋₃-alkyl, C₁₋₄-alkoxy, ω-(C₁₋₄-alkoxy)-C₁₋₃-alkyl, amino-C₁₋₃-alkyl, C₁₋₄-alkyl-amino-C₁₋₃-alkyl, C₃₋₇-cycloalkyl-amino-C₁₋₃-alkyl, N—(C₃₋₇-cycloalkyl)-N—(C₁₋₄-alkyl)-amino-C₁₋₃-alkyl, di-(C₁₋₄-alkyl)-amino-C₁₋₃-alkyl, cyclo-C₃₋₆-alkyleneimino-C₁₋₃-alkyl, aminocarbonyl, and pyridylamino, while in the abovementioned meanings one or more C atoms may each additionally be mono- or polysubstituted by F and/or in each case one or two C atoms may independently of one another additionally be monosubstituted by Cl or Br;
X denotes a C₂₋₄-alkylene bridge which comprises 1, 2, or 3 substituents selected independently of one another from C₁₋₃-alkyl and cyclopropyl, while two alkyl substituents may be joined together forming a C₃₋₆-cycloalkyl group, or
a C₂₋₃-alkylenoxy bridge which comprises 1, 2, or 3 substituents selected independently of one another from C₁₋₃-alkyl and cyclopropyl, while two alkyl substituents may be joined together forming a C₃₋₆-cycloalkyl group, or
a C₂₋₃-alkyleneimino bridge, wherein the imino group may be substituted by a C₁₋₄-alkyl group, and wherein the alkylene unit comprises 1, 2, or 3 substituents selected independently of one another from C₁₋₃-alkyl and cyclopropyl, while two alkyl groups may be joined together forming a C₃₋₆-cycloalkyl group or if an alkyl group is linked to the imino group, they may also be joined together, forming a pyrrolidine or piperidine group, or
a C₃₋₄-alkenylene or C₃₋₄-alkynylene bridge which is unsubstituted or comprises 1, 2, or 3 substituents selected independently of one another from C₁₋₃-alkyl and cyclopropyl, while two alkyl substituents may be joined together forming a C₃₋₆-cycloalkyl or C₅₋₆-cycloalkenyl group.

The compounds listed in the experimental section, including the tautomers, the diastereomers, the enantiomers, the mixtures thereof and the salts thereof, are preferred according to the invention.

Some expressions used hereinbefore and below to describe the compounds according to the invention will now be defined more fully.

The term halogen denotes an atom selected from among F, Cl, Br, and I, particularly F, Cl, and Br.

The term C₁₋ₙ-alkyl, where n has a value of 3 to 8, denotes a saturated, branched or unbranched hydrocarbon group with 1 to n C atoms. Examples of such groups include methyl, ethyl, n-propyl, isopropyl, butyl, isobutyl, sec-butyl, tert-butyl, n-pentyl, isopentyl, neopentyl, tert-pentyl, n-hexyl, isohexyl, etc.

The term C₁₋ₙ-alkylene, where n may have a value of 1 to 8, denotes a saturated, branched or unbranched hydrocarbon bridge with 1 to n C atoms. Examples of such groups include methylene (—CH₂—), ethylene (—CH₂—CH₂—), 1-methylethylene (—CH(CH₃)—CH₂—), 1,1-dimethylethylene (—C(CH₃)₂—CH₂—), n-prop-1,3-ylene (—CH₂—CH₂—CH₂—), 1-methylprop-1,3-ylene (—CH(CH₃)—CH₂—CH₂—), 2-methylprop-1,3-ylene (—CH₂—CH(CH₃)—CH₂—), etc., as well as the corresponding mirror-symmetrical forms.

The term C₂₋ₙ-alkenyl, where n has a value of 3 to 6, denotes a branched or unbranched hydrocarbon group with 2 to n C atoms and at least one C═C-double bond. Examples of such groups include vinyl, 1-propenyl, 2-propenyl, isopropenyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 4-hexenyl, 5-hexenyl, etc.

The term C₂₋ₙ-alkynyl, where n has a value of 3 to 6, denotes a branched or unbranched hydrocarbon group with 2 to n C atoms and a C≡C triple bond. Examples of such groups include ethynyl, 1-propynyl, 2-propynyl, 1-butynyl, 2-butynyl, 3-butynyl, 2-methyl-1-propynyl, 1-pentynyl, 2-pentynyl, 3-pentynyl, 4-pentynyl, 3-methyl-2-butynyl, 1-hexynyl, 2-hexynyl, 3-hexynyl, 4-hexynyl, 5-hexynyl, etc.

The term C₁₋ₙ-alkoxy denotes a C₁₋ₙ-alkyl-O— group, wherein C₁₋ₙ-alkyl is defined as above. Examples of such groups include methoxy, ethoxy, n-propoxy, isopropoxy, n-butoxy, isobutoxy, sec-butoxy, tert-butoxy, n-pentoxy, isopentoxy, neopentoxy, tert-pentoxy, n-hexoxy, isohexoxy, etc.

The term C₁₋ₙ-alkylthio denotes a C₁₋ₙ-alkyl-S— group, wherein C₁₋ₙ-alkyl is defined as above. Examples of such groups include methylthio, ethylthio, n-propylthio, isopropylthio, n-butylthio, isobutylthio, sec-butylthio, tert-butylthio, n-pentylthio, isopentylthio, neopentylthio, tert-pentylthio, n-hexylthio, isohexylthio, etc.

The term C₁₋ₙ-alkylcarbonyl denotes a C₁₋ₙ-alkyl —C(═O)— group, wherein C₁₋ₙ-alkyl is defined as above. Examples of such groups include methylcarbonyl, ethylcarbonyl, n-propylcarbonyl, isopropylcarbonyl, n-butylcarbonyl, isobutylcarbonyl, sec-butylcarbonyl, tert-butylcarbonyl, n-pentylcarbonyl, isopentylcarbonyl, neopentylcarbonyl, tert-pentylcarbonyl, n-hexylcarbonyl, isohexylcarbonyl, etc.

The term C₃₋ₙ-cycloalkyl denotes a saturated mono-, bi-, tri- or spirocarbocyclic, preferably monocarbocyclic group with 3 to n C atoms. Examples of such groups include cyclopropyl, cyclobutyl, cyclopentyl, cyclohexyl, cycloheptyl, cyclooctyl, cyclononyl, cyclododecyl, bicyclo[3,2,1]octyl, spiro[4,5]decyl, norpinyl, norbonyl, norcaryl, adamantyl, etc.

The term C₅₋ₙ-cycloalkenyl denotes a monounsaturated mono-, bi-, tri- or spirocarbocyclic, preferably monocarboxylic group with 5 to n C atoms. Examples of such groups include cyclopentenyl, cyclohexenyl, cycloheptenyl, cyclooctenyl, cyclononenyl, etc.

The term C₃₋ₙ-cycloalkylcarbonyl denotes a C₃₋ₙ-cycloalkyl-C(═O) group, wherein C₃₋ₙ-cycloalkyl is as hereinbefore defined.

The term aryl denotes a carbocyclic, aromatic ring system, such as for example phenyl, biphenyl, naphthyl, anthracenyl, phenanthrenyl, fluorenyl, indenyl, pentalenyl, azulenyl, biphenylenyl, etc. A particularly preferred meaning of “aryl” is phenyl.

The term cyclo-C₃₋₆-alkyleneimino denotes a 4- to 7-membered ring which comprises 3 to 6 methylene units as well as an imino group, while the bond to the residue of the molecule is made via the imino group.

The term cyclo-C₃₋₆-alkyleneimino-carbonyl denotes a cyclo-C₃₋₆-alkyleneimino ring as hereinbefore defined which is linked to a carbonyl group via the imino group.

The term heteroaryl used in this application denotes a heterocyclic, aromatic ring system which comprises in addition to at least one C atom one or more heteroatoms selected from N, O, and/or S. Examples of such groups are furanyl, thiophenyl, pyrrolyl, oxazolyl, thiazolyl, imidazolyl, isoxazolyl, isothiazolyl, 1,2,3-triazolyl, 1,3,5-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinozilinyl, quinolinyl, isoquinolinyl, quinoxalinyl, naphthyridinyl, pteridinyl, carbazolyl, azepinyl, diazepinyl, acridinyl, etc. The term heteroaryl also comprises the partially hydrogenated heterocyclic, aromatic ring systems, particularly those listed above. Examples of such partially hydrogenated ring systems are 2,3-dihydrobenzofuranyl, pyrolinyl, pyrazolinyl, indolinyl, oxazolidinyl, oxazolinyl, oxazepinyl, etc. Particularly preferably heteroaryl denotes a heteroaromatic mono- or bicyclic ring system.

Terms such as C₃₋₇-cycloalkyl-C₁₋ₙ-alkyl, heteroaryl-C₁₋ₙ-alkyl, etc. refer to C₁₋ₙ-alkyl, as defined above, which is substituted with a C₃₋₇-cycloalkyl, aryl, or heteroaryl group.

Many of the terms given above may be used repeatedly in the definition of a formula or group and in each case have one of the meanings given above, independently of one another. Thus, for example, in the group di-C₁₋₄-alkyl-amino, the two alkyl groups may have the same or different meanings.

The term “unsaturated”, for example in “unsaturated carbocyclic group” or “unsaturated heterocyclic group”, as used particularly in the definition of the group Cy, comprises in addition to the mono- or polyunsaturated groups, the corresponding, totally unsaturated groups, but particularly the mono- and diunsaturated groups.

The term “optionally substituted” used in this application indicates that the group thus designated is either unsubstituted or mono- or polysubstituted by the substituents specified. If the group in question is polysubstituted, the substituents may be identical or different.

The style used hereinbefore and hereinafter, according to which in a cyclic group a bond of a substituent is shown towards the centre of this cyclic group, indicates unless otherwise stated that this substituent may be bound to any free position of the cyclic group carrying an H atom.

Thus, in the example 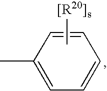
the substituent R²⁰ where s=1 may be bound to any of the free positions of the phenyl ring; where s=2 selected substituents R²⁰ may independently of one another be bound to different free positions of the phenyl ring.

The H atom of any carboxy group present or an H atom bound to an N atom (imino or amino group) may in each case be replaced by a group which can be cleaved in vivo. By a group which can be cleaved in vivo from an N atom is meant, for example, a hydroxy group, an acyl group such as the benzoyl or pyridinoyl group or a C₁₋₆-alkanoyl group such as the formyl, acetyl, propionyl, butanoyl, pentanoyl, or hexanoyl group, an allyloxycarbonyl group, a C₁₋₆-alkoxycarbonyl group such as the methoxycarbonyl, ethoxycarbonyl, propoxycarbonyl, isopropoxycarbonyl, butoxycarbonyl, tert-butoxycarbonyl, pentoxycarbonyl, hexyloxycarbonyl, octyloxycarbonyl, nonyloxycarbonyl, decyloxycarbonyl, undecyloxycarbonyl, dodecyloxycarbonyl, or hexadecyloxycarbonyl group, a phenyl-C₁₋₆-alkoxycarbonyl group such as the benzyloxycarbonyl, phenylethoxycarbonyl or phenylpropoxycarbonyl group, a C₁₋₃-alkylsulfonyl-C₂₋₄-alkoxycarbonyl, C₁₋₃-alkoxy-C₂₋₄-alkoxy-C₂₋₄-alkoxycarbonyl, or RₑCO—O—(R_{f}CR_{g})—O—CO— group wherein:
Rₑ denotes a C₁₋₈-alkyl, C₅₋₇-cycloalkyl, phenyl, or phenyl-C₁₋₃-alkyl group,
R_{f} denotes a hydrogen atom, a C₁₋₃-alkyl, C₅₋₇-cycloalkyl, or phenyl group, and
R_{g} denotes a hydrogen atom, a C₁₋₃-alkyl, or RₑCO—O—(R_{f}CR_{g})—O group wherein Rₑ to R_{g} are as hereinbefore defined,
   while the phthalimido group is an additional possibility for an amino group, and the abovementioned ester groups may also be used as a group which can be converted in vivo into a carboxy group.

The residues and substituents described above may be mono- or polysubstituted by fluorine as described. Preferred fluorinated alkyl groups are fluoromethyl, difluoromethyl, and trifluoromethyl. Preferred fluorinated alkoxy groups are fluoromethoxy, difluoromethoxy, and trifluoromethoxy. Preferred fluorinated alkylsulfinyl and alkylsulfonyl groups are trifluoromethylsulfinyl and trifluoromethylsulfonyl.

The compounds of general formula I according to the invention may have acid groups, predominantly carboxyl groups, and/or basic groups such as, e.g., amino functions. Compounds of general formula I may therefore be present as internal salts, as salts with pharmaceutically useable inorganic acids such as hydrochloric acid, sulfuric acid, phosphoric acid, sulfonic acid, or organic acids (such as for example maleic acid, fumaric acid, citric acid, tartaric acid, or acetic acid) or as salts with pharmaceutically useable bases such as alkali or alkaline earth metal hydroxides or carbonates, zinc or ammonium hydroxides or organic amines such as, e.g., diethylamine, triethylamine, triethanolamine inter alia.

The compounds according to the invention may be obtained using methods of synthesis which are known in principle. Preferably the compounds are obtained analogously to the methods of preparation explained more fully hereinafter.

The two reaction plans A and B that follow show the synthesis of the compounds A.5 and B.5 according to the invention, while R¹, R², X, Y, Z, W, A, and B have one of the meanings described hereinbefore. Hal denotes chlorine, bromine, or iodine, particularly bromine or iodine, particularly preferably iodine.

According to reaction plan A the halogen compound A.1 is reacted with the alkyne compound A.2 in a molar ratio of about 1.5:1 to 1:1.5 under a protective has atmosphere in the presence of a suitable palladium catalyst, a suitable base and copper (I) iodide in a suitable solvent.

A preferred amount of copper (I) iodide is in the range from 1 to 15 mol %, particularly 5 to 10 mol % based on the educt A. 1.

Suitable palladium catalysts are for example Pd(PPh₃)₄, Pd₂(dba)₃, Pd(OAc)₂, Pd(PPh₃)₂Cl₂, Pd(CH₃CN)₂Cl₂, and Pd(dppf)Cl₂. The palladium catalyst is preferably used in an amount from 1 to 15 mol %, particularly 5 to 10 mol % based on the educt A. 1.

Suitable bases are particularly amines, such as, for example, triethylamine or ethyldiisopropylamine, as well as cesium carbonate (Cs₂CO₃). The base is preferably used in an at least equimolar amount based on the educt A. 1, in excess or as the solvent. Moreover, suitable solvents are dimethylformamide or ether, such as for example tetrahydrofuran, including the mixtures thereof. The reaction takes place over a period of about 2 to 24 hours in a temperature range of about 20° C. to 90° C.

The alkyne compound A.3 obtained is reacted directly or after prior purification with methanesulfonic acid chloride to form the methanesulfonate derivative A.4. The reaction conditions required are known to the skilled man as such. Advantageous solvents are halogenated hydrocarbons, such as for example dichloromethane. Suitable reaction temperatures are usually in the range from 0° C. to 30° C.

The reaction solution containing the methanesulfonate derivative A.4 or the purified methanesulfonate derivative A.4, dissolved in a suitable solvent, is reacted with an amine H—NR¹R² to yield the end product A.5 and then optionally purified. If the amine H—NR¹R² has another primary or secondary amino function, this is advantageously provided with a protective group beforehand, which can be cleaved again after the reaction has ended, using methods known from the literature. The product thus obtained may for example be converted into the salt form by reaction with a corresponding acid. A preferred molar ratio of the derivative A.4 to the amine compound is in the range from 1.5:1 to 1:1.5. Suitable solvents are dimethylformamide or ether, such as for example tetrahydrofuran, including the mixtures thereof.

The reaction to form the product A.5 is advantageously carried out in a temperature range from about 20° C. to 90° C.

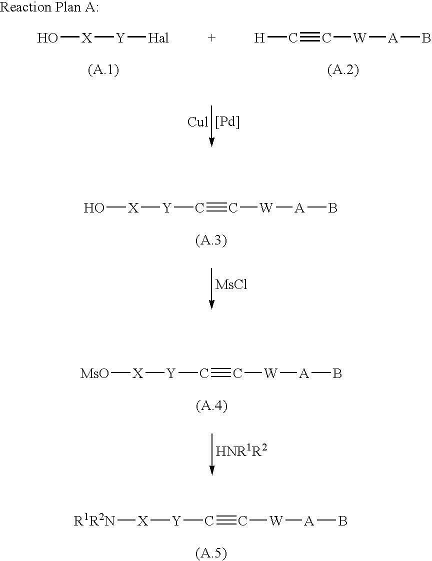

According to reaction plan B the halogen compound B.2 is reacted with the alkyne compound B.1 in a molar ratio of about 1.5:1 to 1:1.5 under a protective gas atmosphere in the presence of a suitable palladium catalyst, a suitable base and copper (I) iodide in a suitable solvent. Information on suitable reaction conditions, including catalysts, bases and solvents, may be found in the explanations of Reaction Plan A.

The alkyne compound B.3 obtained is reacted directly, or after prior purification, with methanesulfonic acid chloride to form the methanesulfonate derivative B.4. The reaction conditions to be respected can again be found in the remarks accompanying Reaction Plan A.

The reaction solution containing the methanesulfonate derivative B.4 or the purified methanesulfonate derivative B.4, dissolved in a suitable solvent, is reacted with an amine H—NR¹R² to form the end product B.5 and then optionally purified. Here again, the remarks concerning Reaction Plan A apply.

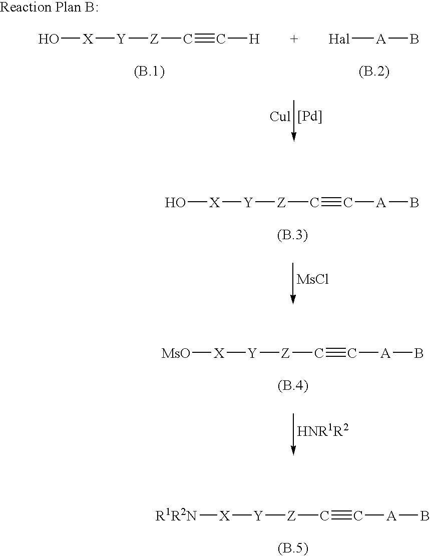

According to the other reaction plan C the halogen compound C.1 is reacted with the alkyne compound C.2 in a molar ratio of about 1.5:1 to 1:1.5 under a protective gas atmosphere in the presence of a suitable palladium catalyst, a suitable base, and copper (1) iodide in a suitable solvent to form the product C.3 directly. Information on suitable reaction conditions, including catalysts, bases and solvents, may be found in the explanatory remarks accompanying Reaction Plan A.

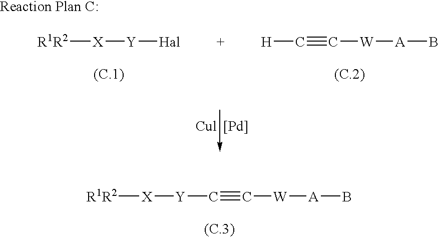

An alternative method of synthesis to this is shown in Reaction Plan D. According to this, the halogen compound D.2 is reacted with the alkyne compound D.1 in a molar ratio of about 1.5:1 to 1:1.5 under a protective gas atmosphere in the presence of a suitable palladium catalyst, a suitable base and copper (I) iodide in a suitable solvent to form the product D.3 directly. Once again, information on suitable reaction conditions, including catalysts, bases and solvents, may be found in the explanatory remarks accompanying Reaction Plan A.

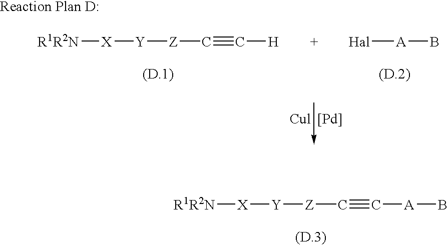

The reactions according to plans A, B, C, and D are particularly advantageously carried out with the corresponding iodine compounds A.1, B.2, C.1, and D.2. In the event that Hal denotes bromine in compounds A.1, B.2, C.1, or D.2, it is advantageous to convert it into the corresponding iodine compound beforehand. One particularly advantageous method is the Aryl-Finkelstein reaction (Artis Klapars and Stephen L. Buchwald, Copper-Catalyzed Halogen Exchange in Aryl Halides. An Aromatic Finkelstein Reaction, J. Am. Chem. Soc. (2002), 124 (50), pp. 14844-14845). Thus, for example, the halogen compound A.1, B.2, C.1 or D.2 may be reacted with sodium iodide in the presence of N,N′-dimethylethylenediamine and copper (D) iodide in a suitable solvent to form the corresponding iodine compound. An advantageous molar ratio of the halogen compound to sodium iodide is 1:1.8 to 1:2.3. N,N′-dimethylethylenediamine is advantageously used in a molar ratio of 10 to 30 mol % based on the halogen compound A.1, B.2, C.1, or D.2. Preferred amounts of copper (I) iodide are in the range from 5 to 20 mol % based on the halogen compound A.1, B.2, C.1, or D.2. A suitable solvent is for example 1,4-dioxane. Suitable reaction temperatures are in the range from about 20° C. to 110° C. The reaction is substantially complete after 2 to 72 hours.

The compounds according to the invention may be obtained using methods of synthesis which are known in principle. Preferably the compounds are obtained analogously to the methods of preparation explained more fully in the experimental section.

Stereoisomeric compounds of formula (I) may chiefly be separated by conventional methods. The diastereomers are separated on the basis of their different physico-chemical properties, e.g., by fractional crystallization from suitable solvents, by high pressure liquid or column chromatography, using chiral or preferably non-chiral stationary phases.

Racemates covered by general formula (I) may be separated, for example, by HPLC on suitable chiral stationary phases (e.g., Chiral AGP, CHIRALPAK® AD). Racemates which contain a basic or acidic function can also be separated via the diastereomeric, optically active salts which are produced on reacting with an optically active acid, for example, (+) or (−)-tartaric acid, (+) or (−)-diacetyl tartaric acid, (+) or (−)-monomethyl tartrate, or (+)-camphorsulfonic acid, or an optically active base, for example, with (R)-(+)-1-phenylethylamine, (S)-(−)-1-phenylethylamine, or (S)-brucine.

According to a conventional method of separating isomers, the racemate of a compound of formula (I) is reacted with one of the abovementioned optically active acids or bases in equimolar amounts in a solvent and the resulting crystalline, diastereomeric, optically active salts thereof are separated using their different solubilities. This reaction may be carried out in any type of solvent provided that it is sufficiently different in terms of the solubility of the salts. Preferably, methanol, ethanol or mixtures thereof, for example, in a ratio by volume of 50:50, are used. Then each of the optically active salts is dissolved in water, carefully neutralized with a base such as sodium carbonate or potassium carbonate, or with a suitable acid, e.g., with dilute hydrochloric acid or aqueous methanesulfonic acid and in this way the corresponding free compound is obtained in the (+) or (−) form.

The (R) or (S) enantiomer alone or a mixture of two optically active diastereomeric compounds of general formula (I) may also be obtained by performing the syntheses described above with a suitable reaction component in the (R) or (S) configuration.

As already mentioned, the compounds of formula (I) may be converted into the salts thereof, particularly for pharmaceutical use into the physiologically and pharmacologically acceptable salts thereof. These salts may be present on the one hand as physiologically and pharmacologically acceptable acid addition salts of the compounds of formula (I) with inorganic or organic acids. On the other hand, in the case of acidically bound hydrogen, the compound of formula (I) may also be converted by reaction with inorganic bases into physiologically and pharmacologically acceptable salts with alkali or alkaline earth metal cations as counter-ion. The acid addition salts may be prepared, for example, using hydrochloric acid, hydrobromic acid, sulfuric acid, phosphoric acid, methanesulfonic acid, ethanesulfonic acid, toluenesulfonic acid, benzenesulfonic acid, acetic acid, fumaric acid, succinic acid, lactic acid, citric acid, tartaric acid, or maleic acid. Moreover, mixtures of the abovementioned acids may be used. To prepare the alkali and alkaline earth metal salts of the compound of formula (I) with acidically bound hydrogen the alkali and alkaline earth metal hydroxides and hydrides are preferably used, while the hydroxides and hydrides of the alkali metals, particularly of sodium and potassium, are preferred and sodium and potassium hydroxide are most preferred.

The compounds according to the present invention, including the physiologically acceptable salts, are effective as antagonists of the MCH receptor, particularly the MCH-1 receptor, and exhibit good affinity in MCH receptor binding studies. Pharmacological test systems for MCH-antagonistic properties are described in the following experimental section.

As antagonists of the MCH receptor the compounds according to the invention are advantageously suitable as pharmaceutical active substances for the prevention and/or treatment of symptoms and/or diseases caused by MCH or causally connected with MCH in some other way. Generally the compounds according to the invention have low toxicity, they are well absorbed by oral route and have good intracerebral transitivity, particularly brain accessibility.

Therefore, MCH antagonists which contain at least one compound according to the invention are particularly suitable in mammals, such as, for example, rats, mice, guinea pigs, hares, dogs, cats, sheep, horses, pigs, cattle, monkeys, and humans, for the treatment and/or prevention of symptoms and/or diseases which are caused by MCH or are otherwise causally connected with MCH.

Diseases caused by MCH or otherwise causally connected with MCH are particularly metabolic disorders, such as, for example, obesity, and eating disorders, such as, for example, bulimia, including bulimia nervosa. The indication obesity includes in particular exogenic obesity, hyperinsulinemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity. This range of indications also includes cachexia, anorexia, and hyperphagia.

Compounds according to the invention may be particularly suitable for reducing hunger, curbing appetite, controlling eating behavior and/or inducing a feeling of satiation.

In addition, the diseases caused by MCH or otherwise causally connected with MCH also include hyperlipidemia, cellulitis, fatty accumulation, malignant mastocytosis, systemic mastocytosis, emotional disorders, affectivity disorders, depression, anxiety states, reproductive disorders, sexual disorders, memory disorders, epilepsy, forms of dementia, and hormonal disorders.

Compounds according to the invention are also suitable as active substances for the prevention and/or treatment of other illnesses and/or disorders, particularly those which accompany obesity, such as, for example, diabetes, diabetes mellitus, particularly type II diabetes, hyperglycemia, particularly chronic hyperglycemia, complications of diabetes including diabetic retinopathy, diabetic neuropathy, diabetic nephropathy, etc., insulin resistance, pathological glucose tolerance, encephalorrhagia, cardiac insufficiency, cardiovascular diseases, particularly arteriosclerosis and high blood pressure, arthritis, and gonitis.

MCH antagonists and formulations according to the invention may advantageously be used in combination with a dietary therapy, such as, for example, a dietary diabetes treatment, and exercise.

Another range of indications for which the compounds according to the invention are advantageously suitable is the prevention and/or treatment of micturition disorders, such as, for example, urinary incontinence, hyperactive bladder, urgency, nycturia, and enuresis, while the hyperactive bladder and urgency may or may not be connected with benign prostatic hyperplasia.

Generally speaking, the compounds according to the invention are potentially suitable for preventing and/or treating dependencies, such as, for example, alcohol and/or nicotine dependency, and/or withdrawal symptoms, such as, for example, weight gain in smokers coming off nicotine. By “dependency” is generally meant here an irresistible urge to take an addictive substance and/or to perform certain actions, particularly in order to either achieve a feeling of wellbeing or to eliminate negative emotions. In particular, the term “dependency” is used here to denote a dependency on an addictive substance. By “withdrawal symptoms” are meant here, in general, symptoms which occur or may occur when addictive substances are withdrawn from patients dependent on one or more such substances. The compounds according to the invention are potentially suitable particularly as active substances for reducing or ending tobacco consumption, for the treatment or prevention of a nicotine dependency and/or for the treatment or prevention of nicotine withdrawal symptoms, for reducing the craving for tobacco and/or nicotine and generally as an anti-smoking agent. The compounds according to the invention may also be useful for preventing or at least reducing the weight gain typically seen when smokers are coming off nicotine. The substances may also be suitable as active substances which prevent or at least reduce the craving for and/or relapse into a dependency on addictive substances. The term addictive substances refers particularly but not exclusively to substances with a psycho-motor activity, such as narcotics or drugs, particularly alcohol, nicotine, cocaine, amphetamine, opiates, benzodiazepines, and barbiturates.

The dosage required to achieve such an effect is conveniently, by intravenous or subcutaneous route, 0.001 to 30 mg/kg of body weight, preferably 0.01 to 5 mg/kg of body weight, and by oral or nasal route or by inhalation, 0.01 to 50 mg/kg of body weight, preferably 0.1 to 30 mg/kg of body weight, in each case 1to 3× daily.

For this purpose, the compounds prepared according to the invention may be formulated, optionally in conjunction with other active substances as described hereinafter, together with one or more inert conventional carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose, or fatty substances such as hard fat or suitable mixtures thereof, to produce conventional galenic preparations such as plain or coated tablets, capsules, lozenges, powders, granules, solutions, emulsions, syrups, aerosols for inhalation, ointments, or suppositories.

In addition to pharmaceutical compositions, the invention also includes compositions containing at least one alkyne compound according to the invention and/or a salt according to the invention optionally together with one or more physiologically acceptable excipients. Such compositions may also be, for example, foodstuffs which may be solid or liquid, in which the compound according to the invention is incorporated.

For the above mentioned combinations it is possible to use as additional active substances particularly those which, for example, potentiate the therapeutic effect of an MCH antagonist according to the invention in terms of one of the indications mentioned above and/or which make it possible to reduce the dosage of an MCH antagonist according to the invention. Preferably one or more additional active substances are selected from among active substances for the treatment of diabetes, active substances for the treatment of diabetic complications, active substances for the treatment of obesity, preferably other than MCH antagonists, active substances for the treatment of high blood pressure, active substances for the treatment of hyperlipidemia, including arteriosclerosis, active substances for the treatment of dyslipidemia, including arteriosclerosis, active substances for the treatment of arthritis, active substances for the treatment of anxiety states, and active substances for the treatment of depression.

The abovementioned categories of active substances will now be explained in more detail by means of examples.

Examples of active substances for the treatment of diabetes are insulin sensitizers, insulin secretion accelerators, biguanides, insulins, α-glucosidase inhibitors, and β₃ adrenoreceptor agonists. Insulin sensitizers include glitazones, particularly pioglitazone and its salts (preferably hydrochloride), troglitazone, rosiglitazone and its salts (preferably maleate), JTT-501, GI-262570, MCC-555, YM-440, DRF-2593, BM-13-1258, KRP-297, R-119702, and GW-1929. Insulin secretion accelerators include sulfonylureas, such as, for example, tolbutamide, chloropropamide, tolazamide, acetohexamide, glyclopyramide and its ammonium salts, glibenclamide, gliclazide, and glimepiride. Further examples of insulin secretion accelerators are repaglinide, nateglinide, mitiglinide (KAD-1229), and JTT-608. Biguanides include metformin, buformin, and phenformin. Insulins include those obtained from animals, particularly cattle or pigs, semisynthetic human insulins which are synthesized enzymatically from insulin obtained from animals, human insulin obtained by genetic engineering, e.g., from Escherichia coli or yeasts. Moreover, the term insulin also includes insulin-zinc (containing 0.45 to 0.9 percent by weight of zinc) and protamine-insulin-zinc obtainable from zinc chloride, protamine sulfate, and insulin. Insulin may also be obtained from insulin fragments or derivatives (for example INS-1, etc.). Insulin may also include different kinds, e.g., with regard to the onset time and duration of effect (“ultra immediate action type”, “immediate action type”, “two phase type”, “intermediate type”, “prolonged action type”, etc.), which are selected depending on the pathological condition of the patient. α-Glucosidase inhibitors include acarbose, voglibose, miglitol, and emiglitate. β₃ Adrenoreceptor agonists include AJ-9677, BMS-196085, SB-226552, and AZ40140. Active substances for the treatment of diabetes other than those mentioned above include ergoset, pramlintide, leptin, and BAY-27-9955 as well as glycogen phosphorylase inhibitors, sorbitol dehydrogenase inhibitors, protein tyrosine phosphatase 1B inhibitors, dipeptidyl protease inhibitors, glipazide, and glyburide.

Active substances for the treatment of diabetic complications include, for example, aldose reductase inhibitors, glycation inhibitors and protein kinase C inhibitors, DPP-IV blockers, GLP-2 or GLP-2 analogues, and SGLT-2 inhibitors. Aldose reductase inhibitors are, for example, tolrestat, epalrestat, imirestat, zenarestat, SNK-860, zopolrestat, ARI-50i, AS-3201. An example of a glycation inhibitor is pimagedine. Protein Kinase C inhibitors are, for example, NGF, and LY-333531. DPP-IV blockers are, for example, LAF237 (Novartis), MK431 (Merck), as well as 815541, 823093, and 825964 (all GlaxoSmithKline). GLP-1 analogues are, for example, Liraglutide (NN2211) (Novo Nordisk), CJC-1131 (Conjuchem), and Exenatide (Amylin). SGLT-2 inhibitors are, for example, AVE-2268 (Aventis) and T-1095 (Tanabe, Johnson & Johnson). Active substances other than those mentioned above for the treatment of diabetic complications include alprostadil, thiapride hydrochloride, cilostazol, mexiletine hydrochloride, ethyl eicosapentate, memantine, and pimagedine (ALT-711).

Active substances for the treatment of obesity, preferably other than MCH antagonists, include lipase inhibitors and anorectics. A preferred example of a lipase inhibitor is orlistat. Examples of preferred anorectics are phentermine, mazindol, fluoxetine, sibutramine, baiamine, (S)-sibutramine, SR-141716, and NGD-95-1. Active substances other than those mentioned above for the treatment of obesity include lipstatin.

Moreover, for the purposes of this application, the active substance group of anti-obesity active substances also includes the anorectics, of which the β₃ agonists, thyromimetic active substances and NPY antagonists should be emphasized. The range of substances which may be considered as preferred anti-obesity or anorectic active substances is indicated by the following additional list, by way of example: phenylpropanolamine, ephedrine, pseudoephedrine, phentermine, a cholecystokinin-A (hereinafter referred to as CCK-A) agonist, a monoamine reuptake inhibitor (such as, for example, sibutramine), a sympathomimetic active substance, a serotonergic active substance (such as, for example, dexfenfluramine, fenfluramine, a 5-HT2C agonist such as BVT.933 or APD356, or duloxetine), a dopamine antagonist (such as, for example, bromocriptine or pramipexole), a melanocyte-stimulating hormone receptor agonist or mimetic, an analogue of melanocyte-stimulating hormone, a cannabinoid receptor antagonist (ACOMPLIA® (rimonabant)), an MCH antagonist, the OB protein (hereinafter referred to as leptin), a leptin analogue, a fatty acid synthase (FAS) antagonist, a leptin receptor agonist, a galanine antagonist, and a GI lipase inhibitor or reducer (such as, for example, orlistat). Other anorectics include bombesin agonists, dehydroepiandrosterone or its analogues, glucocorticoid receptor agonists and antagonists, orexin receptor antagonists, urocortin binding protein antagonists, agonists of the Glucagon-like Peptide-1 receptor, such as, for example, exendin, AC 2993, CJC-1131, IP10, or GRT0203Y, DPP-IV inhibitors and ciliary neurotrophic factors, such as, for example, axokines. In this context mention should also be made of the forms of therapy which produce weight loss by increasing the fatty acid oxidation in the peripheral tissue, such as, for example, inhibitors of acetyl-CoA-carboxylase.

Active substances for the treatment of high blood pressure include inhibitors of angiotensin converting enzyme, calcium antagonists, potassium channel openers, and angiotensin II antagonists. Inhibitors of angiotensin converting enzyme include captopril, enalapril, alacepril, delapril (hydrochloride), lisinopril, imidapril, benazepril, cilazapril, temocapril, trandolapril, and manidipine (hydrochloride). Examples of calcium antagonists are nifedipine, amlodipine, efonidipine, and nicardipine. Potassium channel openers include levcromakalim, L-27152, AL0671, and NIP-121. Angiotensin II antagonists include telmisartan, losartan, candesartan cilexetil, valsartan, irbesartan, CS-866, and E4177.

Active substances for the treatment of hyperlipidemia, including arteriosclerosis, include HMG-CoA reductase inhibitors and fibrate compounds. HMG-CoA reductase inhibitors include pravastatin, simvastatin, lovastatin, atorvastatin, fluvastatin, lipantil, cerivastatin, itavastatin, ZD-4522 and their salts. Fibrate compounds include bezafibrate, clinofibrate, clofibrate, and simfibrate.

Active substances for the treatment of dyslipidemia, including arteriosclerosis, include, e.g., medicaments which raise the HDL level, such as, e.g., nicotinic acid and derivatives and preparations thereof, such as, e.g., niaspan, as well as agonists of the nicotinic acid receptor.

Active substances for the treatment of arthritis include NSAIDs (non-steroidal anti-inflammatory drugs), particularly COX-2 inhibitors, such as, for example, meloxicam or ibuprofen.

Active substances for the treatment of anxiety states include chlordiazepoxide, diazepam, oxozolam, medazepam, cloxazolam, bromazepam, lorazepam, alprazolam, and fludiazepam.

Active substances for the treatment of depression include fluoxetine, fluvoxamine, imipramine, paroxetine, and sertraline. The dosage for these active substances is conveniently ⅕ of the lowest normal recommended dose up to 1/1 of the normal recommended dose.

In another embodiment, the invention also relates to the use of at least one alkyne compound according to the invention and/or a salt according to the invention for influencing the eating behavior of a mammal. This use is particularly based on the fact that compounds according to the invention may be suitable for reducing hunger, curbing appetite, controlling eating behavior and/or inducing a feeling of satiety. The eating behavior is advantageously influenced so as to reduce food intake. Therefore, the compounds according to the invention are advantageously used for reducing body weight. Another use according to the invention is the prevention of increases in body weight, for example, in people who had previously taken steps to lose weight and are interested in maintaining their lower body weight. According to this embodiment it is preferably a non-therapeutic use. Such a non-therapeutic use might be a cosmetic use, for example, to alter the external appearance, or an application to improve general health. The compounds according to the invention are preferably used non-therapeutically for mammals, particularly humans, not suffering from any diagnosed eating disorders, no diagnosed obesity, bulimia, diabetes and/or no diagnosed micturition disorders, particularly urinary incontinence. Preferably, the compounds according to the invention are suitable for non-therapeutic use in people whose BMI (body mass index), defined as their body weight in kilograms divided by their height (in meters) squared, is below a level of 30, particularly below 25.

The Examples that follow are intended to illustrate the invention.

Preliminary Remarks

As a rule, IR, ¹H-NMR, and/or mass spectra have been obtained for the compounds prepared. Unless otherwise stated the R_{f} values were determined using ready-made silica gel 60 TLC plates F₂₅₄ (E. Merck, Darmstadt, Item No. 1.05714) without chamber saturation. The R_{f} values obtained under the heading Alox were determined using ready-made aluminum oxide 60 TLC plates F₂₅₄ (E. Merck, Darmstadt, Item No. 1.05713) without chamber saturation. For chromatographic purification, silica gel made by Messrs Millipore (MATREX™, 35-70 my) or Alox (E. Merck, Darmstadt, standardized aluminum oxide 90, 63-200 μm, Item No.: 1.01097.9050) is used. The ratios specified for the eluants are based on units by volume of the solvents in question. The specified units by volume of NH₃ solutions relate to a concentrated solution of NH₃ in water. Unless otherwise stated the acid, base and salt solutions used for working up the reaction solutions are aqueous systems of the concentrations specified.

The HPLC data specified were measured under the parameters indicated below:

Analytical columns: Zorbax column (Agilent Technologies), SB (Stable Bond)—C18; 3.5 μm; 4.6×75 mm; column temperature: 30° C.; flow: 0.8 mL/min; injection volume: 5 μL; detection at 254 nm (methods A, B and F).

Analytical columns: Zorbax column (Agilent Technologies), Bonus RP C14; 3.5 μm; 4.6×75 mm; column temperature: 30° C.; flow: 0.8 mL/min; injection volume: 5 μL; detection at 254 nm (methods C, D and E)

| | | |
|---|---|---|
| | percent by | percent by |
| | volume of water | volume of acetonitrile |
| time (min) | (with 0.1% formic acid) | (with 0.1% formic acid) |

Method A:

| | | |
|---|---|---|
| 0 | 95 | 5 |
| 9 | 10 | 90 |
| 10 | 10 | 90 |
| 11 | 90 | 10 |

Method B:

| | | |
|---|---|---|
| 0 | 95 | 5 |
| 4 | 10 | 90 |
| 10 | 10 | 90 |
| 11 | 90 | 10 |

Method C:

| | | |
|---|---|---|
| 0 | 95 | 5 |
| 9 | 10 | 90 |
| 12 | 10 | 90 |
| 13 | 90 | 10 |

Method D:

| | | |
|---|---|---|
| 0 | 95 | 5 |
| 9 | 10 | 90 |
| 10 | 10 | 90 |
| 11 | 90 | 10 |

Method E:

| | | |
|---|---|---|
| 0 | 95 | 5 |
| 4 | 10 | 90 |
| 10 | 10 | 90 |
| 11 | 90 | 10 |

Method F:

| | | |
|---|---|---|
| 0 | 95 | 5 |
| 9 | 10 | 90 |
| 12 | 10 | 90 |
| 13 | 90 | 10 |

Preparative column: Zorbax column (Agilent Technologies), SB (Stable Bond)—C18; 3.5 μm; 30×100 mm; column temperature: ambient temperature; flow: 30 mL/min; detection at 254 nm. In preparative HPLC purification, as a rule the same gradients are used which were used when obtaining the analytical HPLC data.

The products are collected under mass control, the fractions containing the product are combined and freeze-dried.

Chromatographic purification with Hyperprep made by Messrs Thermohypersil, Darmstadt: Stationary phase HS C18; 8 μM (eluant A (water+0.15% HCOOH), eluant B (methanol)).

Temperatures are given in degrees Celsius (° C.); times are generally given in minutes (min), hours (h), or days (d). If there is no specific information as to the configuration, it is not clear whether there are pure enantiomers or whether partial or even total racemization has taken place.

The following abbreviations are used above and hereinafter:
AcOH acetic acid
CDI 1,1′-carbonydiimidazole
DCM dichloromethane
DIAD diisopropylazodicarboxylate
DIPE diusopropyl ether
DMF dimethylformamide
dppf 1,1′-bis(diphenylphosphino)ferrocene
EtOAc ethyl acetate
conc. concentrated
MeOH methanol
PE petroleum ether
RT ambient (room) temperature
TBAF tetrabutylammonium fluoride trihydrate
TBME tert-butylmethylether
THF tetrahydrofuran
→* denotes the bonding site of a group
   General Experimental Method I (Sonogashira Couplings)

Under an argon atmosphere, a suitable palladium catalyst (e.g., Pd(PPh₃)₄ (5 mol %), Pd(PPh₃)₂Cl₂ (5 mol %), Pd(CH₃CN)Cl₂ (5 mol %), or Pd(dppf)Cl₂ (5 or 10 mol %)), a suitable base (e.g., cesium carbonate (1.5 eq.) or triethylamine (1.5 eq.)), and CuI (5 or 10 mol %) are added successively to a solution of the aryl or heteroaryl iodide or bromide (1.0 eq.) and the alkyne (1.05 eq.) in THF or DMF. The reaction solution is stirred at RT to 90° C. for between 2 hours and 24 hours, filtered, and the solvent is eliminated in vacuo. Further purification is carried out by column chromatography or by purification using HPLC-MS.

General Experimental Method II (Bromine-Iodine Exchange)

NaI (2.0 eq.), N,N′-dimethylethylenediamine (0.2 eq.), and CuI (0.1 eq.) are added successively to a solution of the aryl or heteroaryl bromide (1.0 eq.) in 1,4-dioxane under argon. The reaction is stirred for 2 hours to 72 hours at RT to 110° C. and then diluted with NH₃. The aqueous phase is extracted with DCM, the organic phase is dried over magnesium sulfate (MgSO₄) and the solvent is eliminated in vacuo. If necessary, further purification is carried out by column chromatography.
Component 1: 5-(4-chlorophenyl)-2-ethynylpyridine 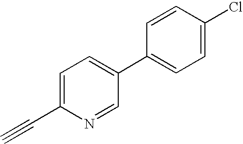
BS1a. 5-bromo-2-[(tert-butyldimethylsilanyl)ethynyl]pyridine

Under an argon atmosphere, 0.80 g (4.20 mmol) of CuI and 2.90 g (4.13 mmol) of bis(triphenylphosphane)palladium (II) chloride are added to a solution of 49.90 g (201.0 mmol) of 2,5-dibromopyridine and 43.0 mL (225.6 mmol) of tert-butylethynyldimethylsilane in 500 mL of dry THF and 120 mL of triethylamine at −7° C. and the mixture is stirred for 30 minutes at 0° C. The reaction mixture is stirred for a further 3.5 hours at RT, then filtered and the filtrate is evaporated down in vacuo. The residue is dissolved in 1 L of EtOAc, and the organic phase is washed with water and saturated NaCl solution, dried over sodium sulfate (Na₂SO₄), and evaporated down in vacuo. The crude product is reacted further without purification. Yield: 59.5 g (quant. yield); C₁₃H₁₈BrNSi (M=296.278); calc.: molpeak (M+H)⁺: 296/298 (Br); found: molpeak (M+H)⁺: 296/298 (Br); R_{f} value: 0.75 (silica gel, cyc/EtOAc 8: 1).

BS1b. 2-[(tert-butyldimethylsilanyl)ethynyl]-5-(4-chlorophenyl)pyridine

250 mL of MeOH, 220 mL of 2N sodium carbonate (Na₂CO₃) solution and 1.80 g (2.46 mmol) of PdCl₂(dppf) are added to a solution of 59.5 g (201.0 mmol) of 5-bromo-2-[(tert-butyldimethylsilanyl)ethynyl]pyridine and 36.5 g (233.4 mmol) of 4-chlorophenylboric acid in 600 mL of 1,4-dioxane and the mixture is refluxed for 1 hour. The reaction mixture is evaporated down in vacuo and diluted with EtOAc. The organic phase is washed with water and semisaturated sodium bicarbonate (NaHCO₃) solution, dried over sodium sulfate, and evaporated down in vacuo. The residue is purified by column chromatography (silica gel, cyc/EtOAc 9:1). Yield: 38.5 g (58% of theoretical); C₁₉H₂₂ClNSi (M=327.923); calc.: molpeak (M+H)⁺: 328/330 (Cl); found: molpeak (M+H)⁺: 328/330 (Cl); R_{f} value: 0.60 (silica gel, cyc/EtOAc 8:1).

BS1 c. 5-(4-chlorophenyl)-2-ethynylpyridine

43.66 g (156.0 mmol) of TBAF is added at RT to a solution of 46.50 g (142.0 mmol) of 2-[(tert-butyldimethylsilanyl)ethynyl]-5-(4-chlorophenyl)pyridine in 1 L of DCM and the mixture is stirred for 2 hours. The organic phase is washed with water, dried over sodium sulfate, and evaporated down in vacuo. The residue is stirred with DIPE, and the precipitate is filtered off and washed with PE. Yield: 26.0 g (86% of theoretical); C₁₃H₈ClN (M=213.662); calc.: molpeak (M+H)⁺: 214/216 (Cl); found: molpeak (M+H)⁺: 214/216 (Cl); R_{f} value: 0.30 (silica gel, cyc/EtOAc 4: 1).
Amine A1: 2,2,2-trifluoro-1-piperidin-4-ylethanol 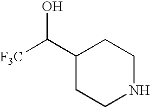
A1a. benzyl 4-(2,2,2-trifluoro-1-hydroxyethyl)piperidine-1-carboxylate

0.46 g (3.0 mmol) of cesium fluoride is added to a solution of 7.42 g (30.0 mmol) of benzyl 4-formylpiperidine-1-carboxylate in 120 mL of THF, the mixture is cooled to −10° C., 18.0 mL (36.0 mmol) of trimethyltrifluoromethylsilane (2.5 M in THF) is slowly added, and the mixture is stirred for 1.5 hours at −10° C. and 1.5 hours at RT. 120 mL of 1N HCl is added dropwise and the mixture is stirred for 1 hour. The organic phase is separated off and dried over sodium sulfate. After the desiccant and solvent have been eliminated, the residue is purified by chromatography (silica gel, cyc/EtOAc 4:1). Yield: 4.15 g (44% of theoretical); C₁₅H₁₈F₃NO₃ (M=317.304); calc.: molpeak (M+H)⁺: 318; found: molpeak (M+H)⁺: 318; HPLC-MS: 8.3 min (method A).

A1b. 2,2,2-trifluoro-1-piperidin4-ylethanol

A suspension of 3.11 g (9.80 mmol) of benzyl 4-(2,2,2-trifluoro-1-hydroxyethyl)piperidine-1-carboxylate and 300 mg 10% Pd/C in 30 mL of MeOH is hydrogenated at RT and 3 bar hydrogen pressure for 4 hours. The catalyst is filtered off and the filtrate is evaporated down in vacuo. Yield: 1.82 g (quant. yield); C₇H₁₃F₃NO (M=183.172); calc.: molpeak (M+H)⁺: 184; found: molpeak (M+H)⁺: 184; R_{f} value: 0.20 (silica gel, EtOAc/MeOH/NH₃ 50:50:5).
Amine A2:4-methyl-1-pyrrolidin-3-ylypiperidine 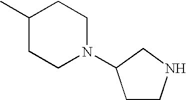
A2a. 1-(1-benzylpyrrolidin-3-yl)-4-methylpiperidine

13.0 g (61.5 mmol) of sodium triacetoxyborohydride and 2.37 mL (41.44 mmol) of AcOH are added to a solution of 6.0 mL (50.7 mmol) of 4-methylpiperidine and 8.14 mL (50.7 mmol) of 1-benzylpyrrolidin-3-one in 200 mL of THF and the reaction solution is stirred overnight at RT. Saturated sodium bicarbonate solution is added to the reaction mixture, which is exhaustively extracted with EtOAc and the combined organic phases are dried over magnesium sulfate. After the desiccant and solvent have been eliminated, the residue is purified by chromatography (silica gel, DCM/MeOH/NH₃ 90:10:1). Yield: 6.58 g (50% of theoretical); C₁₇H₂₆N₂ (M=258.402); calc.: molpeak (M+H)+: 259; found: molpeak (M+H)⁺: 259; R_{f} value: 0.45 (silica gel, DCM/MeOH/NH₃ 90:10:1).

A2b. 4-methyl-1-pyrrolidin-3-ylpiperidine

A solution of 6.58 g (25.46 mmol) of 1-(1-benzylpyrrolidin-3-yl)-4-methylpiperidine in 60 mL of MeOH is combined with 0.6 g 10% Pd/C and hydrogenated at RT and 4 bar until the theoretical amount of hydrogen has been taken up (4 hours). The catalyst is filtered off, washed with MeOH, and the solvent is evaporated down in vacuo. Yield: 3.09 g (72% of theoretical); C₁₀₂₀N₂ (M=168.279); calc.: molpeak (M+H)⁺: 169; found: molpeak (M+H)⁺: 69; retention time HPLC: 1.0 min (method A).

### EXAMPLE 1

### 5-(4-chlorophenyl)-2-{4-[3-(3.5-dimethylpiperidin-1-yl)cyclohexyl]phenylethynyl}-pyridine

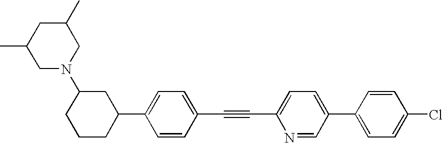 1a. 3-(4-bromophenyl)cyclohex-2-enone

Approximately 0.1 mL of dibromoethane is added to a suspension of 3.91 g (161 mmol) of magnesium chips and 3.80 g (16.1 mmol) of 1,4-dibromobenzene in 700 mL of diethyl ether and heated to 35° C. Then the remaining 1,4-dibromobenzene (34.2 g, 144.9 mmol) is slowly added dropwise to 400 mL of ether and the reaction is refluxed for 1 hour. 16.1 g (128 mmol) of 3-methoxycyclohex-2-enone in 25 mL of diethyl ether is slowly added dropwise and the reaction mixture is stirred for 1 hour at RT, before being added to 1000 mL of 1 M sulfuric acid. The aqueous phase is extracted three times with TBME. The organic phase is washed twice with 500 mL of water and dried over magnesium sulfate. After filtration through activated charcoal, the solvent is removed in vacuo. Further purification is carried out by column chromatography on silica gel (PE towards PE/EtOAc 7:3). Yield: 15.7 g (40.4% of theoretical); C₁₂H₁₁BrO (M=251.100); calc.: molpeak (M+H)⁺: 251/253 (Br); found: molpeak (M+H)⁺: 251/253 (Br); R_{f} value: 0.25 (silica gel, PE/EtOAc 8:2).

1b. 3-(4-bromophenyl)cyclohexanol

7.00 mL (7.00 mmol) of 1M lithium aluminum hydride solution in THF is added dropwise at −5° C. to a solution of 1.76 g (7.00 mmol) of 3-(4-bromophenyl)cyclohex-2-enone in 50 mL of THF. The reaction solution is heated to RT. After working up by the Fieser/Fieser method and filtration, the organic phase is dried over magnesium sulfate. After filtration through activated charcoal, the solvent is eliminated in vacuo and the crude product is reacted further without any further purification. Yield: 1.70 g (95.0% of theoretical); C₁₂H₁₅BrO (M=255.200); calc.: molpeak (M−H₂O ): 236/238 (Br); found: molpeak (M−H₂O ): 236/238 (Br); R_{f} value: 0.57 (silica gel, PE/EtOAc 6:4).

1c. 3-(4-iodophenyl)cyclohexanol

Prepared according to General Method II from 3-(4-bromophenyl)cyclohexanol (1.90 g, 7.45 mmol). Yield: 2.10 g (93.3% of theoretical); C₁₂H₁₅IO (M=302.151); calc.: molpeak (M)⁺: 302; found: molpeak (M)⁺: 302; R_{f} value: 0.60 (silica gel, PE/EtOAc 6:4).

1d. 3-{4-[5-(4-chlorophenyl)pyridin-2-ylethynyl]phenyl}cyclohexanol

Prepared according to General Method I from 3-(4-iodophenyl)cyclohexanol (755 mg, 2.50 mmol) and 5-(4-chlorophenyl)-2-ethynylpyridine (534 mg, 2.50 mmol). Yield: 520 mg (53.6% of theoretical); C₂₅H₂₂ClNO (M=387.901); calc.: molpeak (M+H)⁺: 388/390 (Cl); found: molpeak (M+H)⁺: 388/390 (Cl); R_{f} value: 0.07 (silica gel, PE/EtOAc 8:2).

1e. 3-{4-[5-(4-chlorophenyl)pyridin-2-ylethynyl]phenyl}cyclohexyl methanesulfonate

0.22 mL (2.70 mmol) of pyridine and 0.21 mL (2.68 mmol) of methanesulfonic acid chloride are added at 0° C. to a solution of 520 mg (1.34 mmol) of 3-{4-[5-(4-chlorophenyl) pyridin-2-ylethynyl]phenyl}cyclohexanol in 20 mL of DCM. The reaction solution is stirred for 2 hours at RT and a conversion of 10% is detected. Another 1.21 mL (15.44 mmol) of methanesulfonic acid chloride and 0.5 mL (6.22 mmol) of pyridine are added. After the reaction is complete, water is added and the organic phase is washed with dilute sodium bicarbonate solution. The organic phase is dried over magnesium sulfate and, after filtration through activated charcoal, the solvent is eliminated in vacuo. Further purification is carried out by column chromatography on silica gel (DCM/EtOAc 8:2). Yield: 300 mg (48.0% of theoretical); C₂₆H₂₄CINO₃S (M=465.992); calc.: molpeak (M+H)⁺: 466/468 (Cl); found: molpeak (M+H)⁺: 466/468 (Cl); R_{f} value: 0.90 (silica gel, DCM/EtOAc 9:1).

1f. 5-(4-chlorophenyl)-2-{4-[3-(3,5-dimethylpiperidin-1-yl)cyclohexyl]phenylethynyl}-pyridine

119 mg (1.05 mmol) of 3,5-dimethylpiperidine is added to a solution of 100 mg (0.21 mmol) of 3-{4-[5-(4-chlorophenyl)pyridin-2-ylethynyl]phenyl}cyclohexyl methanesulfonate in 2.0 mL of DMF and the reaction solution is stirred overnight at 60° C. and for a further 8 hours at 90° C. The reaction mixture is cooled to −10° C. and after filtration the residue is stirred with TBME. Yield: 8.5 mg (8.0% of theoretical); C₃₂H₃₅ClN₂ (M=483.086); calc.: molpeak (M+H)⁺: 483/485 (Cl); found: molpeak (M+H)⁺: 388/390 (Cl); R_{f} value: 0.47 (silica gel, EtOAc/MeOH/NH₃ 8:2).

### EXAMPLE 2

### 1′-{4-[5-(4-chlorophenyl)pyridin-2-ylethynyl]phenyl}-4-methyl-[1,3′]-bipiperidinyl

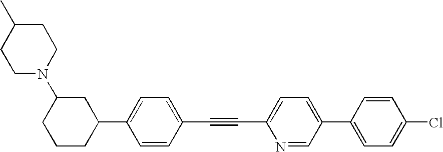 2a. 1′-benzyl-4-methyl-[1,3′]-bipiperidinyl

3.82 g (18.0 mmol) of NaBH(OAc)₃ is added to a solution of 1.78 mL (15.0 mmol) of 4-methylpiperidine and 3.66 g (15.0 mmol) of N-benzylpiperidin-3-one hydrochloride hydrate in 100 mL of THF and the solution is acidified slightly with glacial acetic acid. The reaction solution is stirred overnight at RT. The solvent is eliminated in vacuo and the residue is combined with saturated sodium bicarbonate solution. The aqueous phase is extracted twice with EtOAc and the organic phase is washed with saturated sodium bicarbonate solution, dried over magnesium sulfate, and the solvent is eliminated in vacuo. Further purification is carried out by column chromatography on silica gel (DCM after DCM/MeOH 8:2). Yield: 500 mg (12.2% of theoretical); C₁₈H₂₈N₂ (M=272.428); calc.: molpeak (M+H)⁺: 273; found: molpeak (M+H)⁺: 273; R_{f} value: 0.18 (silica gel, DCM/MeOH/NH₃ 9:1:0.1).

2b. 4-methyl-[1,3′]-bipiperidinyl

A solution of 500 mg (1.84 mmol) of 1′-benzyl-4-methyl-[1,3′]-bipiperidinyl in 25 mL of methanol is combined with 100 mg of 10% Pd(OH)₂ and hydrogenated in the autoclave at RT and 3 bar H₂ until the theoretical amount of hydrogen has been taken up. The catalyst is suction filtered and the filtrate concentrated by evaporation. The crude product is reacted further without any further purification. Yield: 0.35 g (99.4% of theoretical; content 95%); C₁₁H₂₂N₂ (M=182.306); calc.: molpeak (M+H)⁺: 183; found: molpeak (M+H)⁺: 183; R_{f}value: 0.08 (silica gel, EtOAc/MeOH/NH₃ 9:1:0.1).

2c. 1′-(4-bromophenyl)-4-methyl-[1,3′]-bipiperidinyl

566 mg (2.00 mmol) of 4-bromoiodobenzene, 7.6 mg (0.04 mmol) of CuI, 849 mg (4.00 mmol) of potassium phosphate, and 248 mg (4.00 mmol) of ethane-1,2-diol are added to a solution of 350 mg (1.92 mmol) of 4-methyl-[1,3′]-bipiperidinyl in 2.0 mL of isopropanol in an argon atmosphere. The reaction mixture is stirred overnight at 80° C. and then combined with 100 mL of EtOAc. The organic phase is extracted twice with 5% NH₃ solution and the aqueous phase is extracted once with 30 mL of EtOAc. The organic phase is dried over magnesium sulfate and the solvent is eliminated in vacuo. Further purification is carried out by column chromatography on silica gel (DCM towards DCM/MeOH/NH₃ 9:1:0.1). Yield: 100 mg (15.4% of theoretical); C₁₇H₂₅BrN₂ (M=337.298); R_{f} value: 0.68 (silica gel, DCM/MeOH/NH₃ 9:1:0.1); retention time HPLC: 4.52 min (method B).

2d. 1′-(4-iodophenyl)-4-methyl-[1,3′]-bipiperidinyl

Prepared according to General Method II from 1′-(4-bromophenyl)-4-methyl-[1,3′]-bipiperidinyl (100 mg, 0.30 mmol). Yield: 120 mg (100% of theoretical; 95% content); C₁₇H₂₅IN₂ (M=384.298); calc.: molpeak (M)⁺: 385; found: molpeak (M)⁺: 385; retention time HPLC: 4.64 min (method B).

2e. 1′-{4-[5-(4-chlorophenyl)pyridin-2-ylethynyl]phenyl}-4-methyl-[1,3 ′]-bipiperidinyl

Prepared according to General Method I from 1′-(4-iodophenyl)-4-methyl-[1,3′]-bipiperidinyl (120 mg, 0.30 mmol) and 5-(4-chlorophenyl)-2-ethynylpyridine (69 mg, 0.30 mmol). Yield: 50 mg (36.0% of theoretical); C₃₀H₃₂ClN₃ (M=470.048); calc.: molpeak (M+H)⁺: 470/472 (Cl); found: molpeak (M+H)⁺: 470/472 (Cl); R^{f} value: 0.28 (silica gel, DCM/MeOH 9:1).

### EXAMPLE 3.1

### 1-(3-{4-[5-(4-chlorophenyl)pyridin-2-ylethynyl]phenyl}cyclohex-2-enyl)-4-methylpiperidin-4-ol

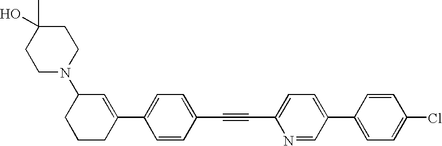 3.1 a. 3-(4-bromophenyl)cyclohex-2-enol

3.00 g (79.4 mmol) of sodium borohydride is added batchwise at 0° C. to a solution of 10.0 g (39.8 mmol) of 3-(4-bromophenyl)cyclohex-2-enone (see 1a) in 500 mL of MeOH. The reaction mixture is heated to RT and stirred for 1hour at RT. The reaction solution is added to 150 mL of 10% ammonium chloride solution, so that the temperature does not exceed 10° C. The aqueous phase is exhaustively extracted with DIPE and the combined organic extracts are washed three times with water. The organic phase is dried over magnesium sulfate and the solvent is eliminated in vacuo. The crude product is reacted further without any further purification. Yield: 9.5 g (94.2% of theoretical); C₁₂H₁₃BrO (M=253.135); calc.: molpeak (M+H−H₂O )⁺: 235/237 (Br); found: molpeak (M+H−H₂O )⁺: 235/237 (Br); R_{f} value: 0.65 (silica gel, DCM/MeOH 9:1).

3.1b. 3-(4-iodophenyl)cyclohex-2-enol

Prepared according to General Method II from 3-(4-bromophenyl)cyclohex-2-enol (10.8 g, 42.7 mmol). Yield: 9.80 g (76.5% of theoretical); C₁₂H₁₃IO (M=300.135); R_{f} value: 0.40 (silica gel, PE/EtOAc 6:4).

3.1 c. 1-[3-(4-iodophenyl)cyclohex-2-enyl]-4-methylpiperidin-4-ol

0.20 mL (2.13 mmol) of phosphorus tribromide in 5.0 mL of TBME is added at −10° C. to a solution of 600 mg (2.00 mmol) of 3-(4-iodophenyl)cyclohex-2-enol in 20 mL of TBME. The reaction solution is stirred for 2 hours at −10° C., combined with 50 mL ice water, and made alkaline with dilute sodium bicarbonate solution. The organic phase is dried over magnesium sulfate in the cold and 461 mg (4.00 mmol) of 4-methylpiperidin-4-ol is added immediately. The reaction mixture is heated to RT and then washed three times with 5% sodium carbonate solution. The organic phase is washed with water, dried over magnesium sulfate, and the solvent is eliminated in vacuo. The crude product is reacted further without any further purification. Yield: 500 mg (62.9% of theoretical); C₁₈H₂₄INO (M=397.294); calc.: molpeak (M+H)⁺: 398; found: molpeak (M+H)⁺: 398; retention time HPLC: 4.50 min (method B).

3.1d. 1-(3-{4-[5-(4-chlorophenyl)pyridin-2-ylethynyl]phenyl}cyclohex-2-enyl)-4-methylpiperidin-4-ol

Prepared according to General Method I from 1-[3-(4-iodophenyl)cyclohex-2-enyl]-4-methylpiperidin-4-ol (397 mg, 1.00 mmol) and 5-(4-chlorophenyl)-2-ethynylpyridine (214 mg, 1.00 mmol). Yield: 5.0 mg (1.0% of theoretical); C₃₁H₃₁ClN₂O (M=483.043); calc.: molpeak (M+H)⁺: 483/485 (Cl); found: molpeak (M+H)⁺: 483/485 (Cl); retention time HPLC: 8.4 min (method A).

### EXAMPLE 3.2

### 5-(4-chlorophenyl)-2-{4-[3-(3.5-dimethylpiperidin-1-yl)cyclohex-1-enyl]phenyl-ethynyl}pyridine

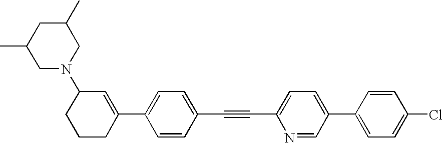 3.2a. 1-[3-(4-iodophenyl)cyclohex-2-enyl]-3,5-dimethylpiperidine

Analogously to Example 3.1c the product is obtained from 600 mg (2.00 mmol) of 3-(4-iodophenyl)cyclohex-2-enol and 1.20 g (10.6 mmol) of 3,5-dimethylpiperidine. Yield: 500 mg (63.2% of theoretical); C₁₉H₂₆IN (M=395.321); calc.: molpeak (M+H)⁺: 396; found: molpeak (M+H)⁺: 396; retention time HPLC: 5.4 min (method B).

3.2b. 5-(4-chlorophenyl)-2-{4-[3-(3,5-dimethylpiperidin-1-yl)cyclohex-1-enyl]phenyl-ethynyl}pyridine

Prepared according to General Method I from 1-[3-(4-iodophenyl)cyclohex-2-enyl]-3,5-dimethylpiperidine (395 mg, 1.00 mmol) and 5-(4-chlorophenyl)-2-ethynylpyridine (214 mg, 1.00 mmol). Yield: 310 mg (64% of theoretical); C₃₂H₃₃ClN₂ (M=481.071); calc.: molpeak (M+H)⁺: 481/483 (Cl); found: molpeak (M+H)^{+:} 481/483 (Cl); retention time HPLC: xx min (method A).

### EXAMPLE 3.3

### [(S)-1-(3-{4-[5-(4-chlorolphenyl)pyridin-2-ylethynyl]phenyl}cyclohex-2-enyl)pyrrolidin-2-yl]methanol

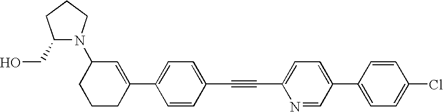 3.3a. {(S)-1-[3-(4-iodophenyl)cyclohex-2-enyl]pyrrolidin-2-yl}methanol

Analogously to Example 3.1c the product is obtained from 600 mg (2.00 mmol) of 3-(4-iodophenyl)cyclohex-2-enol and 1.16 mL (10.6 mmol) of (S)-(+)-2-hydroxymethylpyrrolidine. Yield: 500 mg (65.2% of theoretical); C₁₇H₂₂INO (M=383.267); calc.: molpeak (M+H)⁺: 384; found: molpeak (M+H)⁺: 384; retention time HPLC: 4.44 min (method B).

3.3b. [(S)-1-(3-{4-[5-(4-chlorophenyl)pyridin-2-ylethynyl]phenyl}cyclohex-2-enyl)pyrrolidin-2-yl]methanol

Prepared according to General Method I from {(S)-1-[3-(4-iodophenyl)cyclohex-2-enyl]pyrrolidin-2-yl}methanol (383 mg, 1.00 mmol) and 5-(4-chlorophenyl)-2-ethynylpyridine (214 mg, 1.00 mmol). Yield: 50 mg (11% of theoretical); C₃₀H₂₉ClN₂O (M=469.017); calc.: molpeak (M+H)⁺: 469/471 (Cl); found: molpeak (M+H)⁺: 469/471 (Cl); R_{f} value: 0.30 (silica gel, DCM/MeOH/NH₃ 9:1:0.1).

### EXAMPLE 4.1

### 1-(3-{4-[5-(4-chlorophenyl)pyridin-2-ylethynyl]phenyl}cyclopentyl)pilperidin-4-carboxylic acid amide

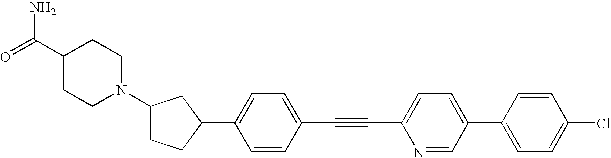 4.1a. 3-(4-bromophenyl)cyclopent-2-enone

Analogously to Example 1a the product is obtained from 40.0 g (169 mmol) of 1,4-dibromobenzene, 4.13 g (170 mmol) of magnesium chips, and 20.0 g (158 mmol) of 3-ethoxy-2-cyclopenten-1-one. Yield: 3.5 g (8.7% of theoretical); C₁₁H₉BrO (M=237.093); calc.: molpeak (M+H)⁺: 237/239 (Br); found: molpeak (M+H)⁺: 237/239 (Br); R_{f} value: 0.35 (silica gel, PE/EtOAc 6:4).

4.1b. 3-(4-bromophenyl)cyclopentanol

Analogously to Example 1b the product is obtained from 1.66 g (7.00 mmol) of 3-(4-bromophenyl)cyclopent-2-enone and 10.0 mL (10.0 mmol) of 1M lithium aluminum hydride solution in THF. Yield: 800 mg (47.4% of theoretical; content 60%; 40% debrominated product); C₁₁H₁₃BrO (M=241.124); calc.: molpeak (M+H)⁺: 240/242 (Br); found: molpeak (M+H)⁺: 240/242 (Br); R_{f} value: 0.6 (silica gel, DCM/MeOH 9:1).

4.1 c. 3-(4-iodophenyl)cyclopentanol

Prepared according to General Method II from 3-(4-bromophenyl)cyclopentanol (800 mg, 3.32 mmol). Yield: 1.00 g (52.3% of theoretical; content 50%); C₁₁H₁₃IO (M=288.125); calc.: molpeak (M)⁺: 288; found: molpeak (M)⁺: 288; retention time HPLC: 5.4 min (method B).

4.1d. 3-{4-[5-(4-chlorophenyl)pyridin-2-ylethynyl]phenyl}cyclopentanol

Prepared according to General Method I from 3-(4-iodophenyl)cyclopentanol (650 mg, 2.26 mmol) and 5-(4-chlorophenyl)-2-ethynylpyridine (483 mg, 2.26 mmol). Yield: 420 mg (49.7% of theoretical); C₂₄H₂₀CINO (M=373.874); calc.: molpeak (M+H)⁺: 374/376(Cl); found: molpeak (M+H)⁺: 374/376 (Cl); R_{f} value: 0.6 (silica gel, DCM/MeOH 9:1).

4.1e. methanesulfonate 3-{4-[5-(4-chlorophenyl)pyridin-2-ylethynyl]phenyl}cyclopentyl ester

Analogously to Example 1e the product is obtained from 400 mg (1.07 mmol) of 3-{4-[5-(4-chlorophenyl) pyridin-2-ylethynyl]phenyl}cyclopentanol, 1.28 mL (6.8 mmol) of methanesulfonic acid chloride, and 0.40 mL (5.00 mmol) of pyridine. Yield: 400 mg (82.7% of theoretical); C₂₅H₂₂ClNO₃S (M=451.966); calc.: molpeak (M+H)⁺: 452/454 (Cl); found: molpeak (M+H)⁺: 452/454 (Cl); R_{f} value: 0.4 (silica gel, PE/EtOAc 1:1).

4.1f. 1-(3-{4-[5-(4-chlorophenyl)pyridin-2-ylethynyl]phenyl}cyclopentyl)piperidin-4-carboxylic acid amide

83 mg (0.61 mmol) of isonipecotamide is added to a solution of 60 mg (0.13 mmol) of 3-{4-[5-(4-chlorophenyl)pyridin-2-ylethynyl]phenyl}cyclopentyl methanesulfonate in 2.0 mL of DMF and the reaction solution is stirred for 24 hours at 80° C. The reaction mixture is purified directly by HPLC-MS (water:acetonitrile:formic acid 95:5:0.1 towards 10:90:0.1). Yield: 6.5 mg (10.0% of theoretical); C₃₀H₃₀ClN₃O (M=484.032); calc.: molpeak (M+H)⁺: 484/486 (Cl); found: molpeak (M+H)⁺: 484/486 (Cl); retention time HPLC: 4.9 min (method B).

### EXAMPLE 4.2

### 1-(3-{4-[5-(4-chlorophenyl)pyridin-2-ylethynyl]phenyl}cyclopentyl)-4-methyl-piperidin-4-ol

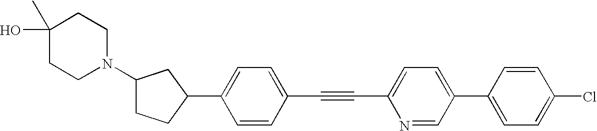

Analogously to 4.1f the product is obtained from 60 mg (0.13 mmol) of 3-{4-[5-(4-chlorophenyl)pyridin-2-ylethynyl]phenyl}cyclopentyl methanesulfonate and 45 mg (0.39 mmol) of 4-methylpiperidin-4-ol. Yield: 10 mg (16% of theoretical); C₃₀H₃₁ClN₂O (M=471.033); calc.: molpeak (M)⁺: 470/472 (Cl); found: molpeak (M)⁺: 470/472 (Cl); retention time HPLC: 5.64 min (method C).

### EXAMPLE 4.3

### 5-(4-chlorophenyl)-2-{4-[3-(4-methoxypiperidin-1-yl)cyclopentyl]phenyl- ethynyl}pyridine

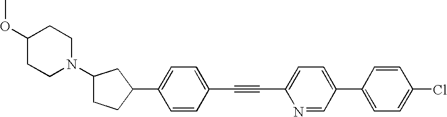

Analogously to 4.1f the product is obtained from 60 mg (0.13 mmol) of methanesulfonate 3-{4-[5-(4-chlorophenyl)pyridin-2-ylethynyl]phenyl}cyclopentyl and 75 mg (0.65 mmol) of 4-methoxypiperidine. Yield: 10 mg (16.3% of theoretical); C₃₀H₃₁ClN₂O (M=471.033); calc.: molpeak (M+H)⁺: 471/473 (Cl); found: molpeak (M+H)⁺: 471/473 (Cl); retention time HPLC: 5.4 min (method A).

### EXAMPLE 4.4

### [(S)-1-(3-{4-[5-(4-chlorophenyl)pyridin-2-ylethynyl]phenyl}cyclopentyl)-pyrrolidin-2-yl]methanol

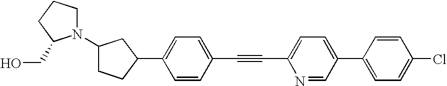

Analogously to 4.1f the product is obtained from 60 mg (0.13 mmol) of 3-{4-[5-(4-chlorophenyl)pyridin-2-ylethynyl]phenyl}cyclopentyl methanesulfonate and 71 μL (0.65 mmol) of (S)-(+)-2-hydroxymethylpyrrolidine. Yield: 6.5 mg (10.9% of theoretical); C₂₉H₂₉ClN₂O (M=457.006); calc.: molpeak (M+H)⁺: 457/459 (Cl); found: molpeak (M+H)⁺: 457/459 (Cl); retention time HPLC: 5.2 min (method A).

### EXAMPLE 5.1

### 1-(1-{4-[5-(4-chlorophenyl)pyridin-2-ylethynyl]phenyl}pyrrolidin-3-yl)-4-methylpiperidin-4-ol

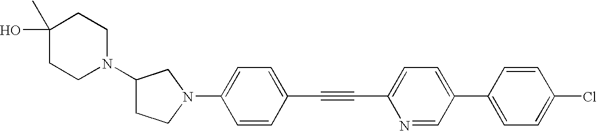 5.1a. 1-(4-bromophenyl)pyrrolidin-3-ol

Analogously to Example 2c the product is obtained from 871 mg (10.0 mmol) of 3-pyrrolidinone and 2.83 g (10.0 mmol) of 4-bromoiodobenzene. Yield: 1.30 g (53.7% of theoretical); C₁₀H₁₂BrNO (M=242.112); calc.: molpeak (M+H)⁺: 242/244 (Br); found: molpeak (M+H)⁺: 242/244 (Br); retention time HPLC: 7.77 min (method A).

5.1b. 1-(4-iodophenyl)pyrrolidin-3-ol

Prepared according to General Method II from 1-(4-bromophenyl)pyrrolidin-3-ol (1.30 g, 5.37 mmol). Yield: 1.30 g (83.7% of theoretical); C₁₀H₁₂INO (M=289.113); calc.: molpeak (M+H)⁺: 290; found: molpeak (M+H)⁺: 290; retention time HPLC: 8.14 min (method A).

5.1c. 1-{4-[5-(4-chlorophenyl)pyridin-2-ylethynyl]phenyl}pyrrolidin-3-ol

Prepared according to General Method I from 1-(4-iodophenyl)pyrrolidin-3-ol (1.30 g, 4.50 mmol) and 5-(4-chlorophenyl)-2-ethynylpyridine (1.03 g, 4.50 mmol). Yield: 1.40 g (83.1% of theoretical); C₂₃H₁₉ClN₂O (M=374.863); calc.: molpeak (M+H)⁺: 375/377 (Cl); found: molpeak (M+H)⁺: 375/377 (Cl); R_{f} value: 0.47 (silica gel, DCM/MeOH/NH₃ 9:1:0.1).

5.1d. 1-{4-[5-(4-chlorophenyl)pyridin-2-ylethynyl]phenyl}pyrrolidin-3-yl methanesulfonate

Analogously to Example 1e the product is obtained from 1.40 g (3.74 mmol) of 1-{4-[5-(4-chlorophenyl)pyridin-2-ylethynyl]phenyl}pyrrolidin-3-ol, 2.61 mL (33.6 mmol) of methanesulfonic acid chloride, and 3.62 mL (44.4 mmol) of pyridine. Yield: 900 mg (53.2% of theoretical); C₂₄H₂₁ClN₂O₃S (M=452.954); calc.: molpeak (M+H)⁺: 453/455 (Cl); found: molpeak (M+H)⁺: 453/455 (Cl); retention time HPLC: 6.26 min (method B).

5.1e. 1-(1-{4-[5-(4-chlorophenyl)pyridin-2-ylethynyl]phenyl}pyrrolidin-3-yl)-4-methyl-piperidin-4-ol

104 mg (0.90 mmol) of 4-methylpiperidin-4-ol are added to a solution of 82 mg (0.18 mmol) of 1-{4-[5-(4-chlorophenyl)pyridin-2-ylethynyl]phenyl}pyrrolidin-3-yl methanesulfonate in 2.0 mL of DMF and the reaction solution is stirred for 24 hours at 70° C. and for a further 60 hours at 60° C. The reaction mixture is diluted with 2.0 mL of water and, after filtration, the residue is applied to silica gel. Further purification is carried out by column chromatography on silica gel (DCM towards DCM/MeOH 9:1). Yield: 14.0 mg (16.5% of theoretical); C₂₉H₃₀ClN₃O (M=472.021); calc.: molpeak (M+H)^{+:} 472/474 (Cl); found: molpeak (M+H)^{+:} 472/474 (Cl); retention time HPLC: 5.03 min (method B).

### EXAMPLE 5.2

### 1-(1-{4-[5-(4-chlorophenyl)pyridin-2-ylethynyl]phenyl}pyrrolidin-3-yl)piperidin-4-carboxylic acid amide

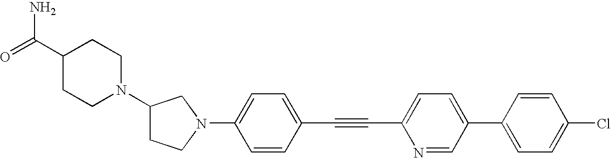

Analogously to 5.1e the product is obtained from 82 mg (0.18 mmol) of 1-{4-[5-(4-chlorophenyl)pyridin-2-ylethynyl]phenyl}pyrrolidin-3-yl methanesulfonate and 115 mg (0.90 mmol) of isonipecotamide. Yield: 22 mg (25.2% of theoretical); C₂₉H₂₉ClN₄O (M=485.020); calc.: molpeak (M+H)⁺: 485/487 (Cl); found: molpeak (M+H)⁺: 485/487 (Cl); retention time HPLC: 4.83 min (method B).

### EXAMPLE 5.3

### ((S)-1′-{4-[5-(4-chlorophenyl)pyridin-2-ylethynyl]phenyl}-[1,3′]-bipyrrolidinyl-2-yl)methanol

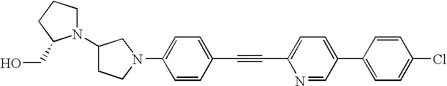

Analogously to 5.1e the product is obtained from 82 mg (0.18 mmol) of 1-{4-[5-(4-chlorophenyl)pyridin-2-ylethynyl]phenyl}pyrrolidin-3-yl methanesulfonate and 91 mg (0.90 mmol) of (S)-(+)-2-hydroxymethylpyrrolidine. Yield: 10 mg (12.1% of theoretical); C₂₈H₂₈ClN₃O (M=457.994); calc.: molpeak (M+H)⁺: 458/460 (Cl); found: molpeak (M+H)⁺: 458/460 (Cl); retention time HPLC: 5.08 min (method B).

### EXAMPLE 5.4

### 5-(4-chlorophenyl)-2-{4-[3-(4-methoxypiperidin-1-yl)pyrrolidin-1-yl]phenyl-ethynyl}pyridine

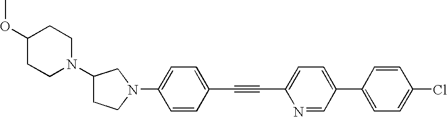

Analogously to 5.1e the product is obtained from 82 mg (0.18 mmol) of 1-{4-[5-(4-chlorophenyl)pyridin-2-ylethynyl]phenyl}pyrrolidin-3-yl methanesulfonate and 104 mg (0.90 mmol) of 4-methoxypiperidine. Yield: 32 mg (36.4% of theoretical); C₂₉H₃₀ClN₃O (M=472.021); calc.: molpeak (M+H)⁺: 472/474 (Cl); found: molpeak (M+H)⁺: 472/474 (Cl); retention time HPLC: 5.25 min (method B).

### EXAMPLE 5.5

### 5-(4-chlorophenyl)-2-{4-[3-(4-methylpiperidin-1-yl)pyrrolidin-1-yl]phenyl- ethynyl}pyridine

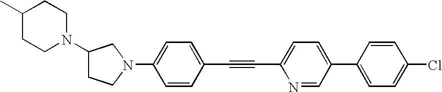

Analogously to 5.1e the product is obtained from 82 mg (0.18 mmol) of 1-{4-[5-(4-chlorophenyl)pyridin-2-ylethynyl]phenyl}pyrrolidin-3-yl methanesulfonate and 89 mg (0.90 mmol) of 4-methylpiperidine. Yield: 38 mg (46.3% of theoretical); C₂₉H₃₀ClN₃ (M=456.021); calc.: molpeak (M+H)⁺: 456/458 (Cl); found: molpeak (M+H)⁺: 456/458 (Cl); retention time HPLC: 5.50 min (method B).

### EXAMPLE 6.1

### (S)-1-(1-{-5-[5-(4-chlorophenyl)pyridin-2-ylethynyl]pyridin-2-yl}pyrrolidin-3-yl)-4-methylpiperidin-4-ol

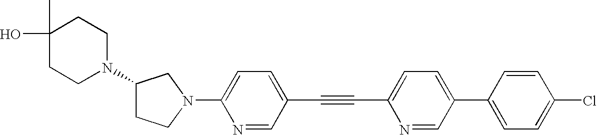 6.1a. (R)-1-(5-bromopyridin-2-yl)pyrrolidin-3-ol

5.00 g (56.2 mmol) of (R)-(+)-pyrrolidinole and 13.6 g (56.2 mmol) of 2,5-dibromopyridine are stirred for 1 hour in a melt at 140° C. The reaction mixture is cooled, combined with EtOAc, and the organic phase is washed with saturated sodium bicarbonate solution. The organic phase is dried over magnesium sulfate and the solvent is eliminated in vacuo. The residue is triturated in DIPE and dried after filtration. Further purification is carried out by column chromatography on silica gel (DCM/MeOH 9:1). Yield: 5.70 mg (41.7% of theoretical); CgH₁₁,BrN₂O (M=243.101); calc.: molpeak (M+H)⁺: 243/245 (Br); found: molpeak (M+H)⁺: 243/245 (Br); R_{f} value: 0.37 (silica gel, DCM/MeOH 9:1).

6.1b. (R)-1-(5-iodopyridin-2-yl)pyrrolidin-3-ol

Prepared according to General Method II from (R)-1-(5-bromopyridin-2-yl)pyrrolidin-3-ol (5.50 g, 22.6 mmol). Yield: 4.80 g (66.6% of theoretical; content: 91%); CgH₁₁IN₂O (M=290.101); calc.: molpeak (M)⁺: 291; found: molpeak (M)⁺: 291; retention time HPLC: 1.69 min (method A).

6.1c. (R)-1-{5-[5-(4-chlorophenyl)pyridin-2-ylethynyl]pyridin-2-yl}pyrrolidin-3-ol

Prepared according to General Method I from (R)-1-(5-iodopyridin-2-yl)pyrrolidin-3-ol (4.80 mg, 91% content, 15.1 mmol) and 5-(4-chlorophenyl)-2-ethynylpyridine (3.22 g, 15.1 mmol). Yield: 5.00 g (88.4% of theoretical); C₂₂H₁₈ClN₃O (M=375.851); calc.: molpeak (M+H)⁺: 376/378 (Cl); found: molpeak (M+H)⁺: 376/378 (Cl); retention time HPLC: 6.90 min (method A).

6.1d. (R)-methanesulfonate 1-{5-[5-(4-chlorophenyl)pyridin-2-ylethynyl]pyridin-2-yl}pyrrolidin-3-yl ester

Analogously to Example 1e the product is obtained from 920 mg (2.45 mmol) of (R)-1-{5-[5-(4-chlorophenyl)pyridin-2-ylethynyl]pyridin-2-yl}pyrrolidin-3-ol, 1.72 mL (22.0 mmol) of methanesulfonic acid chloride, 0.51 mL (3.68 mmol) of triethylamine, and 0.50 mL (6.13 mmol) of pyridine. Yield: 870 mg (62.6% of theoretical; content: 80%); C₂₃H₂₀ClN₃O₃S (M=453.942); calc.: molpeak (M+H)⁺: 454/456 (Cl); found: molpeak (M+H)⁺: 454/456 (Cl); retention time HPLC: 5.66 min (method B).

6.1e. (S)-1-(1-{5-[5-(4-chlorophenyl)pyridin-2-ylethynyl]pyridin-2-yl}pyrrolidin-3-yl)-4-methylpiperidin-4-ol

101 mg (0.88 mmol) of 4-methylpiperidin-4-ol is added to a solution of 100 mg (0.18 mmol, 60% content) of 1-{5-[5-(4-chlorophenyl)pyridin-2-ylethynyl]pyridin-2-yl}pyrrolidin-3-yl (R)-methanesulfonate in 1.0 mL of DMF and the reaction solution is stirred for 48 hours at 70° C. The reaction mixture is purified directly by column chromatography by HPLC-MS (water:acetonitrile:fornic acid 80:20:0.1 towards 75:25:0.1). Yield: 9.0 mg (10.8% of theoretical); C₂₈H₂₉ClN₄O (M=473.009); calc.: molpeak (M+H)⁺: 473/475 (Cl); found: molpeak (M+H)⁺: 473/475 (Cl); retention time HPLC: 4.94 min (method A).

### EXAMPLE 6.2

### (S)-5-(4-chlorophenyl)-2-{6-[3-(4-methylpiperidin-1-yl)pyrrolidin-1-yl]pyrid-3-ylethynyl}pyridine

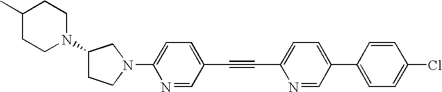

Analogously to 6.1e the product is obtained from 80 mg (0.14 mmol; 80% content) of 1-{5-[5-(4-chlorophenyl)pyridin-2-ylethynyl]pyridin-2-yl}pyrrolidin-3-yl (R)-methanesulfonate and 0.17 mL (1.41 mmol) of 4-methylpiperidine. Yield: 16 mg (24.8% of theoretical); C₂₈H₂₉ClN₄ (M=457.010); calc.: molpeak (M+H)⁺: 457/459 (Cl); found: molpeak (M+H)⁺: 457/459 (Cl); retention time HPLC: 4.85 min (method B).

### EXAMPLE 6.3

### (S)-1-(1-{5-[5-(4-chlorophenyl)pyridin-2-ylethynyl]pyridin-2-yl}pyrrolidin-3-yl)piperidin-4-carboxylic acid amide

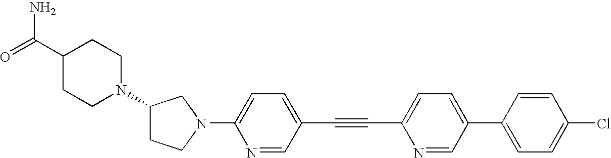

Analogously to 6.1e the product is obtained from 100 mg (0.18 mmol; 80% content) of 1-{5-[5-(4-chlorophenyl)pyridin-2-ylethynyl]pyridin-2-yl}pyrrolidin-3-yl (R)-methanesulfonate and 116 mg (0.88 mmol) of piperidin-4-carboxylic acid amide. Yield: 9.0 mg (10.5% of theoretical); C₂₈H₂₈ClN₅O (M=486.008); calc.: molpeak (M+H)⁺: 486/488 (Cl); found: molpeak (M+H)⁺: 486/488 (Cl); R_{f} value: 0.16 (silica gel, DCM/MeOH 9:1).

### EXAMPLE 6.4

### (S)-5-(4-chlorophenyl)-2-{6-[3-(3,5-dimethyllpiperidin-1-yl)prrolidin-1-yl]pyrid-3-ylethynyl}pyridine

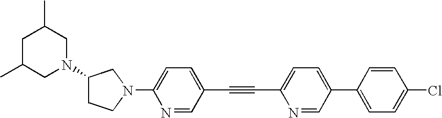

Analogously to 6.1e the product is obtained from 100 mg (0.18 mmol; 80% content) of 1-{5-[5-(4-chlorophenyl)pyridin-2-ylethynyl]pyridin-2-yl}pyrrolidin-3-yl (R)-methanesulfonate and 100 mg (0.88 mmol) of 3,5-dimethylpiperidine. Yield: 23 mg (27.7% of theoretical); C₂₉H₃₁ClN₄ (M=471.036); calc.: molpeak (M+H)⁺: 471/473 (Cl); found: molpeak (M+H)⁺: 471/473 (Cl); R_{f} value: 0.44 (silica gel, DCM/MeOH 9:1).

### EXAMPLE 6.5

### ((2S,3 ′S)-1′-{5-[5-(4-chlorophenyl)pyridin-2-ylethynyl]pyridin-2-yl}-[1,3′]-bipyrrolidinyl-2-yl)methanol

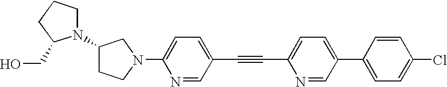

Analogously to 6.1e the product is obtained from 100 mg (0.18 mmol; 80% content) of 1-{5-[5-(4-chlorophenyl)pyridin-2-ylethynyl]pyridin-2-yl}pyrrolidin-3-yl (R)-methanesulfonate and 89 mg (0.88 mmol) of (S)-(+)-prolinole. Yield: 12 mg (14.9% of theoretical); C₂₇H₂₇ClN₄O (M=458.982); calc.: molpeak (M+H)⁺: 459/461 (Cl); found: molpeak (M+H)⁺: 459/461 (Cl); R_{f} value: 0.27 (silica gel, DCM/MeOH 9:1).

### EXAMPLE 6.6

### (S)-5-(4-chlorophenyl)-2-{6-[3-(4-methoxypiperidin-1-yl)pyrrolidin-1-yl]pyrid-3-ylethynyl}pyridine

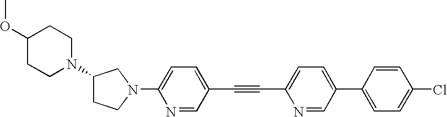

Analogously to 6.1e the product is obtained from 100 mg (0.18 mmol; 80% content) of 1-{5-[5-(4-chlorophenyl)pyridin-2-ylethynyl]pyridin-2-yl}pyrrolidin-3-yl (R)-methanesulfonate and 91 mg (0.88 mmol) of 4-methoxypiperidine. Yield: 9.0 mg (11.1% of theoretical); C₂₇H₂₇ClN₄O (M=458.982); calc.: molpeak (M+H)⁺: 459/461 (Cl); found: molpeak (M+H)⁺: 459/461 (Cl); R_{f} value: 0.33 (silica gel, DCM/MeOH 9:1).

### EXAMPLE 7.1

### (E)-5-(4-chlorophenyl)-2-{4-[3-(4-methylpiperidin-1-yl)propenyl]pyrid-3-ylethynyl}pyridine

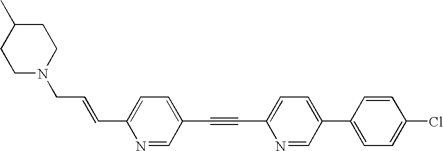 7.1a. 3-(5-bromopyridin-2-yl)prop-2-yn-1-ol

2.47 mL (42.2 mmol) of prop-2-yn-1-ol and 12.3 mL (88.2 mmol) of triethylamine are added to a solution of 10.0 g (42.2 mmol) of 2,5-dibromopyridine in 150 mL of THF. The reaction flask is evacuated and charged with argon. 88.4 mg (0.46 mmol) of CuI is added and stirred overnight at RT. The mixture is diluted with EtOAc and washed twice with 10% NH₃ solution and water. The organic phase is dried over magnesium sulfate and the solvent is eliminated in vacuo. Further purification is carried out by column chromatography on silica gel (PE/EtOAc 1:1). Yield: 6.70 g (75% of theoretical); C₈H₆BrNO (M=212.043); calc.: molpeak (M+H)⁺: 212/214 (Br); found: molpeak (M+H)⁺: 212/214 (Br); R_{f} value: 0.40 (silica gel, PE/EtOAc 1:1).

7.1b. (E)-3-(5-bromopyridin-2-yl)prop-2-en-1-ol

A solution of 1.00 g (4.72 mmol) of 3-(5-bromopyridin-2-yl)prop-2-yn-1-ol in 25 mL of THF is added dropwise at −5° C. to 4.72 mL (4.72 mmol, IM in THF) of a lithium aluminum hydride solution such that the internal temperature does not exceed 0° C. The mixture is stirred for 2 hours. Then 125 μL of water, 125 μL of 15% sodium hydroxide solution, and another 375 μL of water are added. The reaction mixture is filtered, dried over magnesium sulfate, and the solvent is eliminated in vacuo. Yield: 0.91 g (63% of theoretical); C₈H₈BrNO (M=214.059); calc.: molpeak (M+H)⁺: 214/216 (Br); found: molpeak (M+H)⁺: 214/216 (Br); retention time HPLC: 4.18 min (method B).

7.1c. (E)-3-(5-iodopyridin-2-yl)prop-2-en-1-ol

Prepared according to General Method II from (E)-3-(5-bromopyridin-2-yl)prop-2-en-1-ol (0.91 g, 4.25 mmol). Yield: 0.87 g (78% of theoretical); C₈H₈INO (M=261.060); calc.: molpeak (M+H)⁺: 262; found: molpeak (M+H)⁺: 262; retention time HPLC: 4.23 min (method B).

7.1d. (E)-3-{5-[5-(4-chlorophenyl)pyridin-2-ylethynyl]pyridin-2-yl}prop-2-en-1-ol

Prepared according to General Method I from (E)-3-(5-iodopyridin-2-yl)prop-2-en-1-ol (870 mg, 3.33 mmol) and 5-(4-chlorophenyl)-2-ethynylpyridine (712 mg, 3.33 mmol). Yield: 980 mg (85% of theoretical); C₂₁H₁₅ClN₂O (M=346.809); calc.: molpeak (M+H)⁺: 347/349 (Cl); found: molpeak (M+H)⁺: 347/349 (Cl); retention time HPLC: 5.57 min (method B).

7.1e. (E)-5-(4-chlorophenyl)-2-{4-[3-chloropropenyl]pyrid-3-ylethynyl}pyridine

A solution of 160 μL (1.35 mmol) of thionyl chloride (SOCl₂) in 5 mL of DCM is slowly added dropwise at −10° C. to a solution of 0.45 g (1.30 mmol) of (E)-3-{5-[5-(4-chlorophenyl)pyridin-2- ylethynyl]pyridin-2-yl}prop-2-en-1-ol in 20 mL of DCM. The reaction solution is stirred for 30 minutes at 0° C. and overnight at RT. After the addition of saturated sodium bicarbonate solution, the mixture is extracted with DCM. The organic phase is washed several times with water, dried over magnesium sulfate, and the solvent is eliminated in vacuo. Yield: 450 mg (95% of theoretical); C₂₁H₁₄Cl₂N₂ (M=365.255); calc.: molpeak (M+H)⁺: 365/367/369 (2Cl); found: molpeak (M+H)⁺: 365/367/369 (2Cl); retention time HPLC: 6.82 min (method B).

7.1 f. (E)-5-(4-chlorophenyl)-2-{4-[3-(4-methylpiperidin- 1-yl)propenyl]pyrid-3-ylethynyl}-pyridine

146 μL (1.23 mmol) of 4-methylpiperidine is added to a solution of 150 mg (0.41 mmol) of (E)-3-{5-[5-(4-chlorophenyl)pyridin-2-ylethynyl]pyridin-2-yl}allyl) chloride in 2 mL of DMF and the mixture is stirred overnight at 70° C. The purification is carried out by HPLC-MS. Yield: 42.0 mg (24% of theoretical); C₂₇H₂₆ClN₃ (M=427.968); calc.: molpeak (M+H)⁺: 428/430 (Cl); found: molpeak (M+H)⁺: 428/430 (Cl); retention time HPLC: 5.45 min (method A).

### EXAMPLE 7.2

### 1-((E)-3-{4-[5-(4-chlorophenyl)pyridin-2-ylethynyl]phenyl}allyl)-4-trifluoro-methylpiperidin-4-ol

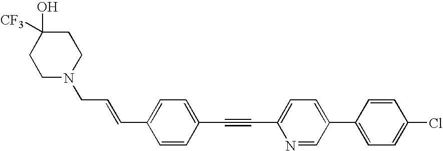 7.2a. (E)-3-(4-iodophenyl)prop-2-en-1-ol

Under a nitrogen atmosphere 12.0 g (80.0 mmol) of NaI and 0.85 mL (8.0 mmol) of N,N′-dimethylethylenediamine are added to a solution of 4.26 g (20.0 mmol) of (E)-3-(4-bromophenyl)prop-2-en-1-ol and 762 mg (4 mmol) of CuI in 20 mL of 1,4-dioxane and the reaction mixture is shaken for 17 hours at 110° C. The reaction mixture is cooled to RT, combined with 200 mL of EtOAc and 100 mL of semiconcentrated NH₃ solution, vigorously stirred, and the organic phase is separated off and dried over sodium sulfate. After the desiccant and solvent have been eliminated, the residue is reacted further without purification. Yield: 4.69 g (90% of theoretical); C₉H₉IO₂ (M=260.072); calc.: molpeak (M+H)⁺: 261; found: molpeak (M+H)⁺: 261; HPLC-MS: 7.9 min (method A).

7.2b. (E)-3-{4-[5-(4-chlorophenyl)pyridin-2-ylethynyl]phenyl}prop-2-en-1-ol

A solution of 3.12 g (12.0 mmol) of (E)-3-(4-iodophenyl)prop-2-en-1-ol, 3.33 g (15.0 mmol) of 5-(4-chlorophenyl)-2-ethynylpyridine, and 4.31 mL (24 mmol) of diisopropylamine in 120 mL of dry THF is evacuated three times and then gassed with argon. Then 45 mg (0.24 mmol) of CuI and 196 mg (0.24 mmol) of PdCI₂(dppf) are added. The reaction mixture is stirred for 18 hours at RT, the solvent is evaporated down in vacuo, and the residue is combined with 100 mL of DCM and 50 mL of semisaturated sodium bicarbonate solution and stirred vigorously. The precipitate is separated off, washed with water and a little DCM, suspended in DIPE, suction filtered again, and dried at 50° C. in the circulating air dryer until a constant weight is achieved. Yield: 4.23 g (quant. yield); C₂₂H₁₆ClNO (M=345.821); calc.: molpeak (M+H)⁺: 346/348 (Cl); found: molpeak (M+H)⁺: 346/348 (Cl); R_{f} value: 0.24 (silica gel, cyc/EtOAc 2: 1).

7.2c. 5-(4-chlorophenyl)-2-[4-((E)-3-chloropropenyl)phenylethynyl]pyridine

A solution of 2.56 mL (35.28 mmol) of thionyl chloride in 10 mL of DCM is slowly added dropwise to a solution of 6.1 g (17.64 mmol) of (E)-3-{4-[5-(4-chlorophenyl)pyridin-2-ylethynyl]phenyl}prop-2-en-1-ol in 80 mL of DCM which has been cooled to 0° C. and the reaction solution is stirred for a further 2 hours at 0° C. and 14 hours at RT. The mixture is again cooled to 0° C., 150 mL of semisaturated sodium bicarbonate solution is carefully added dropwise, and the organic phase is separated off and dried over sodium sulfate. After the desiccant and solvent have been eliminated, the residue is purified by chromatography (silica gel, cyc/EtOAc 4:1). Yield: 3.2 g (50% of theoretical); C₂₂H₁₅Cl₂N (M=364.267); calc.: molpeak (M+H)⁺: 364/366/368 (2Cl); found: molpeak (M+H)⁺: 364/366/368 (2Cl); R_{f} value: 0.60 (silica gel, cyc/EtOAc 2:1).

7.2d. 1-((E)-3-{4-[5-(4-chlorophenyl)pyridin-2-ylethynyl]phenyl}allyl)-4-trifluoromethyl-piperidin-4-ol

65.0 mg (0.38 mmol) of 4-trifluoromethylpiperidin-4-ol and 0.13 mL (0.77 mmol) of ethyldiisopropylamine are added to a solution of 70.0 mg (0.19 mmol) of 5-(4-chlorophenyl)-2-[4-((E)-3-chloropropenyl)phenylethynyl]pyridine in 1.7 mL of DMF and shaken for 19 hours at 60° C. The reaction mixture is filtered through an injection filter and purified by HPLC-MS. The residue is diluted with 20 mL of EtOAc and 10 mL of saturated sodium bicarbonate solution. The organic phase is dried over sodium sulfate and the solvent is eliminated in vacuo. It is then stirred with DIPE. Yield: 40.4 mg (42.0% of theoretical); C₂₈H₂₄ClF₃N₂O (M=496.951); calc.: molpeak (M+H)⁺: 497/499 (Cl); found: molpeak (M+H)⁺: 497/499 (Cl); retention time HPLC: 5.5 min (method B).

### EXAMPLE 7.3

### ((E)-3-{4-[5-(4-chlorophenyl)pyridin-2-ylethynyl]phenyl}allyl)cyclopropyl-methylpropylamine

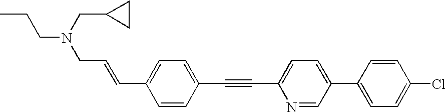

0.11 mL (0.77 mmol) of cyclopropylmethylpropylamine and 0.13 mL (0.77 mmol) of ethyldiisopropylamine are added to a solution of 70.0 mg (0.19 mmol) of 5-(4-chlorophenyl)-2-[4-((E)-3-chloropropenyl)phenylethynyl]pyridine in 1.7 mL of DMF and the mixture is shaken for 3.5 hours at 60° C. The reaction mixture is purified by HPLC-MS. Yield: 45.3 mg (53.0% of theoretical); C₂₉H₂₉ClN₂ (M=441.007); calc.: molpeak (M+H)⁺: 441/443 (Cl); found: molpeak (M+H)⁺: 441/443 (Cl); retention time HPLC: 5.6 min (method B).

### EXAMPLE 7.4

### 2-((E)-3-{4-[5-(4-chlorophenyl)pyridin-2-ylethynyl]phenyl}allylamino)-2-methylpropane-1.3-diol

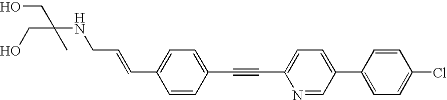

80.7 mg (0.77 mmol) of 2-amino-2-methyl-1,3-propanediol and 0.13 mL (0.77 mmol) of ethyldiisopropylamine are added to a solution of 70.0 mg (0.19 mmol) of 5-(4-chlorophenyl)-2-[4-((E)-3-chloropropenyl)phenylethynyl]pyridine in 1.7 mL of DMF and shaken for 17 hours at 60° C. The reaction mixture is purified by HPLC-MS. Yield: 38.2 mg (46.0% of theoretical); C₂₆H₂₅ClN₂O₂ (M=432.942); calc.: molpeak (M+H)⁺: 433/435 (Cl); found: molpeak (M+H)⁺: 433/435 (Cl); retention time HPLC: 4.9 min (method B).

### EXAMPLE 7.5

### ((E)-3-{4-[5-(4-chlorophenyl)pyridin-2-ylethynyl]phenyl}allyl)cyclopentylamine

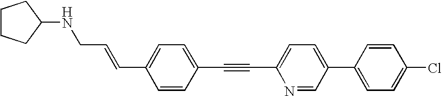

0.23 mL (2.30 mmol) of cyclopentylamine and 0.39 mL (2.30 mmol) of ethyidiisopropylamine are added to a solution of 210 mg (0.58 mmol) of 5-(4-chlorophenyl)-2-[4-((E)-3-chloropropenyl)phenylethynyl]pyridine in 5.1 mL of DMF and shaken for 17 hours at 60° C. The reaction mixture is purified by HPLC-MS. Yield: 107 mg (45.0% of theoretical); C₂₇H₂₅ClN₂ (M=412.954); calc.: molpeak (M+H)⁺: 413/415 (Cl); found: molpeak (M+H)⁺: 413/415 (Cl); retention time HPLC: 5.6 min (method B).

### EXAMPLE 7.6

### 1-((E)-3-{4-[5-(4-chlorophenyl)pyridin-2-ylethynyl]phenyl}allyl)-4-methyl-piperidin-4-ol

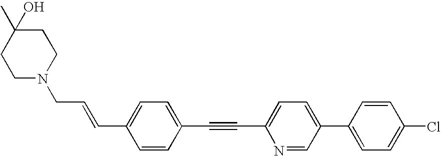

44.2 mg (0.38 mmol) of 4-methylpiperidin-4-ol and 0.13 mL (0.77 mmol) of ethyldllsopropylamine are added to a solution of 70.0 mg (0.19 mmol) of 5-(4-chlorophenyl)-2-[4-((E)-3-chloropropenyl)phenylethynyl]pyridine in 1.7 ML of DMF and shaken for 17 hours at 60° C. The reaction mixture is purified by HPLC-MS. Yield: 58.0 mg (68.0% of theoretical); C₂₈H₂₇ClN₂O (M=442.980); calc.: molpeak (M+H)⁺: 443/445 (Cl); found: molpeak (M+H)⁺: 443/445 (Cl); retention time HPLC: 5.2 min (method B).

### EXAMPLE 7.7

### 2-((E)-3-{4-[5-(4-chlorophenyl)pyridin-2-ylethynyl]phenyl}allylamino)propane- 1,3-diol

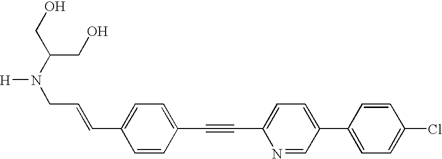

70.0 mg (0.77 mmol) of 2-amino-1,3-propanediol and 0.13 mL (0.77 mmol) of ethyldiisopropylamine are added to a solution of 70.0 mg (0.19 mmol) of 5-(4-chlorophenyl)-2-[4-((E)-3-chloropropenyl)phenylethynyl]pyridine in 1.7 mL of DMF and shaken for 3.5 hours at 60° C. The reaction mixture is purified by HPLC-MS. Yield: 37.6 mg (47.0% of theoretical); C₂₅H₂₃ClN₂O₂ (M=418.915); calc.: molpeak (M+H)⁺: 419/421 (Cl); found: molpeak (M+H)⁺: 419/421 (Cl); retention time HPLC: 4.9 min (method B).

### EXAMPLE 7.8

### 3-[((E)-3-{4-[5-(4-chlorophenyl)pyridin-2-ylethynyl]phenyl}allyl)cyclopentyl-amino]propan-1-ol

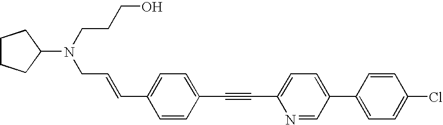

61.0 μL (0.68 mmol) of 3-bromo-1-propanol and 0.12 mL (0.68 mmol) of ethyldiisopropylamine are added to a solution of 70.0 mg (0.17 mmol) of ((E)-3-{4-[5-(4-chlorophenyl)pyridin-2-ylethynyl]phenyl}allyl)cyclopentylamine in 1.7 mL of DMF and shaken for 17 hours at 40° C. Another 61.0 μL (0.68 mmol) of 3-bromo-1-propanol is added and the mixture is shaken for 23 hours at 40° C. The solvent is eliminated in vacuo and the residue is taken up with 20 mL of EtOAc and 10 mL of semisaturated sodium bicarbonate solution. The organic phase is dried over sodium sulfate and the solvent is eliminated in vacuo. The residue is then stirred with TBME. Yield: 9.20 mg (11.5% of theoretical); C₃₀H₃₁ClN₂O (M=471.033); calc.: molpeak (M+H)⁺: 471/473 (Cl); found: molpeak (M+H)⁺: 471/473 (Cl); R_{f} value: 0.30 (silica gel, 366 nm, DCM/MeOH/NH₃ 9:1:0.1).

### EXAMPLE 7.9

### 8-((E)-3-{4-[5-(4-chlorophenyl)pyridin-2-ylethynyl]phenyl}allyl)-3-methyl-8-azabicyclo[3.2]octan-3-ol

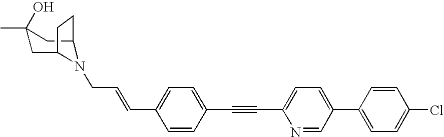

46.6 mg (0.33 mmol) of 3-methyl-8-azabicyclo[3.2.1]octan-3-ol and 0.11 mL (0.66 mmol) of ethyldiisopropylamine are added to a solution of 60.0 mg (0.17 mmol) of 5-(4-chlorophenyl)-2-[4-((E)-3-chloropropenyl)phenylethynyl]pyridine in 1.7 mL of DMF and the reaction mixture is shaken for 16 hours at 60° C. The reaction mixture is evaporated down in vacuo, and the residue is taken up in 20 mL of EtOAc and 10 mL of 5% sodium bicarbonate solution, briefly heated to 80° C., and the organic phase is separated off and dried over sodium sulfate. After the desiccant and solvent have been eliminated, the residue is stirred with 5 mL isopropanol and suction filtered. Yield: 48.4 mg (63% of theoretical); C₃₀H₂₉ClN₂O (M=469.017); calc.: molpeak (M+H)⁺: 469/471 (Cl); found: molpeak (M+H)⁺: 469/471 (Cl); retention time HPLC: 4.9 min (method B).

### EXAMPLE 7.10

### 8-((E)-3-{4-[5-(4-chlorophenyl)pyridin-2-ylethynyl]phenyl}allyl)-3-ethyl-8-azabicyclo[3.2.1]octan-3-ol

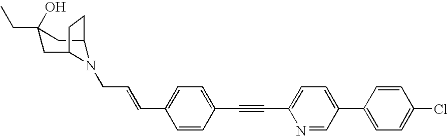

Analogously to Example 7.9 the product is obtained from 60.0 mg (0.17 mmol) of 5-(4-chlorophenyl)-2-[4-((E)-3-chloropropenyl)phenylethynyl]pyridine and 51.2 mg (0.33 mmol) of 3-ethyl-8-azabicyclo-[3.2.1]octan-3-ol. Yield: 32.9 mg (41% of theoretical); C₃₁H₃₁ClN₂O (M=483.043); calc.: molpeak (M+H)⁺: 483/485 (Cl); found: molpeak (M+H)⁺: 483/485 (Cl); retention time HPLC: 5.0 min (method B).

### EXAMPLE 7.11

### 1-((E)-3-{4-[5-(4-chlorophenyl)pyridin-2-ylethynyl]phenyl}allyl)-4-ethylpiperidin-4-ol

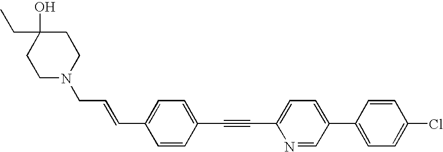

42.6 mg (0.33 mmol) of 4-ethylpiperidin-4-ol and 0.08 mL (0.44 mmol) of ethyldiisopropylamine are added to a solution of 40.0 mg (0.11 mmol) of 5-(4-chlorophenyl)-2-[4-((E)-3-chloropropenyl)phenylethynyl]pyridine in 1.7 mL of DMF and the reaction mixture is shaken for 5 hours at 60° C. After cooling, the reaction mixture is purified by HPLC without working up. The fractions containing the product are combined with 20 mL of EtOAc and 10 mL of 5% sodium bicarbonate solution and stirred. The phases are separated and the organic phase is dried over sodium sulfate. After the desiccant and solvent have been eliminated, the residue is triturated with a little DIPE, suction filtered, and dried. Yield: 19.9 mg (40% of theoretical); C₂₉H₂₉ClN₂O (M=457.006); calc.: molpeak (M+H)⁺: 457/459 (Cl); found: molpeak (M+H)⁺: 457/459 (Cl); retention time HPLC: 4.9 min (method B).

### EXAMPLE 7.12

### 4-methyl-1-((E)-3-{4-[5-(4-methylcyclohex-1-enyl)pyridin-2-ylethynyl]phenyl}-allyl)piperidin-4-ol

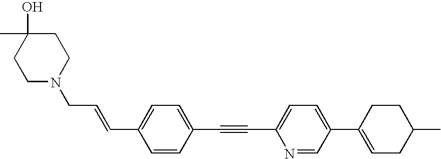 7.12a. 1-{6-[(tert-butyldimethylsilanyl)ethynyl]pyridin-3-yl}-4-methylcyclohexanol

9.5 mL (16.19 mmol) of n-BuLi (1.6 M in THF) is slowly added under argon at −70° C. to a solution of 4.50 g (15.19 mmol) of 5-bromo-2-[(tert-butyldimethylsilanyl)ethynyl]pyridine in 50 mL of diethyl ether and 60 mL of THF and, after the addition has ended, the mixture is stirred for another 2 minutes, 1.86 mL (15.19 mmol) of 4-methylcyclohexanone is added, and the mixture is slowly heated to RT. 150 mL of saturated ammonium chloride solution is added and the aqueous phase is exhaustively extracted with EtOAc. The combined organic extracts are washed with semisaturated sodium bicarbonate solution, dried over magnesium sulfate, and evaporated down in vacuo. The residue is purified by chromatography (silica gel, PE/EtOAc 4:1). 2 fractions are isolated:

fraction 1: cis-1-{6-[(tert-butyldimethylsilanyl)ethynyl]pyridin-3-yl}-4-methylcyclohexanol Yield: 1.40 g (28% of theoretical); C₂₀H₃₁NOSi (M=329.552); calc.: molpeak (M+H)⁺: 330; found: molpeak (M+H)⁺: 330; R_{f} value: 0.40 (silica gel, PE/EtOAc 4:1).

fraction 2: trans-1-{6-[(tert-butyldimethylsilanyl)ethynyl]pyridin-3-yl}-4-methylcyclohexanol Yield: 1.00 g (20% of theoretical); C₂₀H₃₁,NOSi (M=329.552); calc.: molpeak (M+H)⁺: 330; found: molpeak (M+H)⁺: 330; R_{f} value: 0.30 (silica gel, PE/EtOAc 4:1)

7.12b. 2-[(tert-butyldimethylsilanyl)ethynyl]-5-(4-methylcyclohex-1-enyl)pyridine

1.22 mL (15.78 mmol) of methanesulfonic acid chloride is slowly added dropwise to a solution cooled to 0° C. of 1.3 g (3.95 mmol) of cis-1-{6-[(tert-butyldimethylsilanyl)ethynyl]pyridin-3-yl}-4-methylcyclohexanol and 2.2 mL (15.78 mmol) of triethylamine in 30 mL of DCM. After the addition has ended, the cooling bath is removed and the reaction mixture is stirred for 2 hours at RT. To complete the reaction, another 2.2 mL of triethylamine and 1.22 mL of methanesulfonic acid chloride are added, the mixture is stirred overnight at RT, and again combined with 1 mL of triethylamine. Water is added to the reaction solution, and the organic phase is separated off, washed with water, and dried over magnesium sulfate. After the desiccant and solvent have been eliminated, the residue is purified by chromatography (silica gel, gradient PE to PE/EtOAc 9:1). Yield: 0.95 g (20% of theoretical); C₂₀H₂₉NSi (M=311.536); calc.: molpeak(M+H)⁺: 312; found: molpeak(M+H)⁺: 312

7.12c. 2-ethynyl-5-(4-methylcyclohex- 1-enyl)pyridine

877 mg (3.35 mmol) of TBAF is added to a solution of 950 mg (3.05 mmol) of 2-[(tert-butyldimethylsilanyl)ethynyl]-5-(4-methylcyclohex-1-enyl)pyridine in 20 mL of DCM and the reaction solution is stirred for 30 minutes at RT. The mixture is combined with water, and the organic phase is separated off, washed three times with water, and dried over magnesium sulfate. After the desiccant and solvent have been eliminated, the residue is purified by chromatography (Alox, PE/EtOAc 9:1). Yield: 400 mg (66% of theoretical); C₁₄H₁₅N (M=197.276); calc.: molpeak (M+H)⁺: 198; found: molpeak (M+H)⁺: 198; retention time HPLC: 5.9 min (method E).

7.12d. 1-bromo-4-((E)-3-chloropropenyl)benzene

1.67 mL (20.65 mmol) of pyridine and one drop of DMF are added to a solution cooled to 0° C. of 4.40 g (20.65 mmol) of (E)-3-(4-bromophenyl)prop-2-en-1-ol in 50 mL of DCM and then a solution of 1.51 mL (20.65 mmol) of thionyl chloride in 10 mL of DCM is slowly added dropwise. The reaction mixture is stirred for 1 hour at 0° C. and for 3 hours at RT. It is carefully combined with water, and the organic phase is separated off, washed with water, and dried over magnesium sulfate. After the desiccant and solvent have been eliminated, the residue is purified by chromatography (silica gel, PE/DCM 4:1). Yield: 1.9 g (40% of theoretical); C₉H₈BrCl (M=231.517); calc.: molpeak (M+H)⁺: 230/232/234 (BrCl); found: molpeak (M+H)⁺: 230/232/234 (BrCl).

7.12e. 1-[(E)-3-(4-bromophenyl)allyl]-4-methylpiperidin-4-ol

709 mg (6.16 mmol) of 4-methylpiperidin-4-ol is added to a solution of 950 mg (4.10 mmol) of 1-bromo-4-((E)-3-chloropropenyl)benzene and 1.14 mL (8.21 mmol) of triethylamine in 5 mL of DMF and the reaction mixture is stirred overnight at RT. It is evaporated down in vacuo, the residue is taken up in water and extracted exhaustively with EtOAc, and the combined organic phases are washed with water and dried over magnesium sulfate. After the desiccant and solvent have been eliminated, the residue is evaporated down in vacuo and reacted further without purification. Yield: 600 mg (47% of theoretical); C₁₅H₂₀BrNO (M=310.229); calc.: molpeak (M+H)⁺: 310/312 (Br); found: molpeak (M+H)^{+:} 310/312 (Br).

7.12f 1-[(E)-3-(4-iodophenyl)allyl]4-methylpiperidin-4-ol

Prepared according to General Method II from 600 mg (1.93 mmol) of 1-[(E)-3-(4-bromophenyl)allyl]-4-methylpiperidin-4-ol. Yield: 700 mg (100% of theoretical); C₁₅H₂₀INO (M=357.230); calc.: molpeak (M+H)⁺: 358; found: molpeak (M+H)⁺: 358; retention time HPLC: 3.5 min (method B).

7.12g. 4-methyl-1-((E)-3-{4-[5-(4-methylcyclohex-1-enyl)pyridin-2-ylethynyl]phenyl}allyl)-piperidin-4-ol

Prepared according to General Method I from 120 mg (0.34 mmol) of 1-[(E)-3-(4-iodophenyl)allyl]-4-methylpiperidin-4-ol and 80 mg (0.40 mmol) of 2-ethynyl-5-(4-methylcyclohex-1-enyl)pyridine (with Pd(dppf)Cl₂ as catalyst, triethylamine as base, and THF as solvent). Yield: 30 mg (21% of theoretical); C₂₉H₃₄N₂O (M=426.593); calc.: molpeak (M+H)⁺: 427; found: molpeak (M+H)+: 427; retention time HPLC: 5.6 min (method D).

### EXAMPLE 7.13

### 5-(4-methylcyclohex-1-enyl)-2-{4-[(E)-3-(4-methylpiperidin-1-yl)propenyl]-phenylethynyl}pyridine

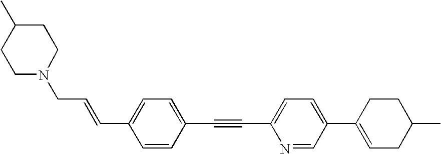 7.13a. 1-[(E)-3-(4-bromophenyl)allyl]-4-methylpiperidine

1.94 mL (16.41 mmol) of 4-methylpiperidine is added to a solution of 950 mg (4.10 mmol) of 1-bromo-4-((E)-3-chloropropenyl)benzene in 5 mL of DMF and the reaction solution is stirred overnight at RT. It is evaporated down in vacuo, the residue is taken up in water and extracted exhaustively with EtOAc, and the combined organic phases are washed with water and dried over magnesium sulfate. After the desiccant and solvent have been eliminated, the residue is evaporated down in vacuo and reacted further without purification. Yield: 900 mg (75% of theoretical); C₁₅H₂₀BrN (M=294.230); calc.: molpeak (M+H)⁺: 294/296 (Br); found: molpeak (M+H)⁺: 294/296 (Br).

7.13b. 1-[(E)-3-(4-iodophenyl)allyl]-4-methylpiperidine

Prepared according to General Method II from 900 mg (3.06 mmol) of 1-[(E)-3-(4-bromophenyl)allyl]4-methylpiperidine. Yield: 1.0 g (96% of theoretical); C₁₅H₂₀IN (M=341.231); calc.: molpeak (M+H)⁺: 342; found: molpeak (M+H)⁺: 342; retention time HPLC: 4.0 min (method B).

7.13c. 5-(4-methylcyclohex-1-enyl)-2-{4-[(E)-3-(4-methylpiperidin-1-yl)propenyl]phenylethynyl}pyridine

Prepared according to General Method I from 120 mg (0.34 mmol) of 1-[(E)-3-(4-iodophenyl)allyl]-4-methylpiperidine and 83 mg (0.42 mmol) of 2-ethynyl-5-(4-methylcyclohex-1-enyl)pyridine (with Pd(dppf)Cl₂ as catalyst, triethylamine as base, and THF as solvent). Yield: 30 mg (21% of theoretical); C₂₉H₃₄N₂ (M=410.594); calc.: molpeak (M+H)⁺: 411; found: molpeak (M+H)^{+:} 411; retention time HPLC: 6.0 min (method D).

### EXAMPLE 7.14

### 1-((E)-3-{4-[5-(4-chlorophenyl)-3-fluoropyridin-2-ylethynyl]phenyl}allyl)-4-methylpiperidin-4-ol

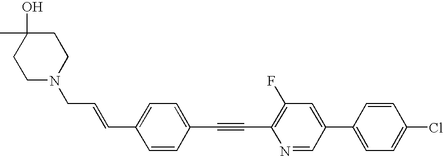 7.14a. 2,5-dibromo-3-fluoropyridine

A solution of 1.78 g (25.80 mmol) of sodium nitrite in 3.5 mL of water is added dropwise at −5° C. to a solution of 6.50 g (25.80 mmol) of 2,5-dibromopyridin-3-ylamine and 15 mL of concentrated HCl (180.62 mmol) in 15 mL of water and the mixture is stirred for 30 minutes. At 0° C., 11.41 mL (77.41 mmol) of hexafluorophosphoric acid (60% in water) is added and the mixture is stirred for 1 hour at 0° C. The diazonium salt formed is filtered off, washed with cold water, isopropanol, and diethyl ether, and dried in vacuo in the desiccator. PE (boiling range 100° C.-140° C.) is heated to 90° C., the diazonium salt is added batchwise and the mixture is stirred until no further development of gas can be detected. The reaction mixture is cooled to RT, made alkaline with saturated sodium carbonate solution, and the aqueous phase is exhaustively extracted with TBME. The combined organic phases are washed with saturated sodium carbonate solution and water and dried over magnesium sulfate. After the desiccant and solvent have been eliminated, the residue is dissolved in DCM, filtered through silica gel, and the filtrate is evaporated down in vacuo. Yield: 3.30 (51% of theoretical); C₅H₂Br₂FN (M=254.883); calc.: molpeak (M+H)⁺: 253/255/257 (2 Br); found: molpeak (M+H)⁺: 253/255/257 (2 Br); R_{f} value: 0.63 (silica gel, PE/EtOAc 9:1).

7.14b. 5-bromo-2-[(tert-butyldimethylsilanyl)ethynyl]-3-fluoropyridine

Under an argon atmosphere 2.62 mL (13.81 mmol) of tert-butylethynyldimethylsilane is added at 15° C. to a solution of 3.20 g (12.56 mmol) of 2,5-dibromo-3-fluoropyridine, 5.22 mL of triethylamine (37.67 mmol), 59.8 mg (0.31 mmol) of CuI, and 220.3 mg (0.31 mmol) of bis(triphenylphosphane)palladium (II) chloride in 30 mL of dry THF and the mixture is stirred for 2 hours at RT. 1 mL of tert-butylethynyldimethylsilane is added and again the mixture is stirred for 1 hour at RT. The reaction mixture is evaporated down in vacuo and the residue is taken up in EtOAc. The organic phase is washed with semisaturated sodium carbonate solution, 5% NH₃ solution, and water and dried over magnesium sulfate. After the desiccant and solvent have been eliminated, the residue is purified by chromatography (silica gel, PE/DCM 9:1). Yield: 1.62 g (41% of theoretical); C₁₃H₁₇BrFNSi (M=314.269); calc.: molpeak (M+H)⁺: 314/316 (Br); found: molpeak (M+H)⁺: 314/316 (Br); HPLC-MS: 7.9 min (method B).

7.14c. 2-[(tert-butyldimethylsilanyl)ethynyl]-5-(4-chlorophenyl)-3-fluoropyridine

10 mL of MeOH, 10 mL of 2N aqueous sodium carbonate solution, and 94 mg (0.13 mmol) of Pd(dppf)Cl₂ are added to a solution of 1.61 g (5.14 mmol) of 5-bromo-2-[(tert-butyldimethylsilanyl)ethynyl]-3-fluoropyridine and 0.90 g (5.65 mmol) of 4-chlorophenylboric acid in 30 mL of 1,4-dioxane and the mixture is refluxed for 15 minutes. The reaction mixture is evaporated down in vacuo and diluted with EtOAc. The organic phase is washed with water and semisaturated sodium carbonate solution and dried over sodium sulfate. After the desiccant and solvent have been eliminated, the residue is purified by chromatography (silica gel, PE/DCM 1:1). Yield: 1.25 g (70% of theoretical); C₁₉H₂₁ClFNSi (M=345.913); calc.: molpeak (M+H)⁺: 346/348 (Cl); found: molpeak (M+H)⁺: 346/348 (Cl); HPLC-MS: 8.9 min (method B).

7.14d. 5-(4-chlorophenyl)-2-ethynyl-3-fluoropyridine

1.14 g (3.61 mmol) of TBAF is added at RT to a solution of 1.25 g (3.61 mmol) of 2-[(tert-butyldimethylsilanyl)ethynyl]-5-(4-chlorophenyl)-3-fluoropyridine in 30 mL of DCM and the mixture is stirred for 2 hours at RT. The organic phase is washed with water and dried over sodium sulfate. After the desiccant and solvent have been eliminated, the residue is stirred with PE, and the precipitate is filtered off, washed with PE, and dried in the air. Yield: 0.72 g (86% of theoretical); C₁₃H₇ClFN (M=231.653); calc.: molpeak (M+H)⁺: 232/234 (Cl); found: molpeak (M+H)⁺: 232/234 (Cl); HPLC-MS: 5.8 min (method B).

7.14e. 1-((E)-3-{4-[5-(4-chlorophenyl)-3-fluoropyridin-2-ylethynyl]phenyl}allyl)-4-methylpiperidin-4-ol

Prepared according to General Method I from 200 mg (0.56 mmol) of 1-[(E)-3-(4-iodophenyl)allyl]-4-methylpiperidin-4-ol and 130 mg (0.56 mmol) of 5-(4-chlorophenyl)-2-ethynyl-3-fluoropyridine (with Pd(dppf)Cl₂ as catalyst, triethylamine as base, and THF as solvent). Yield: 55 mg (21% of theoretical); C₂₈H₂₆ClFN₂O (M=460.970); calc.: molpeak (M+H)⁺: 461/463 (Cl); found: molpeak (M+H)⁺: 461/463 (Cl); retention time HPLC: 5.6 min (method D).

### EXAMPLE 7.15

### 5-(4-chlorophenyl)-3-fluoro-2-{4-[(E)-3-(4-methylpiperidin-1-yl)propenyl]-phenylethynyl}pyridine

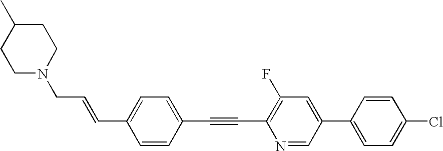

Prepared according to General Method I from 200 mg (0.59 mmol) of 1-[(E)-3-(4-iodophenyl)allyl]-4-methylpiperidine and 136 mg (0.59 mmol) of 5-(4-chlorophenyl)-2-ethynyl-3-fluoropyridine (with Pd(dppf)Cl₂ as catalyst, triethylamnine as base, and THF as solvent). Yield: 40 mg (15% of theoretical); C₂₈H₂₆ClFN₂ (M=444.971); calc.: molpeak (M+H)⁺: 445/447 (Cl); found: molpeak (M+H)⁺: 445/447 (Cl); retention time HPLC: 6.0 min (method D).

### EXAMPLE 8

### (E)-5-(4-chlorophenyl)-2-{4-[2-methyl-3-(4-methylpiperidin-1-yl)propenyl]phenyl-ethynyl}pyridine

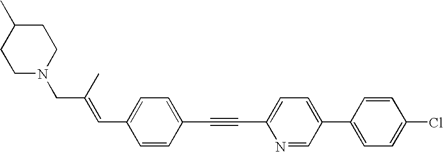 8a. 2-(4-bromophenylethynyl)-5-(4-chlorophenyl)pyridine

Prepared according to General Method I from 4-bromoiodobenzene (566 mg, 2.00 mmol) and 5-(4-chlorophenyl)-2-ethynylpyridine (460 mg, 2.00 mmol). Yield: 600 mg (81.4% of theoretical); C₁₉H₁₁,BrClN (M=368.654); calc.: molpeak (M+H)⁺: 368/370/372 (BrCl); found: molpeak (M+H)⁺: 368/370/372 (BrCl) R_{f} value: 0.78 (silica gel, PE/DCM 1:1).

8b. 5-(4-chlorophenyl)-2-(4-iodophenylethynyl)pyridine

Prepared according to General Method II from 2-(4-bromophenylethynyl)-5-(4-chlorophenyl)pyridine (600 mg, 1.63 mmol). Yield: 500 mg (73.9% of theoretical); C₁₉H₁₁ClIN (M=415.655); calc.: molpeak (M)⁺: 416/418 (Cl); found: molpeak (M)⁺: 416/418 (Cl); retention time HPLC: 7.76 min (method B).

8c. (E)-3-{4-[5-(4-chlorophenyl)pyridin-2-ylethynyl]phenyl}-2-methylprop-2-en-1-ol

195 mg (2.70 mmol) of 2-methyl-2-propen-1-ol, 200 mg (1.20 mmol) of silver acetate, 13 mg (0.06 mmol) of palladium (II) acetate, and 31 mg (0.12 mmol) of triphenylphosphane are added successively to a solution of 500 mg (1.20 mmol) of 5-(4-chlorophenyl)-2-(4-iodophenylethynyl)pyridine in 2.0 mL of DMF in an argon atmosphere. The reaction mixture is shaken for 2 days at 75° C. A further 89 mg (0.55 mmol) of 2-methyl-2-propen-1-ol is added and the reaction mixture is stirred for a further 4 days at 75° C. After cooling, the reaction solution is diluted with 70 mL of DCM and 30 mL of water. After filtration, the organic phase is dried over magnesium sulfate and the solvent is eliminated in vacuo. Further purification is carried out by column chromatography on silica gel (PE towards PE/EtOAc 7:3). Yield: 60 mg (13.9% of theoretical); C₂₃H₂₀ClNO (M=359.85); calc.: molpeak (M+H)⁺: 360/362 (Cl); found: molpeak (M+H)⁺: 360/362 (Cl); retention time HPLC: 6.30 min (method B).

8d. (E)-5-(4-chlorophenyl)-2-{4-[2-methyl-3-(4-methylpiperidin-1-yl)propenyl]phenyl-ethynyl}pyridine

15.0 μL (0.19 mmol) of methanesulfonic acid chloride and 30 μL (0.22 mmol) of triethylamine are added to a solution of 60.0 mg (0.17 mmol) of (E)-3-{4-[5-(4-chlorophenyl)pyridin-2-ylethynyl]phenyl}-2-methylprop-2-en-1-ol in 5.0 mL of DCM. The reaction is stirred for 4 hours at RT and a further 15.0 μL (0.19 mmol) of methanesulfonic acid chloride and 30 μL (0.22 mmol) of triethylamine are added. The reaction solution is stirred for 2 hours at RT and then 0.50 mL (4.23 mmol) of 4-methylpiperidine is added. The reaction mixture is stirred for 2 hours at RT and then diluted with 30 mL of DCM. The organic phase is washed three times with water, dried over magnesium sulfate, and the solvent is eliminated in vacuo. Further purification is carried out by column chromatography on silica gel (DCM towards DCM/MeOH NH₃ 9:1:0.1). Yield: 10 mg (13.6% of theoretical); C₂₉H₂₉ClN₂ (M=440.988); calc.: molpeak (M+H)⁺: 441/443 (Cl); found: molpeak (M+H)⁺: 441/443 (Cl); R_{f} value: 0.15 (silica gel, DCM/MeOH/NH₃ 95:5:0.5).

### EXAMPLE 8.1

### 1-((E)-3-{4-[5-(4-chlorophenyl)pyridin-2-ylethynyl]phenyl}-2-methylallyl)-4-methylpiperidin-4-ol

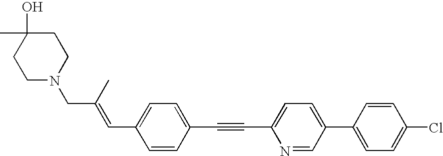 8.1a. 2-[4-((E)-3-chloro-2-methylpropenyl)phenylethynyl]-5-(4-chlorophenyl)pyridine

0.27 mL (3.36 mmol) of pyridine is added to a solution cooled to 0° C. of 1.10 g (3.06 mmol) of (E)-3-{4-[5-(4-chlorophenyl)pyridin-2-ylethynyl]phenyl}-2-methylprop-2-en-1-ol (Example 8c) in 100 mL of DCM and then 0.25 mL (3.36 mmol) of thionyl chloride is slowly added dropwise. The reaction solution is brought to RT and stirred for 1 hour. The mixture is combined with ice water, and the organic phase is separated off, washed several times with water, and dried over magnesium sulfate. After the desiccant and solvent have been eliminated, the residue is purified by chromatography (silica gel, gradient PE to PE/DCM 1:4). Yield: 160 mg (14% of theoretical); C₂₃H₁₇Cl₂N (M=378.293); calc.: molpeak (M+H)⁺: 378/380/382 (2 Cl); found: molpeak (M+H)⁺: 378/380/382 (2 Cl); retention time HPLC: 7.9 min (method F).

8.1b. 1-((E)-3-{4-[5-(4-chlorophenyl)pyridin-2-ylethynyl]phenyl}-2-methylallyl)-4-methylpiperidin-4-ol

22 mg (0.19 mmol) of 4-methylpiperidin-4-ol is added to a solution of 40 mg (0.11 mmol) of 2-[4-((E)-3-chloro-2-methylpropenyl)phenylethynyl]-5-(4-chlorophenyl)pyridine in 1 mL of DMF and the reaction mixture is stirred for 1 hour at 60° C. The reaction solution is purified by HPLC without any further working up. The fractions containing the product are combined and lyophilized. Yield: 20 mg (39% of theoretical); C₂₉H₂₉ClN₂O (M=457.006); calc.: molpeak (M+H)⁺: 457/459 (Cl); found: molpeak (M+H)⁺: 457/459 (Cl); retention time HPLC: 7.4 min (method A).

The following Examples are prepared analogously, in each case starting from 40 mg of 2-[4-((E)-3-chloro-2-methylpropenyl)phenylethynyl]-5-(4-chlorophenyl)pyridine.

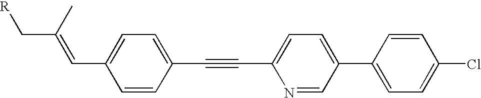

| | | | | | HPLC |
|---|---|---|---|---|---|
| | | | | Mass | retention time |
| Example | R | Yield (%) | Empirical formula | spectrum | (method) |
| 8.2 | 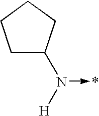 | 42 | C₂₈H₂₇ClN₂ | 427/429 [M + H]⁺ | 8.0 min (A) |
| 8.3 | 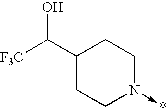 | 34 | C₃₀H₂₈ClF₃N₂O | 525/527 [M + H]⁺ | 8.1 min (A) |

### EXAMPLE 8.4

### 5-(4-chlorolphenyl)-3-fluoro-2-{4-[(E)-2-methyl-3-(4-methylpiperidin-1-yl)propenyl]phenylethynyl}pyridine

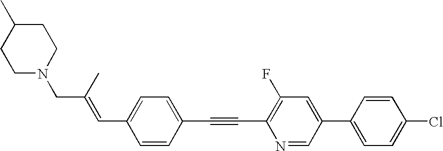 8.4a. (E)-3-(4-iodophenyl)-2-methylprop-2-en-1-ol

Prepared according to General Method II from 1.0 g (4.40 mmol) of (E)-3-(4-bromophenyl)-2-methylprop-2-en-1-ol. Yield: 1.1 g (91% of theoretical); C₁₀H₁₁IO (M=274.098); calc.: molpeak (M)⁺: 274; found: molpeak (M)⁺: 274; retention time HPLC: 5.5 min (method B).

8.4b. 1-((E)-3-chloro-2-methylpropenyl)-4-iodobenzene

Prepared analogously to Example 7.12d from 1.10 g (4.01 mmol) of (E)-3-(4-iodophenyl)-2-methylprop-2-en-1-ol and 0.35 mL (4.82 mmol) of thionyl chloride, while in order to complete the reaction the solution is stirred overnight at RT. Yield: 1.1 g (94% of theoretical); C₁₀H₁₀ClI (M=292.544); calc.: molpeak (M)⁺: 292/294 (Cl); found: molpeak (M)^{+:} 292/294 (Cl).

8.4c. 1-[(E)-3-(4-iodophenyl)-2-methylallyl]4-methylpiperidine

0.89 mL (7.52 mmol) of 4-methylpiperidine is added to a solution of 550 mg (1.88 mmol) of 1-((E)-3-chloro-2-methylpropenyl)4-iodobenzene in 5 mL of DMF and the reaction mixture is stirred overnight at RT. Water is added to the reaction solution, it is exhaustively extracted with EtOAc, and the combined organic phases are dried over magnesium sulfate. After the desiccant and solvent have been eliminated, the residue is purified by chromatography (silica gel, gradient PE to PE/EtOAc 4:1). Yield: 140 mg (21% of theoretical); C₁₆H₂₂IN (M=355.257); calc.: molpeak (M+H)⁺: 356; found: molpeak (M+H)⁺: 356; retention time HPLC: 5.8 min (method A).

8.4d. 5-(4-chlorophenyl)-3-fluoro-2-{4-[(E)-2-methyl-3-(4-methylpiperidin-1-yl)propenyl]-phenylethynyl}pyridine

Prepared according to General Method I from 70 mg (0.20 mmol) of 1-[(E)-3-(4-iodophenyl)-2-methylallyl]-4-methylpiperidine and 46 mg (0.20 mmol) of 5-(4-chlorophenyl)-2-ethynyl-3-fluoropyridine (with Pd(dppf)Cl₂ as catalyst, triethylamine as base, and THF as solvent). Yield: 4 mg (4% of theoretical); C₂₉H₂₈ClFN₂ (M=458.997); calc.: molpeak (M+H)⁺: 459/461 (Cl); found: molpeak (M+H)⁺: 459/461 (Cl); retention time HPLC: 6.2 min (method D).

### EXAMPLE 8.5

### 5-(4-methylcyclohex-1-enyl)-2-{4-[(E)-2-methyl-3-(4-methylpiperidin-1-yl)propenyl]phenylethynyl}pyridine

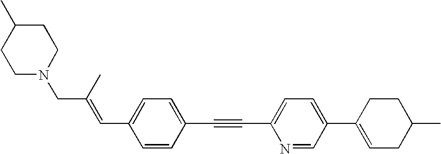

Prepared according to General Method I from 70 mg (0.20 mmol) of 1-[(E)-3-(4-iodophenyl)-2-methylallyl]-4-methylpiperidine and 39 mg (0.20 mmol) of 2-ethynyl-5-(4-methylcyclohex-1-enyl)pyridine (with Pd(dppf)Cl₂ as catalyst, triethylamine as base, and THF as solvent). Yield: 5 mg (6% of theoretical); C₃₀H₃₆N₂ (M=424.620); calc.: molpeak (M+H)⁺: 425; found: molpeak (M+H)⁺: 425; retention time HPLC: 6.1 min (method D).

### EXAMPLE 8.6

### 1-((E)-3-{4-[5-(4-chlorophenyl)-3-fluoropyridin-2-ylethynyl]phenyl}-2-methylallyl)4-methylpiperidin-4-ol

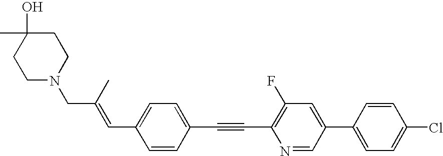

8.6a. 1-[(E)-3-(4-iodophenyl)-2-methylallyl]-4-methylpiperidin-4-ol

433 mg (3.76 mmol) of 4-methylpiperidin-4-ol is added to a solution of 550 mg (1.88 mmol) of 1-((E)-3-chloro-2-methylpropenyl)-4-iodobenzene and 0.79 mL (5.64 mmol) of triethylamine in 5 mL of DMF and the reaction mixture is stirred overnight at RT. It is evaporated down in vacuo, the residue is taken up in water and extracted exhaustively with EtOAc, and the combined organic phases are washed twice with water and dried over magnesium sulfate. After the desiccant and solvent have been eliminated, the residue is purified by chromatography (silica gel, gradient EtOAc to EtOAc/MeOH 9:1). Yield: 170 mg (24% of theoretical); C₁₆H₂₂INO (M=371.256); calc.: molpeak (M+H)^{+:} 372; found: molpeak (M+H)⁺: 372.

8.6b. 1-((E)-3-{4-[5-(4-chlorophenyl)-3-fluoropyridin-2-ylethynyl]phenyl}-2-methylallyl)-4-methylpiperidin-4-ol

Prepared according to General Method I from 85 mg (0.23 mmol) of 1-[(E)-3-(4-iodophenyl)-2-methylallyl]-4-methylpiperidin-4-ol and 53 mg (0.23 mmol) of 5-(4-chlorophenyl)-2-ethynyl-3-fluoropyridine (with Pd(dppf)Cl₂ as catalyst, triethylamine as base, and THF as solvent). Yield: 25 mg (23% of theoretical); C₂₉H₂₈ClFN₂O (M=474.997); calc.: molpeak (M+H)⁺: 475/477 (Cl); found: molpeak (M+H)⁺: 475/477 (Cl); retention time HPLC: 5.8 min (method D).

### EXAMPLE 8.7

### 4-methyl-1-((E)-2-methyl-3-{4-[5-(4-methylcyclohex-1-enyl)pyridin-2-ylethynyl]-phenyl}allyl)piperidin-4-ol

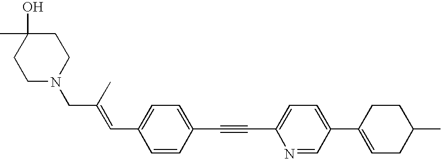

Prepared according to General Method I from 85 mg (0.23 mmol) of 1-[(E)-3-(4-iodophenyl)-2-methylallyl]4-methylpiperidin-4-ol and 45 mg (0.23 mmol) of 2-ethynyl-5-(4-methylcyclohex-1-enyl)pyridine (with Pd(dppf)Cl₂ as catalyst, triethylamine as base, and THF as solvent). Yield: 7 mg (7% of theoretical); C₃₀H₃₆N₂O (M=440.620); calc.: molpeak (M+H)⁺: 441; found: molpeak (M+H)⁺: 441; retention time HPLC: 5.7 min (method D).

### EXAMPLE 9

### 5-(4-chlorophenyl)-2-{4-[2-methyl-2-(4-methylpiperidin-1-yl)propoxy]phenyl-ethynyl}pyridine

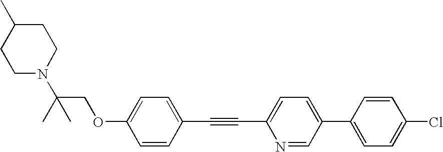 9a. ethyl 2-methyl-2-(4-methylpiperidin-1-yl)propionate

A solution of 3.72 mL (25.0 mmol) of ethyl 2-bromo-2-methylpropionate in 40 mL of 4-methylpiperidine is stirred overnight at 70° C. and then the solvent is eliminated in vacuo. The residue is taken up in EtOAc and water. The organic phase is dried over magnesium sulfate and the solvent is eliminated in vacuo. The crude product is reacted further without any further purification. Yield: 3.00 g (56.3% of theoretical); C₁₂H₂₃NO₂ (M=213.317); calc.: molpeak (M+H)⁺: 214; found: molpeak/GC-MS (M+H)⁺: 214; retention time HPLC: 3.87 min (method A).

9b. 2-methyl-2-(4-methylpiperidin-1-yl)propan-1-ol

9.20 mL (9.20 mmol) of a 1 M lithium aluminum hydride solution in THF is added dropwise at RT to a solution of 3.00 g (9.14 mmol) of ethyl 2-methyl-2-(4-methylpiperidin-1-yl)propionate in 20 mL of THF. The reaction solution is heated to 50° C. and stirred overnight. After cooling, it is diluted with ice water and the aqueous phase is extracted with diethyl ether. The organic phase is dried over magnesium sulfate and the solvent is eliminated in vacuo. The crude product is reacted further without any further purification. Yield: 2.02 g (71.0% of theoretical; content 55%); C₁₀H₂₁NO (M=171.280); calc.: molpeak (M+H)⁺: 172; found: molpeak (M+H)⁺: 172; retention time HPLC: 1.88 min (method A).

9c. 1-[2-(4-iodophenoxy)-1,1-dimethylethyl]-4-methylpiperidine

700 mg (3.18 mmol) of 4-iodophenol and 1.25 g (4.77 mmol) of triphenylphosphine are added to a solution of 1 .00 g (3.21 mmol; 55% content) of 2-methyl-2-(4-methylpiperidin-1-yl)propan-1-ol in 20 mL of DCM. 1.00 mL (4.79 mmol) of DIAD is slowly added while cooling. The reaction mixture is stirred for 2 hours at RT and water is added, the organic phase is separated off and extracted twice with water, and the combined organic phases are dried over magnesium sulfate and the solvent is eliminated in vacuo. Further purification is carried out by column chromatography on silica gel (EtOAc). Yield: 1.85 g (84.9% of theoretical; content 55%); C₁₆H₂₄INO (M=373.272); calc.: molpeak (M)⁺: 373; found: molpeak (M)⁺: 373; retention time HPLC: 6.09 min (method A).

9d. 5-(4-chlorophenyl)-2-{4-[2-methyl-2-(4-methylpiperidin-1-yl)propoxy]phenylethynyl}-pyridine

Prepared according to General Method I from 1-[2-(4-iodophenoxy)-1,1-dimethylethyl]-4-methylpiperidine (200 mg, 0.54 mmol) and 5-(4-chlorophenyl)-2-ethynylpyridine (110 mg, 0.50 mmol). Yield: 25 mg (10.1% of theoretical); C₂₉H₃₁CIN₂O (M=459.022); calc.: molpeak (M+H)⁺: 459/461 (Cl); found: molpeak (M+H)⁺: 459/461 (Cl); retention time HPLC: 5.7 min (method A).

### EXAMPLE 10

### 5-(4-chlorophenyl)-2-[4-(1-pyrrolidin-1-ylcycloprolmethoxy)phenylethynyl]-pyridine

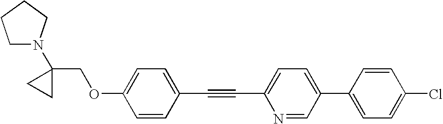 10a. 1-(4-iodophenoxymethyl)cyclopropylamine

While cooling with ice/isopropanol, 22.1 mL (72.7 mmol) of titanium (IV) isopropoxide is added dropwise to a solution of 13.8 g (65.1 mmol) of (4-iodophenoxy)acetonitrile in 250 mL of diethyl ether. Then 43.4 mL (130 mmol) of a 3M ethylmagnesium bromide solution in diethyl ether is added dropwise at 0° C. The reaction mixture is stirred for 30 minutes. Then 16.5 mL (130 mmol) of boron trifluoride-diethyl ether complex is added quickly. The reaction mixture is stirred for 30 minutes and then, while cooling with ice, 200 mL of a 1 M sodium hydroxide solution is added. After an hour, the aqueous phase is extracted with 300 mL of diethyl ether. The organic phase is dried over sodium sulfate and the solvent is reduced in vacuo to 400 mL. The organic phase is washed with 180 mL of saturated Na₂SO₃ solution and washed twice with 400 mL of 0.05 M of hydrochloric acid solution. The aqueous phase is made alkaline with 30% sodium hydroxide solution and extracted with 400 mL of DCM. The combined organic extracts are dried over magnesium sulfate and the solvent is eliminated in vacuo. The crude product is reacted further without any further purification. Yield: 7.67 g (48.7% of theoretical); C₁₀H₁₂BrNO (M=242.112); calc.: molpeak (M+H)⁺: 242/244 (Br); found: molpeak (M+H)⁺: 242/244 (Br); retention time HPLC: 4.96 min (method A).

10b. 1-[1-(4-bromophenoxymethyl)cyclopropyl]pyrrolidine

0.50 mL of 1,4-dibromobutane and 1.14 g (8.26 mmol) of potassium carbonate (K₂CO₃) are added to a solution of 1.00 g (4.13 mmol) of 1-(4-iodophenoxymethyl)cyclopropylamine in 50 mL of DMF. The reaction mixture is stirred for 24 hours at 80° C. The solvent is eliminated in vacuo and the residue is combined with 100 mL of water and 200 mL of EtOAc. The organic phase is separated off, dried over sodium sulfate and the solvent is eliminated in vacuo. The crude product is reacted further without any further purification. Yield: 1.33 g (108.6% of theoretical); C₁₄H₁₈BrNO (M=296.203); calc.: molpeak (M+H)⁺: 296/298 (Br); found: molpeak (M+H)⁺: 296/298 (Br); retention time HPLC: 6.03 min (method A).

10c. 1-[1-(4-iodophenoxymethyl)cyclopropyl]pyrrolidine

Prepared according to General Method II from 1-[1-(4-bromophenoxymethyl)cyclopropyl]-pyrrolidine (1.22 g, 4.12 mmol). Yield: 473 mg (33.5% of theoretical); C₁₄H₁₈INO (M=343.209); calc.: molpeak (M+H)⁺: 344; found: molpeak (M+H)⁺: 344; retention time HPLC: 6.17 min (method A).

10d. 5-(4-chlorophenyl)-2-[4-(1-pyrrolidin-1-ylcyclopropylmethoxy)phenylethynyl]pyridine

Prepared according to General Method I from 1-[1-(4-iodophenoxymethyl)cyclopropyl]-pyrrolidine (100 mg, 0.29 mmol) and 5-(4-chlorophenyl)-2-ethynylpyridine (78 mg, 0.36 mmol). Yield: 15 mg (12.0% of theoretical); C₂₇H₂₅ClN₂O (M=428.953); calc.: molpeak (M+H)⁺: 429/431 (Cl); found: molpeak (M+H)⁺: 429/431 (Cl); retention time HPLC: 8.28 min (method A).

### EXAMPLE 11.1

### (R)-5-(4-chlorophenyl)-2-{4-[2-(4-methylpiperidin-1-yl)propoxy]phenylethynyl}pyridine

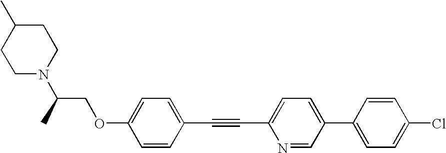 11.1a. (R)-2-[2-(4-iodophenoxy)-1-methylethyl]isoindol-1,3-dione

5.40 g (26.3 mmol) of (R)-2-(2-hydroxy-1-methylethyl)isoindol-1,3-dione in 40 mL of THF is added to a solution of 8.80 g (30.0 mmol) of 4-iodophenol and 10.5 g (40.0 mmol) of triphenylphosphine in 180 mL of THF in a nitrogen atmosphere. The reaction solution is cooled to 0° C. and 7.93 mL (40.0 mmol) of DIAD in 20 mL of THF is added, the ice bath is removed, and the mixture is stirred overnight at RT. Another 10.5 g (40.0 mmol) of triphenylphosphine and 6.0 mL (30.3 mmol) of DIAD are added and the mixture is stirred for a further 4 hours. The solvent is eliminated in vacuo and the residue is taken up in EtOAc. The organic phase is washed twice with semisaturated sodium carbonate solution, dried over magnesium sulfate, and the solvent is eliminated in vacuo. The residue is combined with TBME and DIPE. After filtration, the solvent is eliminated in vacuo. Further purification is carried out by column chromatography on silica gel (PE/EtOAc 9:1 towards PE/EtOAc 6:4). The residue is taken up in EtOAc and washed twice with 1M sodium hydroxide solution, and the organic phase is dried over magnesium sulfate and the solvent is eliminated in vacuo. Yield: 6.60 g (61.6% of theoretical); C₁₇H₁₄INO₃ (M=407.202); calc.: molpeak (M+H)⁺: 408; found: molpeak (M+H)⁺: 408; retention time HPLC: 6.41 min (method B).

11.1 b. (R)-2-(2-{4-[5-(4-chlorophenyl)pyridin-2-ylethynyl]phenoxy}-1-methylethyl)isoindol-1,3-dione

Prepared according to General Method I from (R)-2-[2-(4-iodophenoxy)-1-methylethyl]isoindol-1,3-dione (1.00 g, 2.46 mmol) and 5-(4-chlorophenyl)-2-ethynylpyridine (643 mg, 2.80 mmol). Yield: 700 mg (57.8% of theoretical); C₃₀H₂₁ClN₂O₃ (M=492.952); calc.: molpeak (M+H)⁺:493/495 (Cl); found: molpeak (M+H)⁺: 493/495 (Cl); R_{f} value: 0.60 (silica gel, PE/EtOAc 6:4).

11.1c. (R)-2-{4-[5-(4-chlorophenyl)pyridin-2-ylethynyl]phenoxy}-1-methylethylamine 3.0 mL of methylarnine (40% in water) is added to a solution of 700 mg (1.42 nmuol) of (R)-2-(2-{4-[5-(4-chlorophenyl)pyridin-2-ylethynyl]phenoxy}-1-methylethyl)isoindol-1,3-dione in 3.0 mL of toluene and the reaction solution is stirred overnight. The reaction mixture is combined with 80 mL of DCM, extracted three times with water, dried over magnesium sulfate, and the solvent is eliminated in vacuo. The residue is stirred with TBME and, after filtration, dried in the air. The succinimide has been only partially opened. Therefore the residue is combined with 5.0 mL of 40% methylamine and 5.0 mL of toluene and stirred for 3 days in a sealed vessel at 60° C. The reaction mixture is diluted with 120 mL of DCM. The organic phase is extracted three times with water, dried over magnesium sulfate, and the solvent is eliminated in vacuo. The residue is stirred with TBME and EtOAc and, after filtration, dried in the air. Yield: 250 mg (48.5% of theoretical); C₂₂H₁₉ClN₂O (M=362.852); calc.: molpeak (M+H)⁺: 363/365 (Cl); found: molpeak (M+H)⁺: 363/365 (Cl); R_{f} value: 0.08 (silica gel, DCM/MeOH 9:1).

11.1 d. (R)-5-(4-chlorophenyl)-2-{4-[2-(4-methylpiperidin-1-yl)propoxy]phenylethynyl}-pyridine

61.0 mg (0.25 mmol) of 1,5-dibromo-3-methylpentane and 75.0 mg (0.54 mmol) of potassium carbonate are added to a solution of 80.0 mg (0.22 mmol) of (R)-2-{4-[5-(4-chlorophenyl)pyridin-2-ylethynyl]phenoxy}-1-methylethylamine in 2.00 mL of DMF. The mixture is shaken overnight at 50° C. After cooling, a precipitate is formed which is combined with some water. After filtration, the mixture is washed with water and isopropanol. Further purification is carried out by column chromatography on silica gel (DCM/MeOH/NH₃ 9:1:0.1). Yield: 24 mg (25.0% of theoretical); C₂₈H₂₉ClN₂O (M=444.995); calc.: molpeak (M+H)⁺: 445/447 (Cl); found: molpeak (M+H)⁺: 445/447 (Cl); R_{f} value: 0.12 (silica gel, DCM/MeOH/NH₃ 95:5:0.5).

### EXAMPLES 11.2 AND 11.3

### EXAMPLE 11.2

### (R)-(2-{4-[5-(4-chlorophenyl)pyiridin-2-ylethynyl]phenoxy}-1-methylethyl)-cyclopropylmethylamine

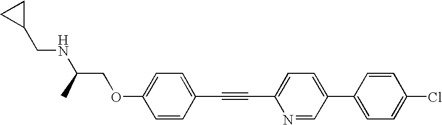

### EXAMPLE 11.3

### (R)-(2-{4-[5-(4-chlorophenyl)pyridin-2-ylethvnvyl]phenoxy}-1-methylethyl)-bis(cyclopropylmethyl)amine

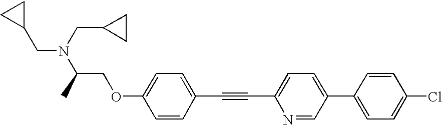

Examples 11.2 and 11.3: A solution of 190 mg (0.52 mmol) of (R)-2-{4-[5-(4-chlorophenyl)pyridin-2-ylethynyl]phenoxy}-1-methylethylamine and 0.06 mL (0.80 mmol) of cyclopropanecarboxaldehyde in 75 mL of THF is stirred for 1 hour at RT and then 339 mg (1.60 mmol) of NaBH(OAc)₃ and 0.01 mL (0.25 mmol) of glacial acetic acid are added. The reaction mixture is stirred for 2 hours at RT. Some of the solvent is eliminated in vacuo and the residue is diluted with 50 mL of EtOAc and 30 mL of saturated sodium bicarbonate solution. The organic phase is extracted with water, dried over magnesium sulfate, and the solvent is eliminated in vacuo. Further purification is carried out by column chromatography on silica gel (DCM towards DCM/MeOH/NH₃ 9:1:0.1). The desired fractions are evaporated down and the respective residues are triturated with TBME and DIPE and, after filtration, dried in the air.

11.2: (R)-(2-{4-[5-(4-chlorophenyl)pyridin-2-ylethynyl]phenoxy}-1-methylethyl)cyclopropyl-methylamine. Yield: 27 mg (12.4% of theoretical); C₂₆H₂₅ClN₂O (M=416.942); calc.: molpeak (M+H)⁺: 417/419 (Cl); found: molpeak (M+H)⁺: 417/419 (Cl); Rf value: 0.56 (silica gel, DCM/MeOH/NH₃ 9:1:0.1).

11.3: (R)-(2-{4-[5-(4-chlorophenyl)pyridin-2-ylethynyl]phenoxy}-1-methylethyl)-bis(cyclopropylmethyl)amine.

Yield: 58 mg (23.5% of theoretical); C₃₀H₃₁ClN₂O (M=471.033); calc.: molpeak (M+H)⁺: 471/473 (Cl); found: molpeak (M+H)⁺: 471/473 (Cl); R_{f} value: 0.87 (silica gel, DCM/MeOH/NH₃ 9:1:0.1).

### EXAMPLE 12.1

### (S)-5-(4-chlorophenyl)-2-{4-[2-(4-methylpiperidin-1-yl)propoxy]phenylethvnyl}-pyridine

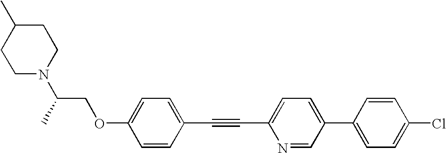 12.1a. (S)-2-[2-(4-iodophenoxy)-1-methylethyl]isoindol-1,3-dione

Analogously to 11.1a the product is obtained from 6.30 g (30.7 mmol) of (S)-2-(2-hydroxy-1-methylethyl)isoindol-1,3-dione, 7.04 g (32.0 mmol) of 4-iodophenol, 23.6 g (90.0 mmol) of triphenylphosphine, and 17.8 mL (90.0 mmol) of DIAD. Yield: 2.50 g (20.0% of theoretical); C₁₇H₁₄INO₃ (M=407.202); calc.: molpeak (M+H)⁺: 408; found: molpeak (M+H)⁺: 408; retention time HPLC: 6.41 min (method B).

12.1b. (S)-2-(2-{4-[5-(4-chlorophenyl)pyridin-2-ylethynyl]phenoxy}-1-methylethyl)isoindol-1,3-dione

Prepared according to General Method I from (R)-2-[2-(4-iodophenoxy)-1-methylethyl]isoindol-1,3-dione (2.50 g, 6.14 mmol) and 5-(4-chlorophenyl)-2-ethynylpyridine (1.49 g, 6.50 mmol). Yield: 1.30 g (43.0% of theoretical); C₃₀H₂₁ClN₂O₃ (M=492.952); calc.: molpeak (M+H)⁺:493/495 (Cl); found: molpeak (M+H)⁺: 493/495 (Cl); retention time HPLC: 7.18 min (method A).

12.1c. (S)-2-{4-[5-(4-chlorophenyl)pyridin-2-ylethynyl]phenoxy}-1-methylethylamine

Analogously to 11.1c the product is obtained from 1.30 g (2.64 mmol) of (S)-2-(2-{4-[5-(4-chlorophenyl)pyridin-2-ylethynyl]phenoxy}-1-methylethyl)isoindol-1,3-dione and 6 mL of 40% methylamine solution in water. Yield: 650 mg (67.9% of theoretical); C₂₂H₁₉ClN₂O (M=362.852); calc.: molpeak (M+H)⁺: 363/365 (Cl); found: molpeak (M+H)⁺: 363/365 (Cl); retention time HPLC: 4.87 min (method B).

12.1d. (S)-5-(4-chlorophenyl)-2-{4-[2-(4-methylpiperidin-1-yl)propoxy]phenylethynyl}pyridine

Analogously to 11.1d the product is obtained from 100 mg (0.28 mmol) of (S)-2-{4-[5-(4-chlorophenyl)pyridin-2-ylethynyl]phenoxy}-1-methylethylamine and 76 mg (0.31 mmol) of 1,5-dibromo-3-methylpentane. Yield: 20 mg (16.3% of theoretical); C₂₈H₂₉ClN₂O (M=444.995); calc.: molpeak (M+H)⁺: 445/447 (Cl); found: molpeak (M+H)⁺: 445/447 (Cl); R_{f} value: 0.48 (silica gel, DCM/MeOH/NH₃ 9:1:0.1).

### EXAMPLES 12.2 AND 12.3

### EXAMPLE 12.2

### (S)-(2-{4-[5-(4-chlorophenyl)pyridin-2-ylethynyl]phenoxy}-1-methylethyl)-cyclopropylmethylamine

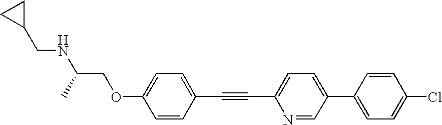

### EXAMPLE 12.3

### (S)-(2-{4-[5-(4-chlorophenyl)pyridin-2-ylethynyl]phenoxy}-1-methylethyl)-bis(cyclopropylmethyl)amine

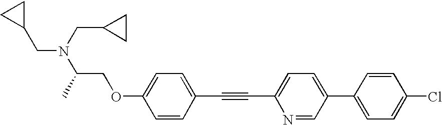

Examples 12.2 and 12.3: A solution of 290 mg (0.80 mmol) of (S)-2-{4-[5-(4-chlorophenyl)pyridin-2-ylethynyl]phenoxy}-1-methylethylamine and 0.09 mL (1.20 mmol) of cyclopropanecarboxaldehyde in 75 mL of THF is stirred for 1 hour at RT. Then 509 mg (2.40 mmol) of NaBH(OAc)₃ and 0.02 mL (0.40 mmol) of glacial acetic acid are added. The reaction mixture is stirred for 2 hours at RT. Some of the solvent is eliminated in vacuo and the residue is diluted with 50 mL of EtOAc and 30 mL of saturated sodium bicarbonate solution. The organic phase is washed with water, dried over magnesium sulfate, and the solvent is eliminated in vacuo. Further purification is carried out by column chromatography on silica gel (DCM towards DCM/MeOH/NH₃ 9:1:0.1). The desired fractions are evaporated down and the respective residues are triturated with TBME and after filtration dried in the air.

12.2: (S)-(2-{4-[5-(4-chlorophenyl)pyridin-2-ylethynyl]phenoxy}-1-methylethyl)cyclopropylmethylamine.

Yield: 56 mg (16.8% of theoretical); C₂₆H₂₅ClN₂O (M=416.942); calc.: molpeak (M+H)⁺: 417/419 (Cl); found: molpeak (M+H)⁺: 417/419 (Cl); R_{f} value: 0.48 (silica gel, DCMIMeOH/NH₃ 9:1:0.1).

12.3: (S)-(2-{4-[5-(4-chlorophenyl)pyridin-2-ylethynyl]phenoxy}-1-methylethyl)-bis(cyclopropylmethyl)amine.

Yield: 32 mg (9.0% of theoretical); C₃₀H₃₁ClN₂O (M=471.033); calc.: molpeak (M+H)⁺: 471/473 (Cl); found: molpeak (M+H)⁺: 471/473 (Cl); R_{f} value: 0.82 (silica gel, DCM/MeOH/NH₃ 9:1:0.1).

### EXAMPLE 13

### (R)-5-(4-chlorophenyl)-2-{4-[3-methyl-2-(4-methylpiperidin-1-yl)butoxy]phenyl-ethnynyl}pyridine

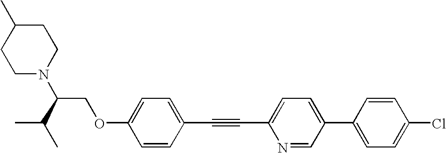 13a. (R)-3-methyl-2-(4-methylpiperidin- 1-yl)butan-1-ol

1.32 g (5.40 mmol) of 1,5-dibromo-3-methylpentane and 1.55 g (11.2 mmol) of potassium carbonate are added to a solution of 0.50 g (4.85 mmol) of R—(−)-2-amino-3-methyl-1-butanol in 10 mL of DMF. The reaction mixture is stirred ovemight at 50° C. After cooling, it is diluted with 50 mL of EtOAc and washed three times with semisaturated sodium bicarbonate solution. The organic phase is dried over magnesium sulfate and the solvent is eliminated in vacuo. The residue is filtered through a silica gel bed (DCM towards DCM/MeOH 8:2). Yield: 350 mg (39.0% of theoretical); C₁₁H₂₃NO (M=185.306); calc.: molpeak (M+H)⁺: 186; found: molpeak (M+H)⁺: 186; R_{f} value: 0.30 (silica gel, DCM/MeOH 9:1).

13b. (R)-1-[1-(4-bromophenoxymethyl)-2-methylpropyl]-4-methylpiperidine

0.54 g (1.90 mmol) of 4-bromoiodophenol, 1.24 g (3.80 mmol) of cesium carbonate, 68.0 mg (0.38 mmol) of 1,10-phenanthroline, and 36.0 mg (0.19 mmol) of Cul are added to a solution of 0.35 g (1.89 mmol) of (R)-3-methyl-2-(4-methylpiperidin-1-yl)butan-1-ol in 3.0 mL of toluene. The reaction mixture is shaken for 36 hours at 110° C. After cooling, it is diluted with 40 mL of EtOAc and washed twice with 30 mL of water. The organic phase is dried over magnesium sulfate and the solvent is eliminated in vacuo. Further purification is carried out by column chromatography on silica gel (DCM towards DCM/MeOH 9:1). Yield: 100 mg (16.0% of theoretical); C₁₇H₂₆BrNO (M=340.298); calc.: molpeak (M+H)⁺: 340/342 (Br); found: molpeak (M+H)⁺: 340/342 (Br); retention time HPLC: 4.93 min (method B).

13c. (R)-1-[1-(4-iodophenoxymethyl)-2-methylpropyl]-4-methylpiperidine

Prepared according to General Method II from (R)-1-[1-(4-bromophenoxymethyl)-2-methylpropyl]4-methylpiperidine (0.10 g, 0.29 mmol). Yield: 60 mg (53.0% of theoretical); C₁₇H₂₆INO (M=387.299); calc.: molpeak (M+H)⁺: 388; found: molpeak (M+H)⁺: 388; retention time HPLC: 7.54 min (method A).

13d. (R)-5-(4-chlorophenyl)-2-{4-[3-methyl-2-(4-methylpiperidin-1-yl)butoxy]phenyl-ethynyl}pyridine

Prepared according to General Method I from (R)-1-[1-(4-iodophenoxymethyl)-2-methylpropyl]-4-methylpiperidine (60.0 mg, 0.16 mmol) and 5-(4-chlorophenyl)-2-ethynylpyridine (35.6 mg, 0.16 mmol). Yield: 4.5 mg (6.0% of theoretical, content: min. 50%); C₃₀H₃₃CIN₂O (M=473.049); calc.: molpeak (M+H)⁺: 473/475 (Cl); found: molpeak (M+H)⁺: 473/475 (Cl); retention time HPLC: 9.65 min (method A).

### Example 14

### (S)-5-(4-chlorophenyl)-2-{4-[3-methyl-2-(4-methylpiperidin-1-yl)butoxy]phenyl-ethynyl}pyridine

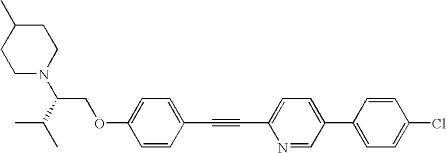 14a. (S)-3-methyl-2-(4-methylpiperidin-1-yl)butan-1-ol

Analogously to 13a the product is obtained from 1.00 g (9.69 mmol) of (S)-(+)-2-amino-3-methyl-1-butanol, 2.64 g (10.8 mmol) of 1,5-dibromo-3-methylpentane, 3.10 g (22.4 mmol) of potassium carbonate, and 20 mL of DMF. Yield: 2.20 g (98.0% of theoretical, 80%); C₁₁H₂₃NO (M=185.306); calc.: molpeak (M+H)⁺: 186; found: molpeak (M+H)⁺: 186; R_{f} value: 0.30 (silica gel, DCM/MeOH 9:1).

14b. (S)-1-[1-(4-bromophenoxymethyl)-2-methylpropyl]-4-methylpiperidine

Analogously to 13b the product is obtained from 1.1 g (4.75 mmol, 80%) of (S)-3-methyl-2-(4-methylpiperidin-1-yl)butan-1-ol, 1.34 g (4.75 mmol) of 4-bromoiodophenol, 3.10 g (9.50 mmol) of cesium carbonate, 0.17 g (0.95 mmol) of 1,10-phenanthroline, 0.09 g (0.48 mmol) of CuI, and 10 mL of toluene. Yield: 0.15 g (9.0% of theoretical); C₁₇H₂₆BrNO (M=340.298); calc.: molpeak (M+H)⁺: 340/342 (Br); found: molpeak (M+H)⁺: 340/342 (Br); retention time HPLC: 7.4 min (method A).

14c. (S)-1-[1-(4-iodophenoxymethyl)-2-methylpropyl]-4-methylpiperidine

Prepared according to General Method II from (S)-1-[1-(4-bromophenoxymethyl)-2-methylpropyl]-4-methylpiperidine (0.15 g, 0.44 mmol). Yield: 0.10 g (59.0% of theoretical); C₁₇H₂₆INO (M=387.299); calc.: molpeak (M+H)⁺: 388; found: molpeak (M+H)⁺: 388; retention time HPLC: 4.96 min (method D).

14d. (S)-5-(4-chlorophenyl)-2-{4-[3-methyl-2-(4-methylpiperidin-1-yl)butoxy]phenyl-ethynyl}pyridine

Prepared according to General Method I from (S)-1-[1-(4-iodophenoxymethyl)-2-methylpropyl]-4-methylpiperidine (100 mg, 0.26 mmol) and 5-(4-chlorophenyl)-2-ethynylpyridine (61.0 mg, 0.28 mmol). Yield: 20.0 mg (13.0% of theoretical, 80%); C₃₀H₃₃CIN₂O (M=473.049); calc.: molpeak (M+H)⁺: 473/475 (Cl); found: molpeak (M+H)⁺: 473/475 (Cl); retention time HPLC: 6.1 min (method D).

### EXAMPLE 15.1

### (E)-(R)-3-{4-[5-(4-chlorophenyl)pyridin-2-ylethynyl]phenyl}-1-methylbut-2-enylamine

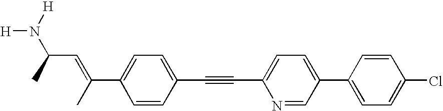 15.1a. tert-butyl [(E)-(R)-3-(4-bromophenyl)- 1-methylbut-2-enyl]carbamate

27.7 mL (27.7 mmol, 1M in n-hexane) lithium bis(trimethylsilyl)amide solution is added dropwise at RT within 20 minutes, while cooling gently with water, to a suspension of 14.6 g (27.7 mmol) of [1-(4-bromophenyl)ethyl]triphenylphosphonium bromide in 250 mL of diethyl ether. The mixture is stirred for 4 hours and cooled to 0° C. A solution of 4.80 g (27.7 mmol) of tert-butyl ((R)-1-methyl-2-oxoethyl)carbamate in 50 mL of diethyl ether is added dropwise. The mixture is stirred for a further 20 hours at RT. The reaction mixture is filtered through CELITE® filter aid and the solvent is eliminated in vacuo. The residue is purified through a gravity silica gel column (cyclohexane/EtOAc 4:1). Yield: 1.90 g (20.1% of theoretical);

C₁₆H₂₂BrNO₂ (M=340.255); calc.: molpeak (M+H)⁺: 340/342 (Br); found: molpeak (M+H)⁺: 340/342 (Br); R_{f} value: 0.56 (silica gel, cyclohexane/EtOAc 4:1).

15.1b. tert-butyl [(E)-(R)-3-(4-iodophenyl)- 1-methylbut-2-enyl]carbamate

Prepared according to General Method II from tert-butyl [(E)-(R)-3-(4-bromophenyl)-1-methylbut-2-enyl]carbamate (1.90 g, 5.58 mmol). Yield: 1.87 g (86.7% of theoretical); C₁₆H₂₂INO₂ (M=387.256); calc.: molpeak (M+H)⁺: 388; found: molpeak (M+H)⁺: 388; retention time HPLC: 6.71 min (method B).

15.1c. tert-butyl ((E)-(R)-3-{4-[5-(4-chlorophenyl)pyridin-2-ylethynyl]phenyl}-1-methylbut-2-enyl)carbamate

Prepared according to General Method I from tert-butyl [(E)-(R)-3-(4-iodophenyl)-1-methylbut-2-enyl]carbamate (1.87 g, 4.84 mmol) and 5-(4-chlorophenyl)-2-ethynylpyridine (1.35 g, 5.80 mmol). Yield: 1.36 g (59.4% of theoretical); C₂₉H₂₉ClN₂O₂ (M=473.006); calc.: molpeak (M+H)⁺: 473/475 (Cl); found: molpeak (M+H)⁺: 473/475 (Cl); R_{f} value: 0.09 (silica gel, 366 nm, DCM).

15.1d. (E)-(R)-3-{4-[5-(4-chlorophenyl)pyridin-2-ylethynyl]phenyl}-1-methylbut-2-enylamine

6.59 mL of trifluoroacetic acid is added to a solution of 1.36 g (2.87 mmol) of tert-butyl ((E)-(R)-3-{4-[5-(4-chlorophenyl)pyridin-2-ylethynyl]phenyl}-1-methylbut-2-enyl)carbamate in 70 mL of DCM and the mixture is stirred for 15 hours at RT. The reaction mixture is made alkaline with 15% sodium hydroxide solution. The organic phase is dried over sodium sulfate and the solvent is eliminated in vacuo. Further purification is carried out by column chromatography on silica gel (DCM/MeOH/NH₃ 19:1:0.1). Yield: 610 mg (57% of theoretical); C₂₄H₂₁ClN₂ (M=372.890); calc.: molpeak (M+H)⁺: 373/375 (Cl); found: molpeak (M+H)⁺: 373/375 (Cl); R_{f} value: 0.25 (silica gel, 366 nm, DCM/MeOH/NH₃ 9:1:0.1).

### EXAMPLE 15.2

### (E)-(R)-5-(4-chlorophenyl)-2-{4-[1-methyl-3-(4-methylpiperidin-1-yl)but-1-enyl]phenylethynyl}pyridine

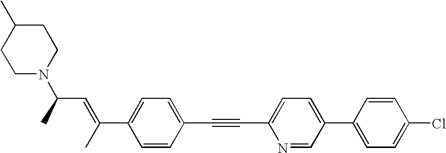

58.3 mg (0.38 mmol) of 1,5-dibromo-3-methylpentane and 0.13 mL (0.75 mmol) of ethyldiisopropylamine are added to a solution of 70.0 mg (0.19 mmol) of (E)-(R)-3-{4-[5-(4-chlorophenyl)pyridin-2-ylethynyl]phenyl}-1-methylbut-2-enylamine in 1.7 mL of DMF and the mixture is shaken for 3.5 hours at 80° C. The solvent is eliminated in vacuo. The residue is taken up in 20 mL of DCM and washed with 10 mL of semisaturated sodium bicarbonate solution. The organic phase is dried over sodium sulfate and the solvent is eliminated in vacuo. Further purification is carried out by column chromatography on silica gel (DCM/MeOHINH₃ 99:1:0.1). The residue is stirred with DIPE. Yield: 24.4 mg (29% of theoretical); C₃₀H₃₁ClN₂ (M=455.033); calc.: molpeak (M+H)⁺: 455/457 (Cl); found: molpeak (M+H)⁺: 455/457 (Cl); R_{f} value: 0.20 (silica gel, DCM/MeOH/NH₃ 9: 1:0.1).

### EXAMPLES 15.3 AND 15.4

### EXAMPLE 15.3

### ((E)-(R)-3-{4-[5-(4-chlorophenyl)pyridin-2-ylethynyl]phenyl}-1-methylbut-2-enyl)cyclopropylmethylamine

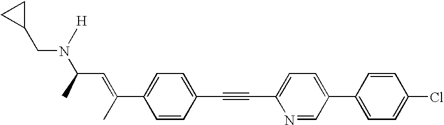

### EXAMPLE 15.4

### : ((E)-(R)-3-{4-[5-(4-chlorophenyl)pyridin-2-ylethynyl]phenyl}-1-methylbut-2-enyl)-bis(cyclopropylmethyl)amine

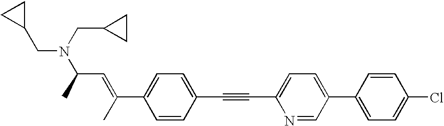

### Examples 15.3 and 15.4: A solution of 325 mg (0.87 mmol) of (E)-(R)-3-{4-[5-(4-chlorophenyl)pyridin-2-ylethynyl]phenyl}-1-methylbut-2-enylamine and 0.07 mL (0.87 mmol) of cyclopropanecarboxaldehyde in 25 mL of THF is stirred for 1 hour at RT. Then 739 mg (3.49 mmol) of NaBH(OAc)₃ and 0.20 mL (3.49 mmol) of glacial acetic acid are added. The reaction mixture is stirred for 2.5 hours at RT. The solvent is eliminated in vacuo and the residue is diluted with 50 mL of EtOAc and 30 mL of semisaturated potassium carbonate solution. The organic phase is dried over sodium sulfate and the solvent is eliminated in vacuo. Further purification is carried out by column chromatography on silica gel (DCM/MeOH/NH₃ 19:1:0.1). The desired fractions are evaporated down and the respective residues are triturated with DIPE and after filtration dried in the air.

15.3: ((E)-(R)-3-{4-[5-(4-chlorophenyl)pyridin-2-ylethynyl]phenyl}-1-methylbut-2-enyl)-cyclopropylmethylamine. Yield: 190 mg (51% of theoretical); C₂₈H₂₇ClN₂ (M=426.980); calc.: molpeak (M+H)⁺: 427/429 (Cl); found: molpeak (M+H)⁺: 427/429 (Cl); R_{f} value: 0.56 (silica gel, DCM/MeOH/NH₃ 19:1:0.1).

15.4: ((E)-(R)-3-{4-[5-(4-chlorophenyl)pyridin-2-ylethynyl]phenyl}-1-methylbut-2-enyl)-bis(cyclopropylmethyl)amine.

Yield: 11.6 mg (3% of theoretical); C₃₂H₃₃ClN₂ (M=481.071); calc.: molpeak (M+H)⁺: 481/483 (Cl); found: molpeak (M+H)⁺: 481/483 (Cl); R_{f} value: 0.83 (silica gel, DCM/MeOH/NH₃ 19:1:0. 1).

### EXAMPLE 15.5

### ((E)-(R)-3-{4-[5-(4-chlorophenyl)pyridin-2-ylethynyl]phenyl}-1-methylbut-2-enyl)cyclopropylmethylpropylamine

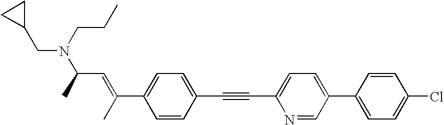

0.06 mL (0.70 mmol) of 1-bromopropane and 0.13 mL (0.74 mmol) of ethyldiisopropylamine are added to a solution of 75.0 mg (0.18 mmol) of ((E)-(R)-3-{4-[5-(4-chlorophenyl)pyridin-2-ylethynyl]phenyl}-1-methylbut-2-enyl)cyclopropylmethylamine in 1.5 mL of DMF and shaken for 22 hours at 60° C. The reaction mixture is purified by preparative HPLC-MS. The corresponding fractions are evaporated down in vacuo and combined with 10 mL of semisaturated sodium bicarbonate solution and 20 mL of DCM. The organic phase is dried over sodium sulfate and the solvent is eliminated in vacuo. Further purification is carried out by stirring with DIPE. Yield: 10.0 mg (12% of theoretical); C₃₁H₃₃ClN₂ (M=469.060); calc.: molpeak (M+H)⁺: 469/471 (Cl); found: molpeak (M+H)⁺: 469/471 (Cl); retention time HPLC: 9.15 min (method A).

### EXAMPLE 15.6

### (E)-(R)-5-(4-chlorophenyl)-2-[4-(1-methyl-3-pyrrolidin-1-ylbut-1-enyl)phenyl-ethynyl]pyridine

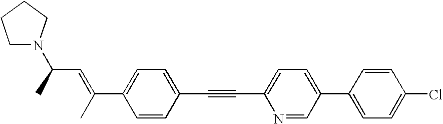

0.09 mL (0.75 mmol) of 1,4-dibromobutane and 0.13 mL (0.75 mmol) of ethyldiisopropylamine are added to a solution of 70.0 mg (0.19 mmol) of (E)-(R)-3-{4-[5-(4-chlorophenyl)pyridin-2-ylethynyl]phenyl}-1-methylbut-2-enylamine in 1.7 mL of DMF and shaken for 3.5 hours at 60° C. The reaction mixture is combined with 20 mL of semisaturated sodium bicarbonate solution and 30 mL of EtOAc. The organic phase is dried over sodium sulfate and the solvent is eliminated in vacuo. Further purification is carried out by column chromatography on silica gel (DCM/MeOH/NH₃ 19:1:0.1). The desired fractions are evaporated down and stirred with PE. Yield: 11.7 mg (15% of theoretical); C₂₈H₂₇ClN₂ (M=426.980); calc.: molpeak (M+H)⁺: 427/429 (Cl); found: molpeak (M+H)⁺: 427/429 (Cl); R_{f} value: 0.32 (silica gel, 366 nm, DCM/MeOH/NH₃ 9:1:0.1).

### EXAMPLE 16.1

### (E)-(S)-3-{4-[5-(4-chlorophenyl)pyridin-2-ylethynyl]phenyl}-1-methylbut-2-enylamine

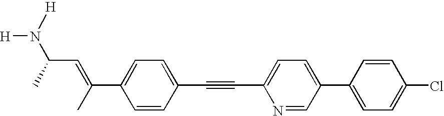 16.1a. tert-butyl [(E)-(S)-3-(4-bromophenyl)- 1-methylbut-2-enyl]carbamate

54.0 mL (54.0 mmol, 1M in THF) of lithium bis(trimethylsilyl)amide solution is slowly added dropwise to a suspension of 28.4 g (54.0 mmol) of [1-(4-bromophenyl)ethyl]-triphenylphosphonium bromide in 500 mL of diethyl ether while cooling with ice. The mixture is stirred for a further 3 hours. A solution of 9.49 g (54.8 mmol) of tert-butyl ((S)-1-methyl-2-oxoethyl)carbamate in 100 mL of diethyl ether is added dropwise. The mixture is stirred for a further 20 hours at RT. The reaction mixture is filtered and the solvent is eliminated in vacuo. The residue is purified through a gravity silica gel column (cyclohexane/EtOAc 4:2). Yield: 1.29 g (6.9% of theoretical); C₁₆H₂₂BrNO₂ (M=340.255); calc.: molpeak (M+H)⁺: 340/342 (Br); found: molpeak (M+H)⁺: 340/342 (Br); retention time HPLC: 10.71 min (method A).

16.1b. tert-butyl [(E)-(S)-3-(4-iodophenyl)- 1-methylbut-2-enyl]carbamate

Prepared according to General Method II from tert-butyl [(E)-(S)-3-(4-bromophenyl)-1-methylbut-2-enyl]carbamate (0.77 g, 2.26 mmol). Yield: 0.75 g (85.8% of theoretical); C₁₆H₂₂INO₂ (M=387.256); retention time HPLC: 6.82 min (method B).

16.1c. tert-butyl ((E)-(s)-3-{4-[5-(4-chlorophenyl)pyridin-2-ylethynyl]phenyl}-1-methylbut-2-enyl)carbamate

Prepared according to General Method I from tert-butyl [(E)-(S)-3-(4-iodophenyl)-1-methylbut-2-enyl]carbamate (1.28 g, 3.30 mmol) and 5-(4-chlorophenyl)-2-ethynylpyridine (0.84 g, 3.63 mmol). Yield: 0.43 g (27.6% of theoretical); C₂₅H₂₂ClNO (M=473.006); calc.: molpeak (M+H)⁺: 473/475 (Cl); found: molpeak (M+H)⁺: 473/475 (Cl); R_{f} value: 0.07 (silica gel, 366 nm, DCM).

16.1d. (E)-(S)-3-{4-[5-(4-chlorophenyl)pyridin-2-ylethynyl]phenyl}-1-methylbut-2-enylamine

1.98 mL (25.9 mmol) of trifluoroacetic acid is added to a solution of 0.43 g (0.91 mmol) of tert-butyl ((E)-(S)-3-{4-[5-(4-chlorophenyl)pyridin-2-ylethynyl]phenyl}-1-methylbut-2-enyl)-carbamate in 25 mL of DCM. The mixture is stirred for 17 hours at RT and then made alkaline with 15% sodium hydroxide solution. The organic phase is dried over sodium sulfate and the solvent is eliminated in vacuo. Further purification is carried out by column chromatography on silica gel (DCM/MeOH/NH₃ 19:1:0.1 towards DCM/MeOH/NH₃ 9:1:0.1). Yield: 520 mg (53.6% of theoretical); C₂₄H₂₁ClN₂ (M=372.890); calc.: molpeak (M+H)⁺: 373/375 (Cl); found: molpeak (M+H)⁺: 373/375 (Cl); R_{f} value: 0.31 (silica gel, 366 nm, DCM/MeOH/NH₃ 9:1:0.1).

### EXAMLE 16.2

### (E)-(S)-5-(4-chlorophenyl)-2-{4-[1-methyl-3-(4-methylpiperidin-1-yl)but-1-enyl]phenylethynyl}pyridine

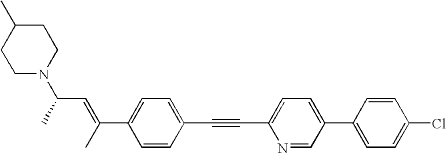

37.0 mg (0.27 mmol) of potassium carbonate and 24.8 mg (0.16 mmol) of 1,5-dibromo-3-methylpentane are added to a solution of 50.0 mg (0.13 mmol) of (E)-(S)-3-{4-[5-(4-chlorophenyl)pyridin-2-ylethynyl]phenyl}-1-methylbut-2-enylamine in 1.4 mL of DMF and shaken for 21 hours at 70° C. The solvent is eliminated in vacuo. The residue is taken up in 20 mL of EtOAc and washed with 10 mL of semisaturated sodium bicarbonate solution. The organic phase is dried over sodium sulfate and the solvent is eliminated in vacuo. Further purification is carried out by column chromatography on silica gel (DCM/MeOH/NH₃ 98:2:0.2). The residue is stirred with DIPE. Yield: 17.2 mg (28% of theoretical); C₃₀H₃₁ClN₂ (M=455.033); calc.: molpeak (M+H)⁺: 455/457 (Cl); found: molpeak (M+H)⁺: 455/457 (Cl); R_{f} value: 0.82 (silica gel, 366 nm, DCM/MeOH/NH₃ 9:1:0.1).

### EXAMPLES 16.3 AND 16.4

### EXAMPLE 16.3

### ((E)-(S)-3-{4-[5-(4-chlorophenyl)pyridin-2-ylethynyl]phenyl}-1-methylbut-2-enyl)cyclopropylmethylamine

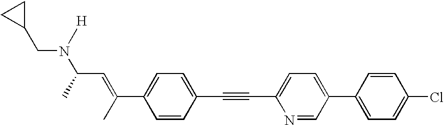

### EXAMPLE 16.4

### ((E)-(S)-3-{4-[5-(4-chlorophenyl)pyridin-2-ylethynyl]phenyl}-1-methylbut-2-enyl)-bis(cyclopropylmethyl)amine

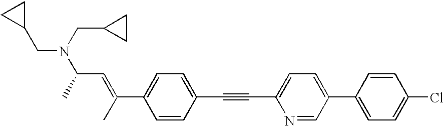

Examples 16.3 and 16.4: A solution of 180 mg (0.48 mmol) of (E)-(S)-3-{4-[5-(4-chlorophenyl)pyridin-2-ylethynyl]phenyl}-1-methylbut-2-enylamine and 0.04 mL (0.48 mmol) of cyclopropanecarboxaldehyde in 15 mL of THF is stirred for I hour at RT. Then 409 mg (1.93 mmol) of NaBH(OAc)₃ and 0.11 mL (1.93 mmol) of glacial acetic acid are added. The reaction mixture is stirred for 16 hours at RT. The solvent is eliminated in vacuo and the residue is diluted with 40 mL of EtOAc and 20 mL of semisaturated potassium carbonate solution. The organic phase is dried over sodium sulfate and the solvent is eliminated in vacuo. Further purification is carried out by column chromatography on silica gel (DCM/MeOH/N ₃ 9:1:0.1).

16.3: ((E)-(S)-3-{4-[5-(4-chlorophenyl)pyridin-2-ylethynyl]phenyl}-1-methylbut-2-enyl)cyclopropylmethylamine.

Yield: 100 mg (49% of theoretical); C₂₈H₂₇ClN₂ (M=426.980); calc.: molpeak (M+H)⁺: 427/429 (Cl); found: molpeak (M+H)⁺: 427/429 (Cl); R_{f} value: 0.39 (silica gel, 366 nm, DCM/MeOH/NH₃ 9:1:0.1).

16.4: ((E)-(S)-3-{4-[5-(4-chlorophenyl)pyridin-2-ylethynyl]phenyl}-1-methylbut-2-enyl)-bis(cyclopropylmethyl)amine.

Yield: 20.3 mg (9% of theoretical); C₃₂H₃₃CLN₂ (M=481.071); calc.: molpeak (M+H)⁺: 481/483 (Cl); found: molpeak (M+H)⁺: 481/483 (Cl); R_{f} value: 0.67 (silica gel, 366 nm, DCM/MeOH/NH₃ 9:1:0.1).

### EXAMPLE 16.5

### ((E)-(S)-3-{4-[5-(4-chlorophenyl)pyridin-2-ylethynyl]phenyl}-1-methylbut-2-enyl)cyclopropylmethylpropylamine

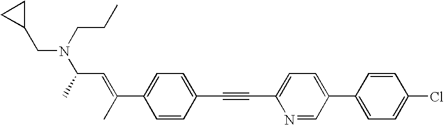

0.08 mL (0.84 mmol) of 1-bromopropane and 0.14 mL (0.84 mmol) of ethyldiisopropylamine are added to a solution of 45.0 mg (0.11 mmol) of ((E)-(S)-3-{4-[5-(4-chlorophenyl)pyridin-2-ylethynyl]phenyl}-1-methylbut-2-enyl)cyclopropylmethylamine in 1.0 mL of DMF and shaken for 50 hours at 60° C. The solvent is eliminated in vacuo. The residue is combined with 10 mL of semisaturated sodium bicarbonate solution and 20 mL of EtOAc. The organic phase is dried over sodium sulfate and the solvent is eliminated in vacuo. Further purification is carried out by column chromatography on silica gel (cyclohexane/EtOAc 1:2). Yield: 8.1 mg (16% of theoretical); C₃₁H₃₃ClN₂ (M=469.060); calc.: molpeak (M+H)⁺: 469/471 (Cl); found: molpeak (M+H)⁺: 469/471 (Cl); R_{f} value: 0.10 (silica gel, 366 nm, cyclohexane/EtOAc 1:2).

### EXAMPLE 17.1

### 1-{4-[5-(4-chlorophenyl)pyridin-2-ylethynyl]-2-methylphenoxymethyl}-cyclopropylamine

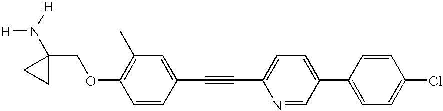 17.1a. (4-iodo-2-methylphenoxy)acetonitrile

7.447 g (53.9 mmol) of potassium carbonate is added to a solution of 13.0 g (53.9 mmol) of 4-iodo-2-methylphenol in 80.0 mL of DMF. Then 3.70 mL (53.9 mmol) of bromoacetonitrile dissolved in 20.0 mL of DMF is slowly added dropwise. The mixture is stirred for 24 hours at RT. The reaction mixture is filtered. The solvent is eliminated in vacuo. The residue is taken up in 400 mL of EtOAc and 200 mL of water. The organic phase is washed with 100 mL of saturated NaCl solution. The organic phase is dried over sodium sulfate and the solvent is eliminated in vacuo. Yield: 15.3 g (104% of theoretical); C₉H₈INO (M=273.070); calc.: molpeak (M+H)⁺: 273; found: molpeak (M+H)⁺: 273; R_{f} value: 0.62 (silica gel, cyclohexane/EtOAc 2:1).

17.1b. 1-(4-iodo-2-methylphenoxymethyl)cyclopropylamine

14.0 g (51.3 mmol) of (4-iodo-2-methylphenoxy)acetonitrile is dissolved in 210 mL of diethyl ether and cooled by means of a bath of ice/isopropanol. Then 17.1 mL (56.4 mmol) of titanium (IV) isopropoxide is carefully added dropwise. Then 34.2 mL (102.5 mmol, 3M in diethyl ether) of ethylmagnesium bromide solution is added dropwise and the mixture is stirred for 30 minutes. 13.0 mL (102.5 mmol) of boron trifluoride-diethyl ether complex is rapidly pipetted in at 10° C. After another 2 hours, 150 mL of 1M sodium hydroxide solution is added dropwise with further cooling. The reaction mixture is filtered. The phases of the filtrate are separated. The organic phase is washed with 150 mL saturated NaCl solution. The organic phase is dried over sodium sulfate and the solvent is eliminated in vacuo. Further purification is carried out using a gravity silica gel column (cyclohexane/EtOAc 4:1 after cyclohexane/EtOAc 2:1). Yield: 9.06 g (58.3% of theoretical); C₁₁H₁₄INO (M=303.139); calc.: molpeak (M+H)⁺: 304; found: molpeak (M+H)⁺: 304; R_{f} value: 0.07 (silica gel, cyclohexane/EtOAc 2:1).

17.1c. 1-{4-[5-(4-chlorophenyl)pyridin-2-ylethynyl]-2-methylphenoxymethyl }cyclopropyl-amine

Prepared according to General Method I from 1-(4-iodo-2-methylphenoxymethyl)cyclopropylamine (2.00 g, 6.60 mmol) and 5-(4-chlorophenyl)-2-ethynylpyridine (1.53 g, 6.60 mmol). Yield: 1.73 g (67% of theoretical); C₂₄H₂₁ClN₂O (M=388.889); calc.: molpeak (M+H)⁺: 389/391 (Cl); found: molpeak (M+H)⁺: 389/391 (Cl); R_{f} value: 0.59 (silica gel, DCM/MeOH/NH₃ 9:1:0.1).

### EXAMPLE 17.2 AND 17.3

### EXAMPLE 17.2

### (1-{4-[5-(4-chlorophenyl)pyridin-2-ylethynyl]-2-methylphenoxymethyl}-cyclopropyl)cyclopropylmethylamine

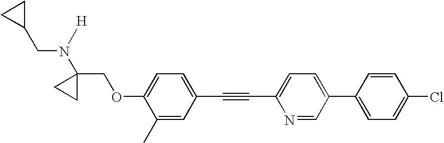

### EXAMPLE 17.3

### (1-{4-[5-(4-chlorophenyl)pyridin-2-ylethynyl]-2-methylphenoxymethyl}-cyclopropyl)-bis(cyclopropylmethyl)amine

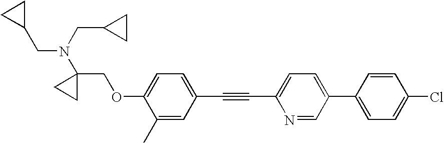

Examples 17.2 and 17.3: A solution of 1.00 g (2.57 mmol) of 1-{4-[5-(4-chlorophenyl)pyridin-2-ylethynyl]-2-methylphenoxymethyl}cyclopropylamine and 0.19 mL (2.57 mmol) of cyclopropanecarboxaldehyde in 75 mL of THF is stirred for 1 hour at RT. Then 2.18 g (10.3 mmol) of NaBH(OAc)₃ and 0.59 mL (10.3 mmol) of glacial acetic acid are added. The reaction mixture is stirred for 6 hours at RT. The solvent is eliminated in vacuo and the residue is diluted with 100 mL of EtOAc and 50 mL of semisaturated potassium carbonate solution. The organic phase is dried over sodium sulfate and the solvent is eliminated in vacuo. Further purification is carried out by column chromatography on silica gel (DCM/MeOH/NH₃ 98:2:0.2). The desired fractions are evaporated down and the respective residues are triturated with TBME.

17.2: ((E)-(R)-3-{4-[5-(4-chlorophenyl)pyridin-2-ylethynyl]phenyl}-1-methylbut-2-enyl)cyclopropylmethylamine.

Yield: 505 mg (44% of theoretical); C₂₈H₂₇ClN₂O (M=442.980); calc.: molpeak (M+H)⁺: 443/445 (Cl); found: molpeak (M+H)⁺: 443/445 (Cl); R_{f} value: 0.21 (silica gel, DCM/MeOHINH₃ 98:2:0.2).

17.3: (1-{4-[5-(4-chlorophenyl)pyridin-2-ylethynyl]-2-methylphenoxymethyl}cyclopropyl)-bis(cyclopropylmethyl)amine.

Yield: 57.7 mg (5% of theoretical); C₃₂H₃₃ClN₂O (M=497.070); calc.: molpeak (M+H)⁺: 497/499 (Cl); found: molpeak (M+H)⁺: 497/499 (Cl); retention time HPLC: 10.15 min (method A).

### EXAMPLE 17.4

### (1-{4-[5-(4-chlorophenyl)pyridin-2-ylethynyl]-2-methylphenoxymethyl}-cycloprolpyl)cyclopentylcyclopropylmethylamine

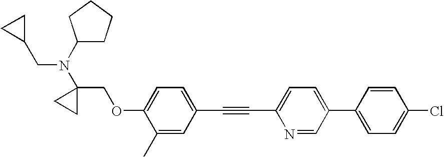

0.07 mL (0.72 mmol) of cyclopentanone is added at RT to a solution of 80.0 mg (0.18 mmol) of ((E)-(R)-3-{4-[5-(4-chlorophenyl)pyridin-2-ylethynyl]phenyl}-1-methylbut-2-enyl)cyclopropyl-methylamine in 8.0 mL of THF. After 15 minutes, 162 mg (0.72 mmol) of NaBH(OAc)₃ and 0.04 mL (0.72 mmol) of glacial acetic acid are added. After 3 hours (in which total dissolution is not achieved), 6 mL of DCM is added. After another 26 hours, 6 mL of MeOH, 0.07 mL (0.72 mmol) of cyclopentanone, 162 mg (0.72 mmol) of NaBH(OAc)₃, and 0.04 mL (0.72 mmol) of glacial acetic acid are added. After another 20 hours (dissolution, no reaction), 45.0 mg (0.72 mmol) of NaBH₃CN and 0.07 mL (0.72 mmol) of cyclopentanone are added. After stirring over the weekend, the solvent is eliminated in vacuo. The residue is stirred with DIPE. Further purification is carried out by column chromatography on silica gel (cyclohexane/EtOAc 4:1). Yield: 15.2 mg (16% of theoretical); C₂₅H₂₂ClNO (M=511.097); calc.: molpeak (M+H)⁺: 511/513; found: molpeak(M+H)⁺: 511/513; R_{f} value: 0.63 (silica gel, cyclohexane/EtOAc 2:1).

### EXAMPLE 17.5

### 5-(4-chlorophenyl)-2-{3-methyl-4-[1-(4-methylpiperidin-1-yl)cyclopropyl-methoxy]phenylethynyl}pyridine

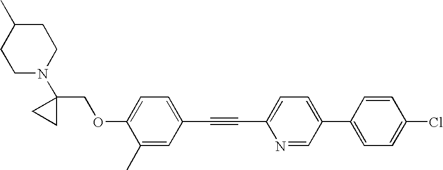

71.0 mg (0.51 mmol) of potassium carbonate and 47.8 mg (0.31 mmol) of 1,5-dibromo-3-methylpentane are added to a solution of 100 mg (0.26 mmol) of ((E)-(R)-3-{4-[5-(4-chlorophenyl)pyridin-2-ylethynyl]phenyl}-1-methylbut-2-enyl)cyclopropylmethylamine in 3.0 mL of DMF and shaken for 20 hours at 70° C. The reaction mixture is filtered and the solvent is eliminated in vacuo. The residue is diluted with 20 mL of EtOAc and 10 mL of semisaturated potassium carbonate solution. The organic phase is dried over sodium sulfate and the solvent is eliminated in vacuo. Further purification is carried out by preparative HPLC-MS. The desired fractions are evaporated dowvn and the residue triturated with PE. Yield: 3.2 mg (3% of theoretical); C₃₀H₃₁ClN₂O (M=471.033); calc.: molpeak (M+H)⁺: 471/473 (Cl); found: molpeak (M+H)⁺: 471/473 (Cl); retention time HPLC: 9.60 min (method A).

### EXAMPLE 17.6

### (1-{4-[5-(4-chlorophenyl)pyridin-2-ylethynyl]-2-methylphenoxymethyl}-cyclopropyl)cyclopropylmethylpropylamine

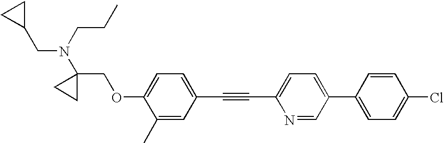

0.07 mL (0.72 mmol) of 1-bromopropane and 0.04 mL (0.72 mmol) of ethyldiisopropylamine are added to a solution of 80.0 mg (0.18 mmol) of ((E)-(R)-3-{4-[5-(4-chlorophenyl)pyridin-2-ylethynyl]phenyl}-1-methylbut-2-enyl)cyclopropylmethylamine in 1.7 mL of DMF and shaken for 4 hours at 70° C. The solvent is eliminated in vacuo. The residue is diluted with 30 mL of EtOAc and 10 mL of semisaturated potassium carbonate solution. The organic phase is dried over sodium sulfate and the solvent is eliminated in vacuo. Further purification is carried out by preparative HPLC-MS. Yield: 17.8 mg (20% of theoretical); C₃₁H₃₃ClN₂O (M=485.059); calc.: molpeak (M+H)⁺: 485/487 (Cl); found: molpeak (M+H)⁺: 485/487 (Cl); retention time HPLC: 6.60 min (method A).

### EXAMPLE 17.7

### (1-{4-[5-(4-chlorophenyl)pyridin-2-ylethynyl]-2-methylphenoxymethyl}-cyclopropyl)cyclopentylamine

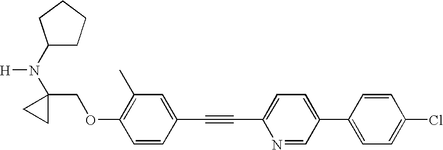

0.46 mL (5.17 mmol) of cyclopentanone is added to a solution of 500 mg (1.29 mmol) of 1-{4-[5-(4-chlorophenyl)pyridin-2-ylethynyl]-2-methylphenoxymethyl}cyclopropylamine in 50 mL of THF and the mixture is stirred for 15 minutes at RT. Then 1.15 g (5.17 mmol) of sodium triacetoxyborohydride and 0.30 mL (5.17 mmol) of AcOH are added. The reaction solution is stirred for 48 hours at RT, then neutralized with 30 mL of semisaturated sodium bicarbonate solution, and extracted with 40 mL of EtOAc, and the organic phase is dried over sodium sulfate. After the desiccant and solvent have been eliminated the residue is purified by chromatography (silica gel, cyc/EtOAc 2:1). The fractions containing the product are evaporated down in vacuo, the residue is triturated with isopropanol, suction filtered, and dried. Yield: 210 mg (58% of theoretical); C₂₉H₂₉ClN₂O (M=457.006); calc.: molpeak (M+H)⁺: 457/459 (Cl); found: molpeak (M+H)⁺: 457/459 (Cl); R_{f} value: 0.18 (silica gel, cyclohexane/EtOAc 2:1).

### EXAMPLE 18.1

### 5-(4-chlorophenyl)-2-[4-((S)-2-pyrrolidin-1-ylmethylpyrrolidin-1-yl)phenyl-ethlynyl]pyridine

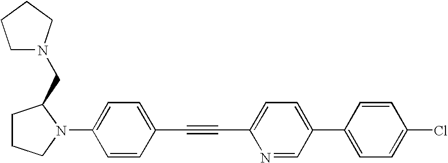

A suspension of 4.00 g (9.62 mmol) of 5-(4-chlorophenyl)-2-(4-iodophenylethynyl)pyridine (Example 8b), 3.00 g (19.4 mmol) of (S)-(+)-(2-pyrrolidinylmethyl)pyrrolidine, 187 mg (0.96 mmol) of CuI, and 0.74 g (3.85 mmol) of N,N-diethyl-2-hydroxybenzamide in 10 mL of DMF is evacuated several times and charged with argon. Then 4.72 g (19.2 mmol) of potassium phosphate monohydrate is added, the mixture is evacuated and charged with argon. The mixture is stirred overnight at 100° C. The reaction mixture is diluted with DCM and washed three times with 5% NH₃ solution and several times with water. The organic phase is dried over magnesium sulfate, filtered through activated charcoal, and the solvent is eliminated in vacuo. Further purification is carried out by column chromatography on silica gel (DCM/MeOH 9:1). Yield: 2.50 g (59.0% of theoretical); C₂₈H₂₈ClN₃ (M=441.995); calc.: molpeak (M+H)⁺: 442/444 (Cl); found: molpeak (M+H)⁺: 442/444 (Cl); R_{f} value: 0.35 (silica gel, DCM/MeOH 9:1).

### EXAMPLE 18.2

### 5-(4-chlorophenyl)-2-{4-[3-(4-methylpiperidin-1-yl)prop-1-ynyl]phenylethynyl}-pyridine

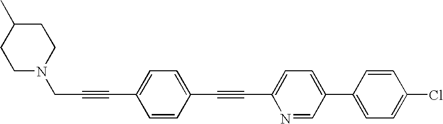 18.2a. 2-(4-bromophenylethynyl)-5-(4-chlorophenyl)pyridine

Prepared according to General Method I from 4-bromoiodobenzene (6.27 g, 21.5 mmol) and 5-(4-chlorophenyl)-2-ethynylpyridine (5.00 g, 21.5 mmol) in acetonitrile. Yield: 8.10 g (quant. yield); C₁₉H₁₁BrClN (M=368.654); calc.: molpeak (M+H)⁺: 368/370/372 (BrCl); found: molpeak (M+H)⁺: 368/370/372 (BrCl); R_{f} value: 0.73 (silica gel, DCM/PE 1:1).

18.2b. 2-(4-iodophenylethynyl)-5-(4-chlorophenyl)pyridine

Prepared according to General Method II from 2-(4-bromophenylethynyl)-5-(4-chlorophenyl)pyridine (8.10 g, 22.0 mmol). Yield: 6.80 g (74% of theoretical); C₁₉H₁₁ClIN (M=415.665); calc.: molpeak (M+H)⁺: 416/418 (Cl); found: molpeak (M+H)⁺: 416/418 (Cl); retention time HPLC: 7.9 min (method B).

18.2c. 3-{4-[5-(4-chlorophenyl)pyridin-2-ylethynyl]phenyl}prop-2-in-1-ol

A solution of 0.70 g (1.68 mmol) of 2-(4-iodophenylethynyl)-5-(4-chlorophenyl)pyridine, 98.3 μL (1.68 mmol) of prop-2-yn-1-ol, and 0.49 mL (3.54 mmol) of triethylamine in 10 mL acetonitrile is evacuated and charged with argon. 15.1 mg (0.02 mmol) of PdCl₂(dppf) and 3.53 mg (0.02 mmol) of CuI are added and the mixture is again evacuated and charged with argon. The reaction is stirred overnight at RT. The purification is carried out by column chromatography on silica gel (PE/EtOAc 1:1). Yield: 430 mg (74% of theoretical); C₂₂H₁₄ClNO (M=343.805); calc.: molpeak (M+H)⁺: 344/346 (Cl); found: molpeak (M+H)⁺: 344/346 (Cl); R_{f} value: 0.57 (silica gel, PE/EtOAc 1:1).

18.2d. 5-(4-chlorophenyl)-2-[4-(3-chloroprop-1-ynyl)phenylethynyl]pyridine

A solution of 39.6 μL of thionyl chloride in 2 mL of DCM is added at −10° C. to a solution of 150 mg (0.44 mmol) of 3-{4-[5-(4-chlorophenyl)pyridin-2-ylethynyl]phenyl}prop-2-yn-1-ol in 5 mL of DCM. The reaction solution is stirred for 30 minutes at 0° C. and overnight at RT. 5 mL of saturated sodium bicarbonate solution is added and the mixture is extracted with DCM. The organic phase is dried over magnesium sulfate and the solvent is eliminated in vacuo. Yield: 145 mg (92% of theoretical); C₂₂H₁₃Cl₂N (M=362.251); calc.: molpeak (M+H)⁺: 362/364/366 (2Cl); found: molpeak (M+H)⁺: 362/364/366 (2Cl); retention time HPLC: 7.49 min (method B).

18.2e. 5-(4-chlorophenyl)-2-{4-[3-(4-methylpiperidin-1-yl)prop-1-ynyl]phenylethynyl}pyridine 142 μL (1.20 mmol) of 4-methylpiperidine is added to a solution of 145 mg (0.40 mmol) of 5-(4-chlorophenyl)-2-[4-(3-chloroprop-1-ynyl)phenylethynyl]pyridine in 2 mL of DMF and stirred overnight at 70° C. After cooling, a solid is precipitated out, which is filtered off. Yield: 46.0 mg (27.0% of theoretical); C₂₈H₂₅ClN₂ (M=424.964); calc.: molpeak (M+H)⁺: 425/427 (Cl); found: molpeak (M+H)⁺: 425/427 (Cl); retention time HPLC: 6.01 min (method A).

### EXAMPLE 18.3

### (S)-1′-{4-[5-(4-chlorophenyl)pyridin-2-ylethynyl]phenyl}-4-methyl-[1,3′]-bipiperidinyl

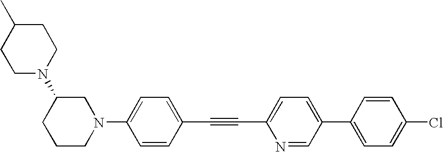 18.3a. (S)-1-(4-bromophenyl)piperidin-3-ylamine

1.00 g (5.00 mmol) of tert-butyl (S)-piperidin-3-yl-carbamate, 97.2 mg (0.50 mmol) of CuI, 2.12 g (10.0 mmol) of potassium phosphate, and 0.56 mL (10.0 mmol) of ethane-1,2-diol are added to a solution of 1.41 g (5.0 mmol) of 4-bromoiodobenzene in 10 mL of isopropanol, and the mixture is evacuated several times and charged with argon. The mixture is stirred overnight at 90° C. At RT, 100 mL of ⅓-concentrated HCl is added and the mixture is stirred for 1 hour. Then it is extracted twice with TBME. The aqueous phase is made alkaline with NH₃ solution and extracted several times with TBME. The organic phase is washed with a little water, dried over magnesium sulfate, filtered through activated charcoal, and the solvent is eliminated in vacuo. The residue is dissolved in 2 mL of DCM and purified by column chromatography (Hyperprep, gradient: 10% B towards 90% B in 12 minutes, then 5 minutes 90% B). Then the residue is taken up in a little water, neutralized with sodium bicarbonate and extracted with DCM. The organic phase is dried over magnesium sulfate and the solvent is eliminated in vacuo. Yield: 250 mg (20% of theoretical); C₁₁H₁₅BrN₂ (M=255.200); calc.: molpeak (M+H)⁺: 255/257 (Br); found: molpeak (M+H)⁺: 255/257 (Br); retention time HPLC: 6.46 min (method B).

18.3b. (S)-1′-(4-bromophenyl)-4-methyl-[1,3′]-bipiperidinyl

286 mg (1.18 mmol) of 1,5-dibromo-3-methylpentane and 325 mg (2.35 mmol) of potassium carbonate are added to a solution of 250 mg (0.98 mmol) of (S)-1-(4-bromophenyl)piperidin-3-ylamine in 10 mL of DMF and the mixture is stirred overnight at 50° C. The solvent is eliminated in vacuo. The residue is taken up in water, made alkaline with 2N sodium hydroxide solution, and extracted with TBME. The organic phase is dried over magnesium sulfate and the solvent is eliminated in vacuo. The purification is carried out 4by column chromatography (Hyperprep, gradient: 10% B towards 90% B in 12 minutes, then 5 minutes 90% B). The residue is again taken up in water, made alkaline with 2N sodium hydroxide solution, and extracted with TBME. The organic phase is dried over magnesium sulfate and the solvent is eliminated in vacuo. Yield: 125 mg (38% of theoretical); C₁₇H₂₅BrN₂ (M=337.298); calc.: molpeak (M+H)⁺: 337/339 (Br); found: molpeak (M+H)⁺: 337/339 (Br); R_{f} value: 0.40 (silica gel, DCM/MeOH/NH₃ 9:1:0.1).

18.3c. (S)-1′-(4-iodophenyl)-4-methyl-[1,3′]-bipiperidinyl

Prepared according to General Method II from (S)-1′-(4-bromophenyl)-4-methyl-[1,3′]-bipiperidinyl (125 mg, 0.37 mmol). Yield: 120 mg (84% of theoretical); C₁₇H₂₅IN₂ (M=384.298); calc.: molpeak (M+H)⁺: 385; found: molpeak (M+H)⁺: 385; retention time HPLC: 6.7 min (method A).

18.3d. (S)-1′-{4-[5-(4-chlorophenyl)pyridin-2-ylethynyl]phenyl}-4-methyl-[1,3′]-bipiperidinyl

Prepared according to General Method I from (S)-1′-(4-iodophenyl)-4-methyl-[1,3′]-bipiperidinyl (120 mg, 0.31 mmol) and 5-(4-chlorophenyl)-2-ethynylpyridine (66.8 mg, 0.31 mmol). Yield: 10.0 mg (7.0% of theoretical); C₃₀H₃₂ClN₃ (M=470.048); calc.: molpeak (M+H)⁺: 470/472 (Cl); found: molpeak (M+H)⁺: 470/472 (Cl); retention time HPLC: 6.0 min (method A).

### EXAMPLE 18.4

### (R)-1′-{4-[5-(4-chlorophenyl)pyridin-2-ylethynyl]phenyl}-4-methyl-[1,3′]-bipiperidinyl

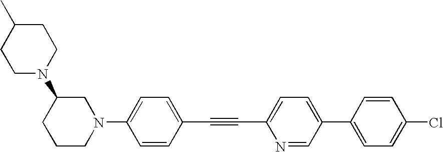 18.4a. tert-butyl [(R)-1-(4-bromophenyl)piperidin-3-yl]carbamate

2.00 g (10.0 mmol) of tert-butyl (R)-piperidin-3-ylcarbamate, 194 mg (1.00 mmol) of CuI, 4.25 g (20.0 mmol) of potassium phosphate, and 1.13 mL (20.0 mmol) of ethane-1,2-diol are added to a solution of 2.83 g (10.0 mmol) of 4-bromoiodobenzene in 20 mL of isopropanol, and the mixture is evacuated several times and charged with argon. It is stirred overnight at 90° C. At RT, 100 mL of ⅓-concentrated HCl is added and the mixture is stirred for 1 hour. Then it is extracted twice with TBME. The aqueous phase is made alkaline with NH₃ solution and extracted several times with TBME. The organic phase is washed with a little water, dried over magnesium sulfate, filtered through activated charcoal, and the solvent is eliminated in vacuo. Yield: 1.00 g (28% of theoretical); C₁₆H₂₃BrN₂O₂ (M=355.270); calc.: molpeak (M+H)⁺: 355/357 (Br); found: molpeak (M+H)⁺: 355/357 (Br).

18.4b. (R)-1-(4-bromophenyl)piperidin-3-ylamine

5.00 mL of trifluoroacetic acid is added to a solution of 1.00 g (2.82 mmol) of tert-butyl [(R)-1-(4-bromophenyl)piperidin-3-yl]carbamate in 50 mL of DCM and this is stirred overnight. The solvent is eliminated in vacuo. The residue is dissolved in DCM and made alkaline with 2N sodium hydroxide solution while being cooled. The organic phase is washed with water, dried over magnesium sulfate, and the solvent is eliminated in vacuo. Yield: 750 mg (quant. yield); C₁₁H₁₅BrN₂ (M=255.154); calc.: molpeak (M+H)⁺: 255/257 (Br); found: molpeak (M+H)⁺: 255/257 (Br); R_{f} value: 0.15 (silica gel, DCM/MeOH/NH₃ 9:1:0.1).

18.4c. (R)-1′-(4-bromophenyl)-4-methyl-[1,3′]-bipiperidinyl

832 mg (3.41 mmol) of 1,5-dibromo-3-methylpentane and 943 mg (6.82 mmol) of potassium carbonate are added to a solution of 725 mg (2.84 mmol) of (R)-1-(4-bromophenyl)piperidin-3-ylamine in 50 mL of DMF and the mixture is stirred overnight at 50° C. The reaction mixture is diluted with 50 mL of EtOAc and washed three times with semisaturated sodium bicarbonate solution. The organic phase is dried over magnesium sulfate and the solvent is eliminated in vacuo. Yield: 125 mg (13% of theoretical); C₁₇H₂₅BrN₂ (M=337.298); calc.: molpeak (M+H)⁺: 337/339 (Br); found: molpeak (M+H)⁺: 337/339 (Br).

18.4d. (R)-1′-(4-iodophenyl)-4-methyl-[1,3′]-bipiperidinyl

Prepared according to General Method II from (R)-1′-(4-bromophenyl)-4-methyl-[1,3′]-bipiperidinyl (125 mg, 0.37 mmol). Yield: 110 mg (77% of theoretical); C₁₇H₂₅IN₂ (M=384.298); calc.: molpeak (M+H)⁺: 385; found: molpeak (M+H)⁺: 385; retention time HPLC: 4.7 min (method B).

18.4e. (R)-1′-{4-[5-(4-chlorophenyl)pyridin-2-ylethynyl]phenyl}-4-methyl-[1,3′]-bipiperidinyl

Prepared according to General Method I from (R)-1′-(4-iodophenyl)-4-methyl-[1,3′]-bipiperidinyl (110 mg, 0.29 mmol) and 5-(4-chlorophenyl)-2-ethynylpyridine (66.8 mg, 0.31 mmol). Yield: 20.0 mg (15.0% of theoretical); C₃₀H₃₂ClN₃ (M=470.048); calc.: molpeak (M+H)⁺: 470/472 (Cl); found: molpeak (M+H)⁺: 470/472 (Cl); R_{f} value: 0.47 (silica gel, DCM/MeOH 9:1).

### EXAMPLE 19.1

### 5-(4-chlorophenyl)-2-{3-fluoro-4-[3-(4-methylpiperidin-1-yl)pyrrolidin-1-yl]phenylethynyl}pyridine

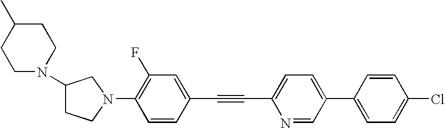 19.1a. 1-[1-(4-bromo-2-fluorophenyl)pyrrolidin-3-yl]-4-methylpiperidine

Under an argon atmosphere 17.9 mg (0.08 mmol) of palladium (II) acetate is added to a suspension of 800 mg (2.66 mmol) of 4-bromo-2-fluoro-1-iodobenzene, 447 mg (3.0 mmol) of 4-methyl-1-pyrrolidin-3-ylpiperidine (Amine A2), 1.21 g (40.0 mmol) of cesium carbonate, and 49.7 mg (0.08 mmol) of 2,2′-bis-(diphenylphosphino)-1,1′-binaphthalene in 15 mL of 1,4-dioxane and the reaction mixture is stirred overnight at 50° C. Another 49.7 mg (0.08 mmol) of 2,2′-bis(diphenylphosphino)-1,1′-binaphthalene and 17.9 mg (0.08 mmol) of palladium (II) acetate are added and the mixture is refluxed for 3 days. After cooling, it is combined with water and EtOAc, the phases are separated, and the organic phase is washed several times with water and dried over sodium sulfate. After the desiccant and solvent have been eliminated, the residue is purified by chromatography (silica gel, DCM/MeOH/NH₃ 95:5:0.5). Yield: 250 mg (28.0% of theoretical); C₁₆H₂₂BrFN₂ (M=341.262); calc.: molpeak (M+H)⁺: 341/343 (Br); found: molpeak (M+H)⁺: 341/343 (Br); retention time HPLC: 6.2 min (method A).

19.1b. 1-[1-(2-fluoro-4-iodophenyl)pyrrolidin-3-yl]-4-methylpiperidine

Prepared according to General Method II from 250 mg (0.73 mmol) of 1-[1-(4-bromo-2-fluorophenyl)pyrrolidin-3-yl]-4-methylpiperidine. Yield: 135 mg (47% of theoretical); C₁₆H₂₂FIN₂ (M=388.262); calc.: molpeak (M+H)⁺: 389; found: molpeak (M+H)⁺: 389; retention time HPLC: 6.0 min (method A).

19.1c. 5-(4-chlorophenyl)-2-{3-fluoro-4-[3-(4-methylpiperidin-1-yl)pyrrolidin-1-yl]phenylethynyl}pyridine

Prepared according to General Method I from 135 mg (0.35 mmol) of 1-[1-(2-fluoro-4-iodophenyl)pyrrolidin-3-yl]-4-methylpiperidine and 74 mg (0.35 mmol) of 5-(4-chlorophenyl)-2-ethynylpyridine (with Pd(dppf)Cl₂ as catalyst, triethylamine as base, and acetonitrile as solvent). Yield: 22 mg (13% of theoretical); C₂₉H₂₉ClFN₃ (M=474.012); calc.: molpeak (M+H)⁺: 474/476 (Cl); found: molpeak (M+H)⁺: 474/476 (Cl); retention time HPLC: 6.0 min (method A).

### EXAMPLE 20.1

### 5-(4-chlorophenyl)-2-{4-[(E)-1-methyl-3-(4-methylpiperidin-1-yl)propenyl]-phenylethynyl}pyridine

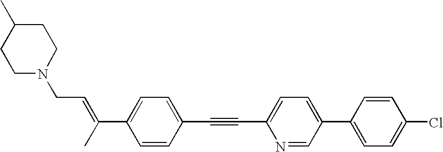 20.1a. (E)-3-(4-bromophenyl)but-2-enoic acid

Under an argon atmosphere 31.6 mg (0.14 mmol) of palladium (II) acetate and 171 mg (0.56 mmol) of tri-o-tolylphosphane are added to a solution of 4.1 g (14.1 mmol) of 1-bromo-4-iodo-benzene, 1.51 g (18.0 mmol) of (E)-but-2-enoic acid, and 3.91 mL (28.1 mmol) of triethylamine in 40 mL acetonitrile and the reaction mixture is refluxed overnight. After cooling, 20 mL of 1M HCl is added dropwise, the mixture is exhaustively extracted with EtOAc, and the combined organic phases are dried over magnesium sulfate. After the desiccant and solvent have been eliminated, the residue is purified by chromatography (silica gel, gradient PE/EtOAc 9:1 to 7:3). Yield: 700 mg (14% of theoretical); C₁₀H₉BrO₂ (M=241.081); calc.: molpeak (M−H)⁻: 239/241 (Br); found: molpeak (M−H)⁻: 239/241 (Br); retention time HPLC: 5.7 min (method E).

20.1b. (E)-3-(4-bromophenyl)but-2-en- 1-ol

659 mg (4.07 mmol) of CDI is added to a solution of 700 mg (2.03 mmol) of (E)-3-(4-bromophenyl)but-2-enoic acid in 10 mL of dry THF and the reaction mixture is stirred at 70° C. until the development of gas has ceased. A further 659 mg of CDI is added and the mixture is stirred for a further 2 hours at 60° C. After cooling, the reaction solution is added to a solution of 307 mg (8.13 mmol) of sodium borohydride in 10 mL of water (exothermic reaction) and stirred overnight at RT. The reaction mixture is acidified with dilute KHSO₄ solution, exhaustively extracted .vith EtOAc, and the combined organic phases are dried over magnesium sulfate. After the desiccant and solvent have been eliminated, the residue is purified by chromatography (silica gel, PE/EtOAc 7:3). Yield: 250 mg (54% of theoretical); C₁₀H₁₁BrO (M=227.098); calc.: molpeak (M)⁺: 226/228 (Br); found: molpeak (M)⁺: 226/228 (Br).

20.1c. 1-bromo-4-((E)-3-chloro-1-methylpropenyl)benzene

107 pL (1.32 mmol) of pyridine and one drop of DMF are added to a solution of 250 mg (1.10 mmol) of (E)-3-(4-bromophenyl)but-2-en-1-ol in 5 mL of DCM. After cooling in the ice bath, the solution is slowly combined with 96 μL (1.32 mmol) of thionyl chloride and the reaction mixture is stirred for 1 hour at this temperature and overnight at RT. It is combined with semisaturated sodium bicarbonate solution, extracted exhaustively with DCM, the combined organic phases are washed several times with water and dried over magnesium sulfate. After the desiccant and solvent have been eliminated, the residue is reacted further without purification. Yield: 240 mg (89% of theoretical); C₁₀H₁₀BrCl (M=245.543); calc.: molpeak (M)⁺: 244/246/248 (BrCl); found: molpeak (M)⁺: 244/246/248 (BrCl).

20.1d. 1-[(E)-3-(4-bromophenyl)but-2-enyl]-4-methylpiperidine

0.23 mL (1.96 mmol) of 4-methylpiperidine is added to a solution of 240 mg (0.98 mmol) of 1-bromo-4-((E)-3-chloro-1-methylpropenyl)benzene in 2 mL of DMF and the reaction solution is stirred for 2 hours at 45° C. and overnight at RT. It is evaporated down in vacuo, the residue is combined with water and extracted exhaustively with EtOAc, and the combined organic phases are washed twice with water and dried over magnesium sulfate. After the desiccant and solvent have been eliminated, the residue is reacted further without purification. Yield: 260 mg (86% of theoretical); C₁₆H₂₂BrN (M=308.257); calc.: molpeak (M+H)⁺: 308/310 (Br); found: molpeak (M+H)⁺: 308/310 (Br); retention time HPLC: 4.1 min (method B).

20.1e. 1-[(E)-3-(4-iodophenyl)but-2-enyl]-4-methylpiperidine

Prepared according to General Method II from 260 mg (0.84 mmol) of 1-[(E)-3-(4-bromophenyl)but-2-enyl]-4-methylpiperidine. Yield: 170 mg (57% of theoretical); C₁₆H₂₂IN (M=355.257); calc.: molpeak (M+H)⁺: 356; found: molpeak (M+H)⁺: 356; retention time HPLC: 4.3 min (method B).

20.1f. 5-(4-chlorophenyl)-2-{4-[(E)-1-methyl-3-(4-methylpiperidin-1-yl)propenyl]phenylethynyl}pyridine

Prepared according to General Method I from 160 mg (0.45 mmol) of 1-[(E)-3-(4-iodophenyl)but-2-enyl]-4-methylpiperidine and 106 mg (0.50 mmol) of 5-(4-chlorophenyl)-2-ethynylpyridine (with Pd(dppf)Cl₂ as catalyst, triethylamine as base, and THF as solvent). Yield: 30 mg (15% of theoretical); C₂₉H₂₉ClN₂ (M=441.007); calc.: molpeak (M+H)⁺: 441/443 (Cl); found: molpeak (M+H)⁺: 441/443 (Cl); retention time HPLC: 9.3 min (method A).

### EXAMPLE 20.2

### 1-((E)-3-{4-[5-(4-chlorophenyl)-3-fluoroperidin-2-ylethynyl]phenyl}but-2-enyl)-4-methylpiperidin-4-ol

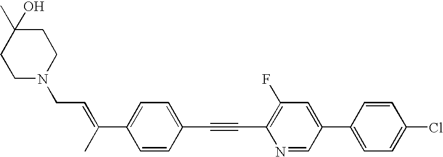 20.2a. 1-[(E)-3-(4-bromophenyl)but-2-enyl]-4-methylpiperidin-4-ol

0.24 mL (3.07 mmol) of thionyl chloride is added to a solution of 580 mg (2.55 mmol) of (E)-3-(4-bromophenyl)but-2-en-1-ol and 0.91 mL (5.11 mmol) of triethylamine in 10 mL of DCM cooled to −10° C. and the reaction mixture is brought to 0° C. within 30 minutes. Then it is combined with a solution of 882 mg (7.66 mmol) of 4-methylpiperidin-4-ol in 5 mL of DCM and the reaction solution is stirred for 2 hours at RT. 50 mL of semisaturated sodium bicarbonate solution is added, the mixture is extracted twice with 50 mL of DCM, and the combined organic phases are washed with water and dried over magnesium sulfate. After the desiccant and solvent have been eliminated, the residue is reacted further without purification. Yield: 180 mg (22% of theoretical); C₁₆H₂₂BrNO (M=324.256); calc.: molpeak (M+H)⁺: 324/326 (Br); found: molpeak (M+H)⁺: 324/326 (Br); retention time HPLC: 5.5 min (method A).

20.2b. 1-[(E)-3-(4-iodophenyl)but-2-enyl]-4-methylpiperidin-4-ol

Prepared according to General Method II from 180 mg (0.56 mmol) of 1-[(E)-3-(4-bromophenyl)but-2-enyl]-4-methylpiperidin-4-ol. Yield: 180 mg (87% of theoretical); C₁₆H₂₂INO (M=371.256); calc.: molpeak (M+H)⁺: 372; found: molpeak (M+H)⁺: 372; retention time HPLC: 5.7 min (method A).

20.2c. 1-((E)-3-{4-[5-(4-chlorophenyl)-3-fluoropyridin-2-ylethynyl]phenyl}but-2-enyl)-4-methylpiperidin-4-ol

Prepared according to General Method I from 80 mg (0.22 mmol) of I-[(E)-3-(4-iodophenyl)but-2-enyl]-4-methylpiperidin-4-ol and 50 mg (0.22 mmol) of 5-(4-chlorophenyl)-2-ethynyl-3-fluoropyridine (with Pd(dppf)Cl₂ as catalyst, piperidine as base, and acetonitrile as solvent). Yield: 37 mg (36% of theoretical); C₂₉H₂₈ClFN₂O (M=474.997); calc.: molpeak (M+H)⁺: 475/477 (Cl); found: molpeak (M+H)⁺: 475/477 (Cl); retention time HPLC: 5.0 min (method B).

### EXAMPLE 20.3

### 4-methyl-1-((E)-3-{4-[5-(4-methvlcyclohex-1-enyl)nyridin-2-ylethynyl]phenyl}-but-2-enyl)piperidin-4-ol

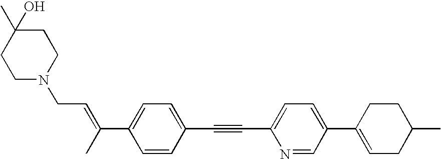

Prepared according to General Method I from 80 mg (0.22 mmol) of 1-[(E)-3-(4-iodophenyl)but-2-enyl]-4-methylpiperidin-4-ol and 50 mg (85%, 0.22 mmol) of 2-ethynyl-5-(4-methylcyclohex-1-enyl)pyridine (with Pd(dppf)Cl₂ as catalyst, piperidine as base, and acetonitrile as solvent). Yield: 13 mg (14% of theoretical); C₃₀H₃₆N₂O (M=440.620); calc.: molpeak (M+H)⁺: 441; found: molpeak (M+H)⁺: 441; R_{f} value: 0.10 (silica gel, DCM/MeOH 9:1).

### EXAMPLE 21.1

### (E)-5-(4-chlorophenyl)-2-{4-[3-(4,4-dimethylpiperidin-1-yl)propenyl]pyrid-3-ylethynyl}pyridine

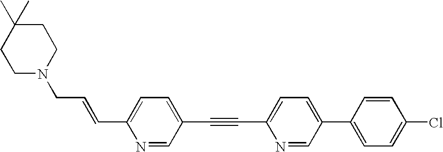

121 mg (1.06 mmol) of 4,4-dimethylpiperidine is added to a solution of 130 mg (0.36 mmol) of (E)-5-(4-chlorophenyl)-2-{4-[3-chloropropenyl]pyrid-3-ylethynyl}pyridine (Example 7.1e) in 3 mL of DMF and the reaction mixture is stirred for 2 hours at 60° C. After the reaction solution has been cooled, the product crystallizes out and is then filtered and dried. Yield: 18 mg (11% of theoretical); C₂₈H₂₈ClN₃ (M=441.995); calc.: molpeak (M+H)⁺: 442/444 (Cl); found: molpeak (M+H)⁺: 442/444 (Cl); retention time HPLC: 5.0 min (method A).

The following Examples are prepared analogously, in each case starting from 100 mg of (E)-5-(4-chlorophenyl)-2-{4-[3-chloropropenyl]pyrid-3-ylethynyl}pyridine, while the reaction solution is stirred overnight at 60° C. and after cooling is purified by HPLC without any further working up.

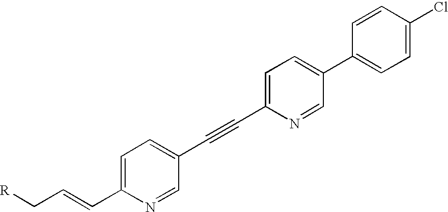

| | | | | | HPLC |
|---|---|---|---|---|---|
| | | | | Mass | retention time |
| Example | R | Yield (%) | Empirical formula | spectrum | (method) |
| 21.2 | 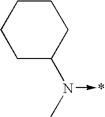 | 21 | C₂₈H₂₈ClN₃ | 442/444 [M + H]⁺ | 5.6 min (A) |
| 21.3 | 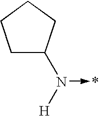 | 39 | C₂₆H₂₄ClN₃ | 414/416 [M + H]⁺ | 5.4 min (A) |
| 21.4 | 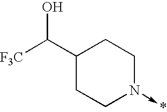 | 47 | C₂₈H₂₅ClF₃N₃O | 512/514 [M + H]⁺ | 5.5 min (A) |
| 21.5 | 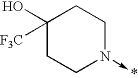 | 21 | C₂₇H₂₃ClF₃N₃O | 498/500 [M + H]⁺ | 7.7 min (A) |
| 21.6 | 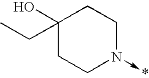 | 41 | C₂₈H₂₈ClN₃O | 458/460 [M + H]⁺ | 5.3 min (A) |
| 21.7 | 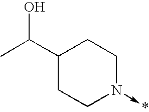 | 36 | C₂₈H₂₈ClN₃O | 458/460 [M + H]⁺ | 5.1 min (A) |
| 21.8 | 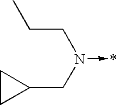 | 41 | C₂₈H₂₈ClN₃ | 442/444 [M + H]⁺ | 5.6 min (A) |
| 21.9 | 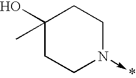 | 12 | C₂₇H₂₆ClN₃O | 444/446 [M + H]⁺ | 5.1 min (A) |

### EXAMPLE 22.1

### trans-5-(4-chlorophenyl)-2-{4-[(2-(4-methylpiperidin-1-ylmethyl)cyclopropyl]-phenylethynyl}pyridine

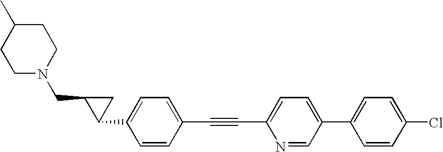 22.1a. ethyl trans-2-(4-bromophenyl)cyclopropanecarboxylate

5.7 g (55% in mineral oil, 129.5 mmol) of NaH is added batchwise to a solution of 34.4 g (153.0 mmol) of trimethylsulfoxonium iodide in 450 mL of anhydrous DMSO. After 1 hour, a solution of 30.0 g (117.6 mmol) of ethyl p-bromocinnamate in 400 mL of DMSO is slowly added dropwise, while the temperature rises to 30° C. The reaction mixture is stirred for 70 hours at RT, poured onto 1 L of saturated NaCl solution, extracted twice with 800 mL of EtOAc, and the organic phase is dried over sodium sulfate. After the desiccant and solvent have been eliminated, the residue is purified by chromatography (silica gel, Cyc). Yield: 17.7 g (56% of theoretical); C₁₂H₁₃BrO₂ (M=269.134); calc.: molpeak (M+H)⁺: 269/271 (Br); found: molpeak (M+H)⁺: 269/271 (Br); R_{f} value: 0.13 (silica gel, cyc); retention time (HPLC): 6.3 min (method B).

22.1b. trans-[2-(4-bromophenyl)cyclopropyl]methanol

At −10° C., a solution of 20.7 g (76.9 mmol) of ethyl trans-2-(4-bromophenyl)cyclopropanecarboxylate in 250 mL of THF is added dropwise to a solution of 100 mL (100 mmol, 1M in THF) of lithium aluminum hydride solution in 150 mL of THF and the reaction mixture is stirred for 1 hour at 0° C. The reaction solution is slowly combined with 20% KOH while cooling with an ice bath, stirred for 1 hour, and filtered to remove the insoluble matter. The phases of the filtrate are separated, and the organic phase is dried over sodium sulfate and evaporated down in vacuo. Yield: 16.7 g (95% of theoretical); C₁₀H₁₁BrO (M=227.098); calc.: molpeak (M−H)⁻: 225/227 (Br); found: molpeak (M−H)⁻: 225/227 (Br); R_{f} value: 0.47 (silica gel, EtOAc/cyc 1:1).

22.1c. trans-[2-(4-iodophenyl)cyclopropyl]methanol

Under a nitrogen atmosphere a solution of 12.0 g (52.8 mmol) of trans-[2-(4-bromophenyl)cyclopropyl]methanol and 2.0 g (10.6 mmol) of CuI in 52 mL of 1,4-dioxane is combined with 23.8 g (158.5 mmol) of NaI and 2.3 mL (21.1 mmol) of N,N′-dimethylethylenediamine. The reaction solution is shaken for 15 hours at 110° C., combined with 100 mL of semiconcentrated NH₃ solution and 300 mL of EtOAc, and the phases are separated and the organic phase is dried over sodium sulfate. After the desiccant and solvent have been eliminated, the residue is purified by chromatography (silica gel, gradient cyc/EtOAc 3:1 to 2:1). Yield: 10.0 g (69% of theoretical); C₁₀H₁₁IO (M=274.098); calc.: molpeak (M−H)⁻=273; found: molpeak (M−H)⁻=273; R_{f} value: 0.55 (silica gel, EtOAc/cyc 1:1).

22.1d. trans-(2-{4-[5-(4-chlorophenyl)pyridin-2-ylethynyl]phenyl}cyclopropyl)methanol

Under an argon atmosphere 0.3 g (0.37 mmol) of Pd(dppf)Cl₂ and 69.5 mg (0.37 mmol) of CuI are added to a solution of 5.0 g (18.2 mmol) of trans-[2-(4-iodophenyl)cyclopropyl]methanol, 4.7 g (21.9 mmol) of 5-(4-chlorophenyl)-2-ethynylpyridine, and 6.5 mL (36.5 mmol) of diisopropylamine in 180 mL of THF and the reaction mixture is stirred for 21 hours at RT. After the reaction has ended, the mixture is evaporated down to dryness in vacuo, and the residue is combined with 150 mL of EtOAc and 300 mL of water and filtered off. The crystals are combined with TBME, suction filtered again, and dried. Yield: 6.68 g (100% of theoretical); C₂₃H₁₈ClNO (M=359.848); calc.: molpeak (M+H)⁺=360/362 (Cl); found: molpeak (M+H)⁺=360/362 (Cl); R_{f} value: 0.38 (silica gel, EtOAc/cyc 1:1); retention time (HPLC): 6.2 min (method B).

22.1e. trans-2-[4-(2-chloromethylcyclopropyl)phenylethynyl]-5-(4-chlorophenyl)pyridine

860 μL (11.12 mmol) of thionyl chloride in 5 mL of DCM is added to a solution of 2.0 g (5.6 mmol) of trans-(2-{4-[5-(4-chlorophenyl)pyridin-2-ylethynyl]phenyl}cyclopropyl)methanol in 25 mL of DCM which has been cooled to 0° C. and the reaction solution is stirred for 30 minutes at 0° C. and 1.5 hours at RT. After the end of the reaction, the reaction mixture is combined at 0° C. with 40 mL of 5% sodium bicarbonate solution and 30 mL of DCM, the phases are separated, and the organic phase is dried over sodium sulfate. After the desiccant and solvent have been eliminated, the residue is purified by chromatography (silica gel, cyc/EtOAc 9:1). Yield: 520 mg (25% of theoretical); C₂₃H₁₇Cl₂N (M=378.293); calc.: molpeak (M+H)⁺=378/380/382 (2 Cl); found: molpeak (M+H)⁺=378/380/382 (2 Cl); R_{f} value: 0.43 (silica gel, EtOAc/cyc 1:1).

22.1f. trans-5-(4-chlorophenyl)-2-{4-[2-(4-methylpiperidin-1-ylmethyl)cyclopropyl]phenyl-ethynyl}pyridine

A solution of 80 mg (0.2 mmol) of trans-2-[4-(2-chloromethylcyclopropyl)phenylethynyl]-5-(4-chlorophenyl)pyridine and 100 μL (0.8 mmol) of 4-methylpiperidine is combined with 144 μL of ethyldiisopropylamine and stirred for 4 hours at 60° C. The reaction mixture is purified by HPLC, and the fractions containing the product are combined and lyophilized. Yield: 37 mg (40% of theoretical); C₂₉H₂₉ClN₂ (M=441.007); calc.: molpeak (M+H)⁺=441/443 (Cl); found: molpeak (M+H)⁺=441/443 (Cl); retention time (HPLC): 8.2 min (method A).

The following compounds are prepared analogously, in each case from 80 mg of trans-2-[4-(2-chloromethylcyclopropyl)phenylethynyl]-5-(4-chlorophenyl)pyridine and the corresponding amount of amine.

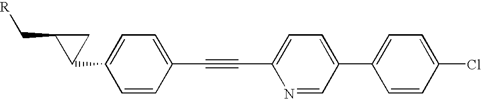

| | | | | | HPLC |
|---|---|---|---|---|---|
| | | | | Mass | retention time |
| Example | R | Yield (%) | Empirical formula | spectrum | (method) |
| 22.2 | 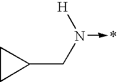 | 28 | C₂₇H₂₅ClN₂ | 413/415 [M + H]⁺ | 7.8 min (A) |
| 22.3 | 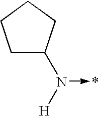 | 34 | C₂₈H₂₇ClN₂ | 427/429 [M + H]⁺ | 8.3 min (A) |
| 22.4 | 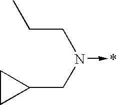 | 21 | C₃₀H₃₁ClN₂ | 455/457 [M + H]⁺ | 8.9 min (A) |
| 22.5 | 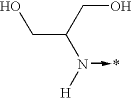 | 92 | C₂₆H₂₅ClN₂O₂ | 433/435 [M + H]⁺ | 7.2 min (A) |
| 22.6 | 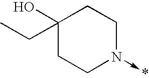 | 41 | C₃₀H₃₁ClN₂O | 471/473 [M + H]⁺ | 7.6 min (A) |
| 22.7 | 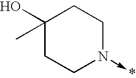 | 32 | C₂₉H₂₉ClN₂O | 457/459 [M + H]⁺ | 7.4 min (A) |

Some test methods for determining an MCH-receptor antagonistic activity will now be described. In addition, other test methods known to the skilled man may be used, e.g., by inhibiting the MCH-receptor-mediated inhibition of cAMP production, as described in M. Hoogduijn et al., Melanin-concentrating hormone and its receptor are expressed and functional in human skin, Biochem. Biophys. Res Commun. 296 (2002) pp. 698-701, and by biosensory measurement of the binding of MCH to the MCH receptor in the presence of antagonistic substances by plasmon resonance, as described in O. P. Karlsson and S. Lofas, Flow-Mediated On-Surface Reconstitution of G-Protein Coupled Receptors for Applications in Surface Plasmon Resonance Biosensors, Anal. Biochem. 300 (2002), pp. 132-138. Other methods of testing antagonistic activity to MCH receptors are contained in the references and patent documents mentioned hereinbefore, and the description of the test methods used is hereby incorporated in this application.

MCH-1 Receptor Binding Test

Method: MCH binding to hMCH-1R transfected cells
Species: Human
Test cell: hMCH-1R stably transfected into CHO/Galpha 16 cells
Results: IC₅₀ values

Membranes from CHO/Galpha 16 cells stably transfected with human hMCH-1R are resuspended using a syringe (needle 0.6×25 mm) and diluted in test buffer (50 mM HEPES, 10 mM MgCl₂, 2 mM EGTA, pH 7.00; 0.1% bovine serum albumin (protease-free), 0.021% bacitracin, 1 μg/mL aprotinin, 1 μg/mL leupeptin and 1 μM phosphoramidone) to a concentration of 5 to 15 μg/mL. 200 μL of this membrane fraction (contains 1 to 3 μg of protein) are incubated for 60 minutes at ambient temperature with 100 pM of ¹²⁵I-tyrosyl melanin concentrating hormone (¹²⁵I-MCH commercially obtainable from NEN) and increasing concentrations of the test compound in a final volume of 250 μL. After the incubation the reaction is filtered using a cell harvester through 0.5% PEI treated fiberglass filters (GF/B, Unifilter Packard). The membrane-bound radioactivity retained on the filter is then determined after the addition of scintillator substance (Packard Microscint 20) in a measuring device (TopCount of Packard). The non-specific binding is defined as bound radioactivity in the presence of 1 micromolar MCH during the incubation period. The analysis of the concentration binding curve is carried out on the assumption of one receptor binding site. Standard: Non-labeled MCH competes with labeled ¹²⁵I-MCH for the receptor binding with an IC50 value of between 0.06 and 0.15 nM. The KD value of the radioligand is 0.156 nM.

MCH-1 Receptor-Coupled Ca²⁺ Mobilization Test

Method: Calcium mobilization test with human MCH (FLIPR³⁸⁴)
Species: Human
Test cells: CHO/Galpha 16 cells stably transfected with hMCH-RI

Results: 1st measurement: % stimulation of the reference (MCH 10⁻⁶M); 2nd measurement: pKB value

| | | |
|---|---|---|
| Reagents: | HBSS (10x) | (GIBCO) |
| | HEPES buffer (1M) | (GIBCO) |
| | Pluronic F-127 | (Molecular Probes) |
| | Fluo-4 | (Molecular Probes) |
| | Probenecid | (Sigma) |
| | MCH | (Bachem) |
| | bovine serum albumin (protease-free) | (Serva) |
| | DMSO | (Serva) |
| | Ham's F12 | (BioWhittaker) |
| | FCS | (BioWhittaker) |
| | L-Glutamine | (GIBCO) |
| | Hygromycin B | (GIBCO) |
| | PENStrep | (BioWhittaker) |
| | Zeocin | (Invitrogen) |

Clonal CHO/Galpha 16 hMCH-R1 cells are cultivated in Ham's F12 cell culture medium (with L-glutamine; BioWhittaker; Cat. No.: BE12-615F). This contains per 500 mL: 10% FCS, 1% PENStrep, 5 mL of L-glutamine (200 mM stock solution), 3 mL of hygromycin B (50 mg/mL in PBS), and 1.25 mL of zeocin (100 μg/mL stock solution). One day before the experiment the cells are plated on a 384-well microtiter plate (black-walled with a transparent base, made by Costar) in a density of 2500 cells per cavity and cultivated in the above medium overnight at 37° C., 5% CO₂, and 95% relative humidity. On the day of the experiment, the cells are incubated with cell culture medium to which 2 mM Fluo-4 and 4.6 mM Probenicid have been added, at 37° C. for 45 minutes. After charging with fluorescent dye, the cells are washed four times with Hanks buffer solution (1×HBSS, 20 mM HEPES), which has been combined with 0.07% Probenicid. The test substances are diluted in Hanks buffer solution, combined with 2.5% DMSO. The background fluorescence of non-stimulated cells is measured in the presence of substance in the 384-well microtiter plate five minutes after the last washing step in the FLIPR³⁸⁴ apparatus (Molecular Devices; excitation wavelength: 488 nm; emission wavelength: bandpass 510 to 570 nm). To stimulate the cells MCH is diluted in Hanks buffer with 0.1% BSA, pipetted into the 384-well cell culture plate 35 minutes after the last washing step and the MCH-stimulated fluorescence is then measured in the FLIPR³⁸⁴ apparatus.

Data analysis:

1st measurement: The cellular Ca²⁺ mobilization is measured as the peak of the relative fluorescence minus the background and is expressed as the percentage of the maximum signal of the reference (MCH 10⁻⁶M). This measurement serves to identify any possible agonistic effect of a test substance.

2nd measurement: The cellular Ca²⁺ mobilization is measured as the peak of the relative fluorescence minus the background and is expressed as the percentage of the maximum signal of the reference (MCH 10⁻⁶M, signal is standardized to 100%). The EC50 values of the MCH dosage activity curve with and without test substance (defined concentration) are determined graphically by the GraphPad Prism 2.01 curve program. MCH antagonists cause the MCH stimulation curve to shift to the right in the graph plotted.

The inhibition is expressed as a pKB value:
pKB=log(EC_{50(testsubstance+MCH)}/EC_{50(MCH)}−1)−log c_{(testsubstance)}

The compounds according to the invention, including their salts, exhibit an MCH-receptor antagonistic activity in the tests mentioned above. Using the MCH-1 receptor binding test described above an antagonistic activity is obtained in a dosage range from about 10⁻¹⁰ to 10⁻⁵ M, particularly from 10⁻⁹ to 10⁻⁶ M.

The following IC₅₀ values were determined using the MCH-1 receptor binding test described above:

| Compound | | |
|---|---|---|
| according to | | |
| Example No. | Name of Substance | IC₅₀ value |
| 6.3 | (S)-1-(1-{5-[5-(4-chlorophenyl)pyridin-2- | 3.7 nM |
| | ylethynyl]pyridin-2-yl}pyrrolidin-3-yl) | |
| | piperidin-4-carboxylic acid amide | |
| 15.5 | ((E)-(R)-3-{4-[5-(4-chlorophenyl)pyridin-2- | 25 nM |
| | ylethynyl]phenyl}-1-methylbut-2- | |
| | enyl)cyclopropylmethylpropylamine | |

Some examples of formulations will be described hereinafter, wherein the term “active substance” denotes one or more compounds according to the invention, including their salts. In the case of one of the combinations with one or more active substances described, the term “active substance” also includes the additional active substances.

### EXAMPLE A

### Capsules for Powder Inhalation Containing 1 mg Active Substance

Composition:

1 capsule for powder inhalation contains:

| | |
|---|---|
| active substance | 1.0 mg |
| lactose | 20.0 mg |
| hard gelatin capsules | 50.0 mg |
| | 71.0 mg |

Preparation: The active substance is ground to the particle size required for inhalation. The ground active substance is homogeneously mixed with the lactose. The mixture is packed into hard gelatin capsules.

### EXAMPLE B

Inhalable Solution for Respimat® Containing 1 mg Active Substance

Composition:

1 spray contains:

| | | |
|---|---|---|
| active substance | 1.0 | mg |
| benzalkonium chloride | 0.002 | mg |
| disodium edetate | 0.0075 | mg |
| purified water | to 15.0 | μL |

Preparation: The active substance and benzalkonium chloride are dissolved in water and packed into Respimat® cartridges.

### EXAMPLE C

### Inhalable Solution for Nebulizer Containing 1 mg Active Substance

Composition:

1 vial contains:

| | | |
|---|---|---|
| active substance | 0.1 | g |
| sodium chloride | 0.18 | g |
| benzalkonium chloride | 0.002 | g |
| purified water | to 20.0 | mL |

Preparation: The active substance, sodium chloride, and benzalkonium chloride are dissolved in water.

### EXAMPLE D

### Propellant Type Metered Dose Aerosol Containing 1 mg Active Substance

Composition:

1 spray contains:

| | |
|---|---|
| active substance | 1.0 mg |
| lecithin | 0.1% |
| propellant gas | to 50.0 μL |

Preparation: The micronized active substance is homogeneously suspended in the mixture of lecithin and propellant gas. The suspension is transferred into a pressurised container with a metering valve.

### EXAMPLE E

### Nasal Spray Containing 1 mg Active Substance

Composition:

| | | |
|---|---|---|
| active substance | 1.0 | mg |
| sodium chloride | 0.9 | mg |
| benzalkonium chloride | 0.025 | mg |
| disodium edetate | 0.05 | mg |
| purified water | to 0.1 | mL |

Preparation: The active substance and the excipients are dissolved in water and transferred into a corresponding container.

### EXAMPLE F

### Injectable Solution Containing 5 mg of Active Substance per 5 mL

Composition:

| | | |
|---|---|---|
| active substance | 5 | mg |
| glucose | 250 | mg |
| human serum albumin | 10 | mg |
| glycofurol | 250 | mg |
| water for injections | to 5 | mL |

Preparation: Glycofurol and glucose are dissolved in water for injections (WfI); human serum albumin is added; active ingredient is dissolved with heating; made up to specified volume with WfI; transferred into ampoules under nitrogen gas.

### Example G

### Iniectable Solution Containing 100 mg of Active Substance per 20 mL

Composition:

| | | |
|---|---|---|
| active substance | 100 | mg |
| monopotassium dihydrogen phosphate = KH₂PO₄ | 12 | mg |
| disodium hydrogen phosphate = Na₂HPO₄.2H₂O | 2 | mg |
| sodium chloride | 180 | mg |
| human serum albumin | 50 | mg |
| Polysorbate 80 | 20 | mg |
| water for injections | to 20 | mL |

Preparation: Polysorbate 80, sodium chloride, monopotassium dihydrogen phosphate, and disodium hydrogen phosphate are dissolved in water for injections (WfI); human serum albumin is added; active ingredient is dissolved with heating; made up to specified volume with WfI; transferred into ampoules.

### EXAMPLE H

### Lyophilisate Containing 10 mg of Active Substance

Composition:

| | | |
|---|---|---|
| Active substance | 10 | mg |
| Mannitol | 300 | mg |
| human serum albumin | 20 | mg |

Preparation: Mannitol is dissolved in water for injections (WfI); human serum albumin is added; active ingredient is dissolved with heating; made up to specified volume with WfI; transferred into vials; freeze-dried.

Solvent for lyophilisate:

| | | |
|---|---|---|
| Polysorbate 80 = Tween 80 | 20 | mg |
| mannitol | 200 | mg |
| water for injections | to 10 | mL |

Preparation: Polysorbate 80 and mannitol are dissolved in water for injections (WfI); transferred into ampoules.

### EXAMPLE I

### Tablets Containing 20 mg of Active Substance

Composition:

| | | |
|---|---|---|
| active substance | 20 | mg |
| lactose | 120 | mg |
| maize starch | 40 | mg |
| magnesium stearate | 2 | mg |
| Povidone K 25 | 18 | mg |

Preparation: Active substance, lactose, and maize starch are homogeneously mixed; granulated with an aqueous solution of povidone; mixed with magnesium stearate; compressed in a tablet press; weight of tablet: 200 mg.

### EXAMPLE J

### Capsules Containing 20 mg Active Substance

Composition:

| | | |
|---|---|---|
| active substance | 20 | mg |
| maize starch | 80 | mg |
| highly dispersed silica | 5 | mg |
| magnesium stearate | 2.5 | mg |

Preparation: Active substance, maize starch, and silica are homogeneously mixed; mixed with magnesium stearate; the mixture is packed into size 3 hard gelatin capsules in a capsule filling machine.

### EXAMPLE K

### Suppositories Containing 50 mg of Active Substance

Composition:

| | | |
|---|---|---|
| active substance | 50 | mg |
| hard fat (Adeps solidus) | q.s. ad 1700 | mg |

Preparation: Hard fat is melted at about 38° C.; ground active substance is homogeneously dispersed in the molten hard fat; after cooling to about 35° C. it is poured into chilled moulds.

### EXAMPLE L

### Injectable Solution Containing 10 mg of Active Substance per 1 mL

Composition:

| | | |
|---|---|---|
| active substance | 10 | mg |
| mannitol | 50 | mg |
| human serum albumin | 10 | mg |
| water for injections | to 1 | mL |

Preparation: Mannitol is dissolved in water for injections (WfI); human serum albumin is added; active ingredient is dissolved with heating; made up to specified volume with WfI; transferred into ampoules under nitrogen gas.

1. A compound of formula (I) 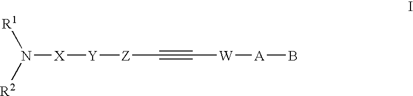
wherein:
R¹ and R² are each independently H, C₁₋₈-alkyl, C₃₋₇-cycloalkyl, or a phenyl or pyridinyl group optionally mono- or polysubstituted by identical or different groups R²⁰ and/or monosubstituted by nitro, wherein the alkyl or cycloalkyl group is optionally mono- or polysubstituted by identical or different groups R¹¹, and a —CH₂— group in position 3 or 4 of a 5-, 6-, or 7-membered cycloalkyl group is optionally may be replaced by —O—, —S—, or —NR¹³—, or
R¹ and R² form a C₃₋₈-alkylene bridge, wherein a —CH₂— group not adjacent to the N atom of the R¹R²N— group is optionally replaced by —CH═N—, —CH═CH—, —O—, —S—, —SO—, —(SO₂)—, —CO—, —C(═CH₂)—, or —NR¹³—, wherein in the alkylene bridge one or more H atoms are optionally replaced by identical or different groups R¹⁴, and the alkylene bridge is optionally substituted by one or two identical or different Cy groups such that the bond between the alkylene bridge and the group Cy is made via a single or double bond, via a common C atom forming a spirocyclic ring system, via two common adjacent C and/or N atoms forming a fused bicyclic ring system, or via three or more C and/or N atoms forming a bridged ring system;
X is a C_{1,6}-alkylene bridge independently substituted by one or more substituents selected from fluorine, chlorine, hydroxy, cyano, CF₃, C₁₋₄-alkyl, hydroxy-C₁₋₄-alkyl, C₃₋₆-cycloalkyl, and C₁₋₄-alkoxy, wherein two alkyl substituents thereof are optionally joined together to form a C₃₋₇-cycloalkyl group, or
a C₂₋₄-alkylenoxy or C₂₋₄-alkyleneimino bridge, wherein the imino group is optionally substituted by a C₁₋₄-alkyl group, and wherein the alkylene unit is independently substituted by one or more substituents selected from fluorine, chlorine, CF₃, hydroxy-C₁₋₄-alkyl, C₁₋₄-alkyl, and C₃₋₆-cycloalkyl, wherein two alkyl substituents thereof are optionally joined together to form a C₃₋₇-cycloalkyl group or a cyclo-C₄₋₆-alkyleneimino group, or
a C₃₋₆-alkenylene or C₃₋₆-alkynylene bridge optionally independently substituted by one or more substituents selected from fluorine, chlorine, CF₃, hydroxy-C₁₋₄-alkyl, C₁₋₄-alkyl, and C₃₋₆-cycloalkyl, wherein two alkyl substituents thereof are optionally joined together to form a C₃₋₇-cycloalkyl group or C₅₋₇-cycloalkenyl group;
W and Z are each independently a single bond or a C₁₋₂-alkylene bridge, while two adjacent C atoms are optionally joined together with an additional C₁₋₄-alkylene bridge, and one or two C atoms are optionally independently substituted by one or two identical or different C₁₋₃-alkyl groups, wherein two alkyl groups are optionally joined together to form a carbocyclic ring;
Y and A are each independently phenyl, pyridinyl, pyrimidinyl, pyrazinyl, pyridazinyl, naphthyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, chromanyl, chromen-4-onyl, thienyl, furanyl, benzothienyl, or benzofuranyl, each optionally mono- or polysubstituted at one or more C atoms by identical or different groups R²⁰, in the case of a phenyl ring, additionally optionally monosubstituted by nitro, and/or one or more NH groups are optionally substituted by R²¹;
B is independently Y, A, or C₁₋₆-alkyl, C₁₋₆-alkenyl, C₁₋₆-alkynyl, C₃₋₇-cycloalkyl, C₅₋₇-cycloalkenyl, C₃₋₇-cycloalkyl-C₁₋₃-alkyl, C₃₋₇-cycloalkenyl-C₁₋₃-alkyl, C₃₋₇-cycloalkyl-C₁₋₃-alkenyl, or C₃₋₇-cycloalkyl-C₁₋₃-alkynyl, wherein one or more C atoms are optionally independently mono- or polysubstituted by halogen and/or optionally monosubstituted by hydroxy or cyano and/or cyclic groups are optionally mono- or polysubstituted by identical or different groups R²⁰;
Cy is a saturated 3- to 7-membered carbocyclic group, an unsaturated 4- to 7-membered carbocyclic group, a phenyl group, a saturated 4- to 7-membered or unsaturated 5- to 7-membered heterocyclic group with an N, O, or S atom as heteroatom, a saturated or unsaturated 5- to 7-membered heterocyclic group with two or more N atoms or with one or two N atoms and an O or S atom as heteroatoms, or an aromatic heterocyclic 5- or 6-membered group with one or more identical or different heteroatoms selected from N, O, and/or S, wherein the saturated 6- or 7-membered groups thereof are optionally bridged ring systems with an imino, (C₁₋₄-alkyl)-imino, methylene, (C₁₋₄-alkyl)-methylene, or di-(C₁₋₄-alkyl)-methylene bridge, and the cyclic groups thereof are optionally mono- or polysubstituted at one or more C atoms by identical or different groups R²⁰, in the case of a phenyl group optionally additionally monosubstituted by nitro, and/or one or more NH groups are optionally substituted by R²¹;
R¹¹ is halogen, C₁₋₆-alkyl, C₂₋₆-alkenyl, C₂₋₆-alkynyl, R¹⁵—O—, R¹⁵—O—CO—, R¹⁵—CO—O—, cyano, R¹⁶R¹⁷N—, R¹⁸R¹⁹N—CO—, or Cy, wherein one or more C atoms thereof are optionally independently substituted by halogen, OH, CN, CF₃, C₁₋₃-alkyl, or hydroxy-C₁₋₃-alkyl;
R¹³ is independently R¹⁷;
R¹⁴ is halogen, cyano, C₁₋₆-alkyl, C₂₋₆-alkenyl, C₂₋₆-alkynyl, R¹⁵—O—, R¹⁵—O—CO—, R¹⁵—CO—, R¹⁵—CO—O—, R¹⁶—R¹⁷N—, R¹⁸R¹⁹N—CO—, R¹⁵—O—C₁₋₃-alkyl, R¹⁵—O—CO—C₁₋₃-alkyl, R¹⁵—SO₂—NH—, R¹⁵—O—CO—NH—C₁₋₃-alkyl, R¹⁵—SO₂—NH—C₁₋₃-alkyl, R¹⁵—CO—C₁₋₃-alkyl, R¹⁵—CO—O—C₁₋₃-alkyl, R¹⁶R¹⁷N—C₁₋₃-alkyl, R¹⁸R¹⁹N—CO—C₁₋₃-alkyl, or Cy—C₁₋₃-alkyl;
R¹⁵ is H, C₁₋₄-alkyl, C₃₋₇-cycloalkyl, C₃₋₇-cycloalkyl-C₁₋₃-alkyl, phenyl, phenyl-C₁₋₃-alkyl, pyridinyl, or pyridinyl-C₁₋₃-alkyl;
R¹⁶ is H, C₁₋₆-alkyl, C₃₋₇-cycloalkyl, C₃₋₇-cycloalkyl-C₁₋₃-alkyl, C₄₋₇-cycloalkenyl, C₄₋₇-cycloalkenyl-C₁₋₃-alkyl, ω-hydroxy-C₂₋₃-alkyl, ω-(C₁₋₄-alkoxy)-C₂₋₃-alkyl, amino-C₂₋₆-alkyl, C₁₋₄-alkyl-amino-C₂₋₆-alkyl, di-(C₁₋₄-alkyl)-amino-C₂₋₆-alkyl, or cyclo-C₃₋₆-alkyleneimino-C₂₋₆-alkyl;
R¹⁷ is independently R¹⁶ or phenyl, phenyl-C₁₋₃-alkyl, pyridinyl, C₁₋₄-alkylcarbonyl, hydroxycarbonyl-C₁₋₃-alkyl, C₁₋₄-alkoxycarbonyl, C₁₋₄-alkoxycarbonyl-C₁₋₃-alkyl, C₁₋₄-alkylcarbonylamino-C₂₋₃-alkyl, N-(C₁₋₄-alkylcarbonyl)-N-(C₁₋₄-alkyl)-amino-C₂₋₃-alkyl, C₁₋₄-alkylsulfonyl, C₁₋₄-alkylsulfonylamino-C₂₋₃-alkyl, or N-(C₁₋₄-alkylsulfonyl)-N(—C₁₋₄-alkyl)-amino-C₂₋₃-alkyl;
R¹⁸ and R¹⁹ are each independently H or C₁₋₆alkyl;
R²⁰ is independently R²² or halogen, hydroxy, cyano, C₁₋₆-alkyl, C₂₋₆-alkenyl, C₂₋₆-alkynyl, C₃₋₇-cycloalkyl, C₃₋₇-cycloalkyl-C₁₋₃-alkyl, hydroxy-C₁₋₃-alkyl, or R²²—C₁₋₃-alkyl;
R²¹ is C₁₋₄-alkyl, ω-hydroxy-C₂₋₆-alkyl, ω-C₁₋₄-alkoxy-C₂₋₆-alkyl, ω-C₁₋₄-alkyl-amino-C₂₋₆-alkyl, ω-di-(C₁₋₄-alkyl)-amino-C₂₋₆-alkyl, ω-cyclo-C₃₋₆-alkyleneimino-C₂₋₆-alkyl, phenyl, phenyl-C₁₋₃-alkyl, C₁₋₄-alkyl-carbonyl, C₁₋₄-alkoxy-carbonyl, C₁₋₄-alkylsulfonyl, aminosulfonyl, C₁₋₄-alkylaminosulfonyl, di-C₁₋₄-alkylaminosulfonyl, or cyclo-C₃₋₆-alkylene-iminosulfonyl;
R²² is pyridinyl, phenyl, phenyl-C_{1,3}-alkoxy, cyclo-C₃₋₆-alkyleneimino-C₂₋₄-alkoxy, OHC—, HO—N═HC—, C₁₋₄-alkoxy-N═HC—, C₁₋₄-alkoxy, C₁₋₄-alkylthio, carboxy, C₁₋₄-alkylcarbonyl, C₁₋₄-alkoxycarbonyl, aminocarbonyl, C₁₋₄-alkylaminocarbonyl, di-(C₁₋₄-alkyl)-aminocarbonyl, cyclo-C₃₋₆-alkyl-amino-carbonyl, cyclo-C₃₋₆-alkyleneimino-carbonyl, phenylaminocarbonyl, cyclo-C₃₋₆-alkyleneimino-C₂₋₄-alkyl-aminocarbonyl, C₁₋₄-alkyl-sulfonyl, C₁₋₄-alkyl-sulfinyl, C₁₋₄-alkyl-sulfonylamino, amino, C₁₋₄-alkylamino, di-(C₁₋₄-alkyl)-amino, C₁₋₄-alkyl-carbonylamino, cyclo-C₃₋₆-alkyleneimino, phenyl-C₁₋₃-alkylamino, N-(C₁₋₄-alkyl)phenyl-C₁₋₃-alkylamino, acetylamino, propionylamino, phenylcarbonyl, phenylcarbonylamino, phenylcarbonylmethylamino, hydroxy-C₂₋₃-alkylaminocarbonyl, (4-morpholinyl)carbonyl, (1-pyrrolidinyl)carbonyl, (1-piperidinyl)carbonyl, (hexahydro-1-azepinyl)carbonyl, (4-methyl-1-piperazinyl)carbonyl, methylenedioxy, aminocarbonylamino, or C₁₋₄-alkylaminocarbonylamino,
wherein in each of the abovementioned groups and residues one or more C atoms are additionally optionally mono- or polysubstituted by F and/or one or two C atoms are independently optionally monosubstituted by Cl or Br and/or one or more phenyl rings independently optionally contain one, two, or three substituents selected from F, Cl, Br, I, cyano, C₁₋₄-alkyl, C₁₋₄-alkoxy, difluoromethyl, trifluoromethyl, hydroxy, amino, C₁₋₃-alkylamino, di-(C₁₋₃-alkyl)-amino, acetylami no, aminocarbonyl, difluoromethoxy, trifluoromethoxy, amino-C₁₋₃-alkyl, C₁₋₃-alkylamino-C₁₋₃-alkyl-, and di-(C₁₋₃-alkyl)-amino-C₁₋₃-alkyl and/or are optionally monosubstituted by nitro, and
the H atom of any carboxy group present or an H atom bound to an N atom are each optionally replaced by a group which can be cleaved in vivo, and
the tautomers, enantiomers, salts, and mixtures thereof,
and excluding the following compounds:
(1-{5-[5-(4-chlorophenyl)pyridin-2-ylethynyl]pyridin-2-yl}pyrrolidin-3-yl)dimethylamine,
5′-[5-(4-chlorophenyl)pyridin-2-ylethynyl]-3-pyrrolidin-1-yl-3,4,5,6-tetrahydro-2H-[1,2′]-bipyridinyl,
1′-{5-[5-(4-chlorophenyl)pyridin-2-ylethynyl]pyridin-2-yl}-[1,3′]-bipyrrolidinyl,
{5-[5-(4-chlorophenyl)pyridin-2-ylethynyl]pyridin-2-yl}-(2-pyrrolidin-1-ylpropyl)amine,
5-(4-chlorophenyl)-2-[4-(1-methyl-2-piperidin-1-ylethoxy)phenylethynyl]pyridine,
5-(4-chlorophenyl)-2-[4-(3-piperidin-1-ylpyrrolidin-1-yl)phenylethynyl]pyridine,
5-(4-chlorophenyl)-2-{4-[2-(4-methylpiperidin-1-yl)propoxy]phenylethynyl}pyridine,
(1-{5-[5-(4-chlorophenyl)pyridin-2-ylethynyl]pyridin-2-yl}pyrrolidin-3-yl)-4-methylpiperidine,
5-(4-chlorophenyl)-2-[4-(2-methyl-2-piperidin-1-ylpropoxy)phenylethynyl]pyridine,
5-(4-chlorophenyl)-2-{4-[3-(4-methylpiperidin-1-yl)cyclohexyl]phenylethynyl}pyridine,
5-(4-chlorophenyl)-2-{4-[3-(4-methylpiperidin-1-yl)cyclohex-1-enyl]phenylethynyl}pyridine,
5-(4-chlorophenyl)-2-{4-[3-(4-methylpiperidin-1-yl)cyclopent-1-enyl]phenylethynyl}pyridine,
5-(4-chlorophenyl)-2-{4-[3-(4-methylpiperidin-1-yl)cyclopentyl]phenylethynyl}pyridine,
5-(4-chlorophenyl)-2-[4-(3-pyrrolidin-1-ylpropenyl)phenylethynyl]pyridine,
5-(4-chlorophenyl)-2-[4-(3-pyrrolidin-1-ylprop-1-ynyl)phenylethynyl]pyridine.

2. The compound of formula (I) according to claim 1, wherein:
R¹ and R² are each independently H, C₁₋₆-alkyl, C₃₋₅-alkenyl, C₃₋₅-alkynyl, C₃₋₇-cycloalkyl, hydroxy-C₃₋₇-cycloalkyl, C₃₋₇-cycloalkyl-C₁₋₃-alkyl, (hydroxy-C₃₋₇-cycloalkyl)-C₁₋₃-alkyl, hydroxy-C₂₋₄-alkyl, ω-NC-C₂₋₃-alkyl, C₁₋₄-alkoxy-C₂₋₄-alkyl, hydroxy-C₁₋₄-alkoxy-C₂₋₄-alkyl, C₁₋₄-alkoxy-carbonyl-C₁₋₄-alkyl, carboxyl-C₁₋₄-alkyl, amino-C₂₋₄-alkyl, C₁₋₄-alkyl-amino-C₂₋₄-alkyl, di-(C₁₋₄-alkyl)-amino-C₂₋₄-alkyl, cyclo-C₃₋₆-alkyleneimino-C₂₋₄-alkyl, pyrrolidin-3-yl, N-(C₁₋₄-alkyl)pyrrolidin-3-yl, pyrrolidinyl-C₁₋₃-alkyl, N-(C₁₋₄-alkyl)pyrrolidinyl-C₁₋₃-alkyl, piperidin-3-yl, piperidin-4-yl, N—(C₁₋₄-alkyl)piperidin-3-yl, N—(C₁₋₄-alkyl)piperidin-4-yl, piperidinyl-C₁₋₃-alkyl, N—(C₁₋₄-alkyl)piperidinyl-C₁₋₃-alkyl, tetrahydropyran-3-yl, tetrahydropyran-4-yl, phenyl, phenyl-C, ₁₋₃-alkyl, pyridyl, or pyridyl-C₁₋₃-alkyl, wherein one or more C atoms thereof are optionally independently mono- or polysubstituted by F, C₁₋₃-alkyl or hydroxy-C₁₋₃-alkyl, and/or one or two C atoms are optionally independently monosubstituted by Cl, Br, OH, CF₃, or CN, and the phenyl or pyridyl group are optionally mono- or polysubstituted by identical or different groups R²⁰, and, in the case of a phenyl group, is additionally optionally monosubstituted by nitro.

3. The compound of formula (I) according to claim 1, wherein:
R¹ and R² together with the N atom to which they are bound form a heterocyclic group which is selected from the meanings pyrrolidine, piperidine, 8-azabicyclo[3.2.1]octane, piperazine, wherein the free imine function is substituted by R¹³, and morpholine, wherein one or more H atoms may be replaced by identical or different groups R¹⁴, and/or the abovementioned heterocyclic groups may be substituted by one or two identical or different carbo- or heterocyclic groups Cy in such a way that the bond between the alkylene bridge and the group Cy is made via a single or double bond, via a common C atom forming a spirocyclic ring system, via two common adjacent C- and/or N atoms forming a fused bicyclic ring system, or via three or more C- and/or N atoms forming a bridged ring system.

4. The compound of formula (I) according to claim 1, wherein:
X is a propylene bridge independently substituted by one or more substituents selected from fluorine, chlorine, hydroxy, C₁₋₃-alkyl, and cyclopropyl, wherein two alkyl substituents thereof are optionally joined together to form a C₃₋₆-cycloalkyl group, or
an ethoxy or an ethyleneimino bridge, wherein the imino group thereof is optionally substituted by C₁₋₄-alkyl, independently substituted by one or more substituents selected from fluorine, chlorine, C₁₋₃-alkyl, and cyclopropyl, wherein two alkyl substituents thereof are optionally joined together to form a C₃₋₆-cycloalkyl group, or if an alkyl group is linked to an imino group, they are optionally joined together to form a pyrrolidine or piperidine group, or
a —CH₂—CH═CH— or —CH₂—C≡C— bridge which is optionally independently substituted by one or more substituents selected from fluorine, chlorine, C₁₋₃-alkyl, and cyclopropyl, wherein two alkyl substituents thereof are optionally joined together to form a C₃₋₆-cycloalkyl group.

5. The compound of formula (I) according to claim 1, wherein:
Z is a single bond or ethylene; and
W is a single bond.

6. The compound of formula (I) according to claim 1, wherein:
Y is phenyl, pyridinyl, pyrimidinyl, pyrazinyl, pyridazinyl, tetrahydronaphthyl, indolyl, dihydroindolyl, quinolinyl, dihydroquinolinyl, tetrahydroquinolinyl, isoquinolinyl, dihydroisoquinolinyl, tetrahydroisoquinolinyl, benzimidazolyl, benzoxazolyl, chromanyl, chromen-4-onyl, benzothienyl, or benzofuranyl, wherein the cyclic groups thereof are optionally mono- or polysubstituted at one or more C atoms by identical or different R²⁰ groups, and, in the case of a phenyl ring, is optionally additionally monosubstituted by nitro, and/or is optionally substituted at one or more N atoms by R²¹.

7. The compound of formula (I) according to claim 1, wherein:
A is phenyl, pyridinyl, pyrimidinyl, pyrazinyl, or pyridazinyl, each optionally mono- or polysubstituted at one or more C atoms by identical or different R²⁰ groups, and, in the case of a phenyl ring, is optionally additionally monosubstituted by nitro.

8. The compound of formula (I) according to claim 1, wherein:
B is phenyl, cyclohexenyl, pyridyl, thienyl, or furanyl, wherein the cyclic groups thereof are optionally mono- or polysubstituted at one or more C atoms by identical or different R²⁰ groups, and, in the case of a phenyl group, is optionally additionally monosubstituted by nitro.

9. The compound of formula (I) according to claim 1, wherein: 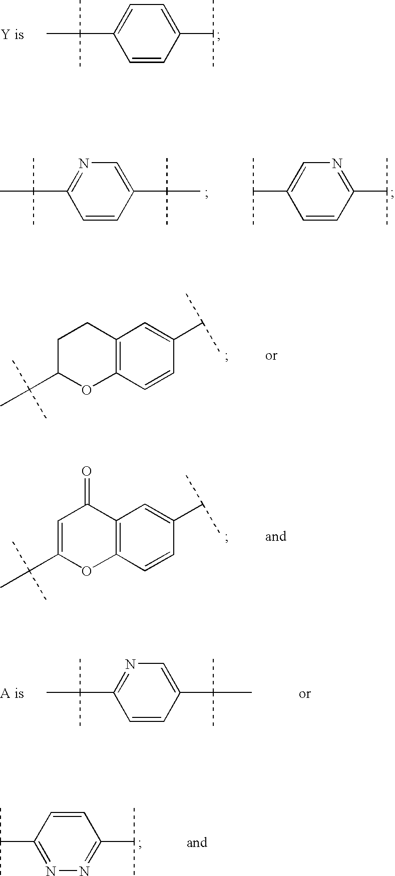
B is phenyl, cyclohexenyl, pyridyl, thienyl, or furanyl,
wherein Y and A are unsubstituted or monosubstituted by R²⁰, and B is optionally independently mono-, di-, or trisubstituted by R²⁰, and, in the case of a phenyl ring, is optionally additionally monosubstituted by nitro.

10. The compound of formula (I) according to claim 1, wherein:
R²⁰ is F, Cl, Br, I, OH, cyano, methyl, difluoromethyl, trifluoromethyl, ethyl, n-propyl, isopropyl, amino, acetyl, methoxy, difluoromethoxy, trifluoromethoxy, ethoxy, n-propoxy, or isopropoxy, wherein each R²⁰ is identical or different.

11. A physiologically acceptable salt of the compound according to claim 1.

12. A pharmaceutical formulation comprising the compound according to claim 1 and one or more physiologically acceptable excipients or inert carriers or diluents.

13. A pharmaceutical formulation comprising the compound according to claim 2 and one or more physiologically acceptable excipients or inert carriers or diluents.

14. A pharmaceutical formulation comprising the compound according to claim 3 and one or more physiologically acceptable excipients or inert carriers or diluents.

15. A pharmaceutical formulation comprising the physiologically acceptable salt according to claim 11 and one or more physiologically acceptable excipients or inert carriers or diluents.

16. The pharmaceutical formulation according to claim 12 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

17. The pharmaceutical formulation according to claim 13 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

18. The pharmaceutical formulation according to claim 14 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

19. The pharmaceutical formulation according to claim 15 further comprising a second active substance selected from the group consisting of active substances for the treatment of diabetes, active substances for the treatment of diabetic complications, active substances for the treatment of obesity, active substances for the treatment of high blood pressure, active substances for the treatment of hyperlipidemia or arteriosclerosis, active substances for the treatment of arthritis, active substances for the treatment of anxiety states, and active substances for the treatment of depression.

20. A method for influencing the eating behavior of a mammal comprising administering to the mammal an effective amount of the compound according to claim 1.

21. A method for influencing the eating behavior of a mammal comprising administering to the mammal an effective amount of the compound according to claim 2.

22. A method for influencing the eating behavior of a mammal comprising administering to the mammal an effective amount of the compound according to claim 3.

23. A method for influencing the eating behavior of a mammal comprising administering to the mammal an effective amount of the physiologically acceptable salt according to claim 11.

24. A method for reducing the body weight and/or for preventing an increase in the body weight of a mammal comprising administering to the mammal an effective amount of the compound according to claim 1.

25. A method for reducing the body weight and/or for preventing an increase in the body weight of a mammal comprising administering to the mammal an effective amount of the compound according to claim 2.

26. A method for reducing the body weight and/or for preventing an increase in the body weight of a mammal comprising administering to the mammal an effective amount of the compound according to claim 3.

27. A method for reducing the body weight and/or for preventing an increase in the body weight of a mammal comprising administering to the mammal an effective amount of the physiologically acceptable salt according to claim 11.

28. A method for preventing or treating a metabolic disorder or eating disorder in a mammal comprising administering to the mammal an effective amount of the compound according to claim 1.

29. A method for preventing or treating a metabolic disorder or eating disorder in a mammal comprising administering to the mammal an effective amount of the compound according to claim 2.

30. A method for preventing or treating a metabolic disorder or eating disorder in a mammal comprising administering to the mammal an effective amount of the compound according to claim 3.

31. A method for preventing or treating a metabolic disorder or eating disorder in a mammal comprising administering to the mammal an effective amount of the physiologically acceptable salt according to claim 11.

32. The method according to claim 28, wherein the metabolic disorder or eating disorder is obesity, bulimia, bulimia nervosa, cachexia, anorexia, anorexia nervosa, or hyperphagia.

33. The method according to claim 29, wherein the metabolic disorder or eating disorder is obesity, bulimia, bulimia nervosa, cachexia, anorexia, anorexia nervosa, or hyperphagia.

34. The method according to claim 30, wherein the metabolic disorder or eating disorder is obesity, bulimia, bulimia nervosa, cachexia, anorexia, anorexia nervosa, or hyperphagia.

35. The method according to claim 31, wherein the metabolic disorder or eating disorder is obesity, bulimia, bulimia nervosa, cachexia, anorexia, anorexia nervosa, or hyperphagia.

36. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 1.

37. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 2.

38. A method for preventing or treating diabetes, diabetic retinopathy, diabetic neuropathy, diabetic nephropathy, insulin resistance, pathological glucose tolerance, encephalorrhagia, cardiac insufficiency, arteriosclerosis, high blood pressure, arthritis, or gonitis in a mammal comprising administering to the mammal an effective amount of the compound according to claim 3.

39. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 1.

40. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 2.

41. A method for preventing or treating hyperlipidemia, cellulitis, fat accumulation, malignant mastocytosis, systemic mastocytosis, emotional disorders, affective disorders, depression, anxiety, sleep disorders, reproductive disorders, sexual disorders, memory disorders, epilepsy, forms of dementia, or hormonal disorders in a mammal comprising administering to the mammal an effective amount of the compound according to claim 3.

42. A method for preventing or treating urinary incontinence, hyperactive urinary bladder, urgency, nycturia, or enuresis in a mammal comprising administering to the mammal an effective amount of the compound according to claim 1.

43. A method for preventing or treating urinary incontinence, hyperactive urinary bladder, urgency, nycturia, or enuresis in a mammal comprising administering to the mammal an effective amount of the compound according to claim 2.

44. A method for preventing or treating urinary incontinence, hyperactive urinary bladder, urgency, nycturia, or enuresis in a mammal comprising administering to the mammal an effective amount of the compound according to claim 3.

45. A method for treating dependencies and/or withdrawal symptoms in a mammal comprising administering to the mammal an effective amount of the compound according to claim 1.

46. A method for treating dependencies and/or withdrawal symptoms in a mammal comprising administering to the mammal an effective amount of the compound according to claim 2.

47. A method for treating dependencies and/or withdrawal symptoms in a mammal comprising administering to the mammal an effective amount of the compound according to claim 3.
